(19) United States
(12) Patent Application Publication
(10) Pub. No.: US 2026/0159883 A1
(43) Pub. Date: Jun. 11, 2026

(54) METHODS AND COMPOSITIONS FOR ANALYSIS AND TREATMENT OF REPEAT EXPANSION DISORDERS

(71) Applicants: The Broad Institute, Inc., Cambridge, MA (US); PRESIDENT AND FELLOWS OF HARVARD COLLEGE, Cambridge, MA (US)

(72) Inventors: Steven McCarroll, Cambridge, MA (US); Robert E. Handsaker, Charlemont (MG); Nolan Kamitaki, Cambridge, MA (US); Nora Reed, Cambridge, MA (US); Tara McDonald, Boston, MA (US); Won-Seok Lee, Cambridge, MA (US)

(73) Assignees: The Broad Institute, Inc., Cambridge, MA (US); PRESIDENT AND FELLOWS OF HARVARD COLLEGE, Cambridge, MA (US)

(21) Appl. No.: 19/363,207

(22) Filed: Oct. 20, 2025

Related U.S. Application Data
(63) Continuation of application No. PCT/US24/25389, filed on Apr. 19, 2024.
(60) Provisional application No. 63/461,164, filed on Apr. 21, 2023.
(60) Provisional application No. 63/558,354, filed on Feb. 27, 2024.

(51) Int. Cl. C12Q 1/6869; C12N 15/10; C12Q 1/686; C12Q 1/6883

(52) U.S. Cl. CPC C12Q 1/6869; C12N 15/1096; C12Q 1/686; C12Q 1/6883

(57) ABSTRACT

Methods and compositions for analysis and treatment of repeat expansion disorders are described. Labeled amplicons of a variable repeat region of a gene may be generated, the generating using primers that introduce at least one molecular label to respective nucleic acid molecules of origin of a biological sample. The labeled amplicons may be sequenced to generate sequencing reads having the at least one molecular label incorporated. A sequence repeat length distribution of the variable repeat region in at least a portion of the biological sample may be generated based on the sequencing reads.

### RELATED APPLICATIONS

This application claims priority to claims the benefit of and priority to International Application No. PCT/US24/25389, filed on Apr. 19, 2024, which claims the benefit of and priority to U.S. Provisional Patent Application Ser. No. 63/461,164, filed on Apr. 21, 2023, and to U.S. Provisional Patent Application Ser. No. 63/558,354, filed on Feb. 27, 2024, the entire disclosures of which are hereby incorporated by reference in their entireties.

### REFERENCE TO SEQUENCE LISTING SUBMITTED ELECTRONICALLY

This application includes a Sequence Listing, which has been filed electronically in XML format and is hereby incorporated by reference into the specification. The XML file containing the Sequence Listing is named “11108_SL.xml.xml,” was created on Oct. 20, 2025, and is 30,530 bytes in size.

### BACKGROUND

Repeat expansion disorders (or diseases) are inherited genetic disorders characterized by the expansion of a repetitive sequence of nucleotides (e.g., a microsatellite) within a specific gene. For example, these repetitive sequences, which typically include three to six nucleotides repeated multiple times, are expanded during DNA replication, DNA maintenance, or DNA repair, leading to mosaicism, in which different cells have varying sequence repeat lengths. Expansion of the sequence repeat length beyond a certain threshold can lead to cellular toxicity and disease.

One example of a repeat expansion disorder is Huntington disease (also called Huntington's disease and abbreviated as HD), an autosomal dominant neurodegenerative disorder that causes progressive movement, cognitive, and psychological symptoms through the degeneration of specific types of neurons. HD involves inheritance of a CAG sequence repeat of 36 or more CAGs in exon 1 of the huntingtin (HTT) gene (CAGₙ, encoding polyglutamine). A length of the polyglutamine stretch in a resulting protein corresponds a length n of the CAG, repeat. The length of the inherited (e.g., germline) CAG sequence repeat is inversely correlated age of onset of disease symptoms. Generally, the greater the number of CAG repeat units, the earlier the onset. Moreover, the CAG sequence repeat is somatically unstable, leading to length variation (mosaicism) in brain tissue. By way of example, in individuals with HD, the somatic instability of the CAG sequence repeat length may cause the number of CAG repeat units to increase, leading to disease onset or progression. A similar set of relationships may be present in other repeat expansion disorders, such as myotonic dystrophy and several ataxias. Because the length of the DNA sequence repeat can change within affected tissues over time and is variable across the individual cells within a tissue, measurements of the length of the DNA sequence repeat may provide insight into disease processes and prognoses and enable potential therapeutic interventions to be evaluated.

### SUMMARY

Methods and compositions for analysis and treatment of repeat expansion disorders are described. Labeled amplicons of a variable repeat region of a gene may be generated, said generating using primers that introduce at least one molecular label to respective nucleic acid molecules of origin of a biological sample. The labeled amplicons may be sequenced to generate sequencing reads having the at least one molecular label incorporated. A sequence repeat length distribution of the variable repeat region in at least a portion of the biological sample may be generated based on the sequencing reads.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ methods and compositions for analysis and treatment of repeat expansion disorders.

FIGS. 2A and 2B depict an example workflow in an implementation of preparing single-cell target-sequence sequencing data for repeat length distribution analysis.

FIG. 3 depicts an illustrative example process for synthesizing cell barcoded and UMI-labeled cDNA from RNA for single cell/nucleus sequencing for sequence repeat length distribution analysis.

FIG. 4 depicts an example workflow in an implementation of preparing a genomic DNA sample for sequence repeat length distribution analysis.

FIG. 5 depicts an illustrative example amplification reaction for introducing unique molecular identifiers (UMIs) for labeling individual DNA molecules in a bulk sample.

FIG. 6 depicts a simplified example of sequence repeat length distributions in read families.

FIG. 7 depicts a simplified example of sequence repeat length distributions in a biological sample.

FIG. 8 depicts an example procedure in which methods and compositions for analysis and treatment of repeat expansion disorders is performed.

FIG. 9 depicts an example procedure in which a single cell/nucleus RNA sequencing sample is prepared for sequence length distribution analysis.

FIG. 10 depicts an example procedure in which a genomic DNA sample is prepared for sequence length distribution analysis.

FIG. 11 illustrates an example system including various components of an example device that can be implemented as any type of computing device as described and/or utilized with reference to FIGS. 1-8 to implement the techniques described herein.

FIG. 12 shows an example genome-wide pattern of RNA expression as assigned by cell type.

FIG. 13 shows an example of SPN abundance in relation to CAP scores.

FIG. 14 shows an example comparing HTT expression.

FIG. 15 shows an example of CAG measurement correlations.

FIGS. 16A and 16B show an example of cell-type specificity of the CAG repeat length in Huntington's disease.

FIG. 17 shows an example of comparing HTT CAG repeat length and gene expression in SPNs.

FIG. 18 shows an example demonstrating consistency of long repeat expansion-associated gene expression changes across individual persons with Huntington's disease.

FIG. 19 shows an example of continuously escalating gene expression distortion beyond 150 CAG repeat lengths.

FIG. 20 shows an example of median fold change plots quantifying upregulated and downregulated genes for a plurality of individual persons with Huntington's disease.

FIG. 21 shows an example of de-repression in genes having long CAG repeat expansions.

FIG. 22 shows an example analysis of transcriptional changes in relation to CAP score.

FIG. 23 shows an example schematic of a hypothesized model for post-mitotic repeat expansion.

FIG. 24 shows an example of modeling data for repeat expansion dynamics.

FIG. 25 shows an overview of a model for neuropathology in HD.

### DETAILED DESCRIPTION

### Overview

Repeat expansion disorders are a group of genetic disorders that include repetitive sequences of nucleotides within specific genes being abnormally expanded. These repetitive sequences, which typically comprise three to six nucleotides repeated multiple times, are found throughout the human genome and play roles in normal cellular functions. However, when these repetitive sequences expand beyond a certain threshold, they can lead to dysfunction of the associated gene products (e.g., proteins) and contribute to the development of debilitating diseases. Examples of repeat expansion disorders include Huntington's disease, fragile X syndrome, myotonic dystrophy, and several types of spinocerebellar ataxias. Repeat expansion disorders often affect the nervous system and can lead to a wide range of symptoms, including cognitive impairment, movement disorders, muscle weakness, and developmental delays.

Repeat expansion disorders exhibit several common features, including somatic instability. Somatic instability refers to the dynamic nature of repeat expansions, where the number of repeats can change or expand further within an affected individual's lifetime, particularly in non-germline (e.g., somatic) tissues. This somatic instability can result in mosaicism, where different cells within the individual's body have varying lengths of the repetitive sequence.

Given the complex nature of repeat expansion disorders and the challenges associated with their diagnosis and treatment, techniques that enable the lengths of the repetitive sequence to be quantified may aid an understanding of disease pathogenesis and progression as well as therapeutic efficacy. However, the mosaic nature makes it difficult to accurately determine a distribution of repeat sequence lengths of the repetitive sequence in a biological sample (e.g., a sample of cells). For example, conventional techniques that rely on bulk amplification of nucleic acid from the biological sample and subsequent sequencing may introduce distorting effects that bias toward shorter length molecules, causing shorter molecules to be over-represented (relative to their representation in the original biological sample) and longer molecules to be under-represented (relative to their representation in the original biological sample) in the final, amplified molecular mixture that is analyzed. Moreover, these techniques do not enable analysis at single-cell resolution, which would enable changes in gene expression to be correlated with the length of the repetitive sequence as well as disease processes that occur in certain specific cell types to be measured.

To overcome these issues, methods and compositions for analysis and treatment of repeat expansion disorders are disclosed herein. In accordance with the described techniques, a targeted variable length sequence repeat region may be amplified from nucleic acid extracted from a biological sample comprising a plurality of cells using primers that incorporate molecular labels to uniquely label amplicons generated from individual nucleic acid molecules of the biological sample. The uniquely labeled amplicons may be sequenced, and sequences of the molecular labels enable sequencing read families (e.g., sets of reads derived from the same nucleic acid molecule in the biological sample or generated at an earlier stage of amplification) to be identified in the sequencing data. By way of example, the sequencing read families group sequencing reads identified for the individual nucleic acid molecules based on the unique sequences of the molecular labels. The individual nucleic acid molecules may be RNA transcripts or genomic DNA molecules, for instance. Moreover, the molecular labels may further include cell barcodes than enable a cell of origin of the biological sample to be identified.

For example, although there may be more amplicons generated (and thus more reads generated) for shorter lengths of the variable length sequence repeat region, the molecular labels enable a single consensus sequence (e.g., a nucleic acid molecule-specific consensus sequence) to be generated for each read family, which neutralizes the distorting effects of the amplification. Accordingly, a sequence repeat length distribution generated from a plurality of nucleic acid molecule-specific consensus sequences produces a more accurate measure of the distribution of sequence repeat lengths of the biological sample compared to conventional techniques, even across a wide range of sequence repeat lengths. As a result, the somatic instability of genes involved in repeat expansion disorders may be accurately investigated, which may inform on disease progression, treatment efficacy, underlying pathogenic mechanisms, and so forth.

In at least one implementation, true sequences of variable repeat sequences in single cell types from a subject can be determined. The variable repeat sequences can be any sequence in a subject that is subject to somatic expansion or somatic mutation (as used herein, “somatic alteration”). In example implementations, somatic alterations in a subject do not occur in every cell type or every cell of a cell type. In example implementations, it is beneficial to identify subjects having a specific somatic alteration in any cell in the subject (e.g., to determine disease progression or to study the disease). In example implementations, it is beneficial to identify the specific compilation of somatic alterations in any or all cells in the subject (e.g., to determine disease progression or to study the disease). In example implementations, the methods disclosed herein are applicable to any disease having a somatic alteration that is variable between cells in a subject. By way of example, the disease is a repeat expansion disorder gene. More than forty diseases, most of which primarily affect the nervous system, are caused by expansions of simple sequence repeats dispersed throughout the human genome. In example implementations, accurate diagnosis, with knowledge of repeat length in the affected cell types, is beneficial in the management of these diseases. The current methods allow the ability to identify a true consensus sequence for the region having the somatic alteration in affected cells in a subject. In example implementations, consensus sequences for a variable repeat sequence length are determined (e.g., consensus sequence lengths for more than one cell type and more than one cell of each cell type).

In some aspects, the techniques described herein relate to a method including: generating labeled amplicons of a variable repeat region of a gene, said generating using primers that introduce at least one molecular label to respective nucleic acid molecules of origin of a biological sample; sequencing the labeled amplicons to generate sequencing reads having the at least one molecular label incorporated; and generating a sequence repeat length distribution of the variable repeat region in at least a portion of the biological sample based on the sequencing reads.

In some aspects, the techniques described herein relate to a method, wherein generating the sequence repeat length distribution of the variable repeat region in at least the portion of the biological sample based on the sequencing reads includes: identifying read families based on the at least one molecular label, each read family including a subset of the sequencing reads having a matched sequence for the at least one molecular label; generating molecule-specific consensus sequences based on the identified read families, each molecule-specific consensus sequence corresponding to a sequence of a single nucleic acid molecule of origin of the biological sample; determining consensus repeat lengths for respective molecule-specific consensus sequences; and generating the sequence repeat length distribution based on the consensus repeat lengths.

In some aspects, the techniques described herein relate to a method, wherein the labeled amplicons include cDNA of the variable repeat region, and wherein the at least one molecular label includes a unique molecular identifier having a sequence that varies based on an RNA transcript of origin of the labeled amplicons.

In some aspects, the techniques described herein relate to a method, wherein the at least one molecular label further includes a cell barcode that varies based on a cell of origin of the labeled amplicons in the biological sample.

In some aspects, the techniques described herein relate to a method, wherein the at least one molecular label further includes at least one index sequence that is specific to the biological sample.

In some aspects, the techniques described herein relate to a method, wherein the primers that introduce the at least one molecular label to the respective nucleic acid molecules of origin are used during a reverse transcription reaction, and the method further includes amplifying the labeled amplicons during a transcriptome amplification reaction.

In some aspects, the techniques described herein relate to a method, wherein the transcriptome amplification reaction uses spike-in primers targeting the variable repeat region of the gene.

In some aspects, the techniques described herein relate to a method, wherein the method further includes enriching the amplified labeled amplicons for the variable repeat region of the gene during a targeted amplification reaction.

In some aspects, the techniques described herein relate to a method, wherein the targeted amplification reaction uses gene-specific primers for the variable repeat region of the gene, at least one of the gene-specific primers including an affinity purification tag at a 5′ end.

In some aspects, the techniques described herein relate to a method, wherein the respective nucleic acid molecules are molecules of genomic DNA, and the primers that introduce the at least one molecular label to the respective nucleic acid molecules of origin are used during a first amplification reaction having a first number of reaction cycles, and the method further includes: amplifying the labeled amplicons during a second amplification reaction having a second number of reaction cycles that is greater than the first number of reaction cycles.

In some aspects, the techniques described herein relate to a method, wherein the gene is associated with a repeat expansion disorder, and wherein the portion of the biological sample is defined by a type of cell.

In some aspects, the techniques described herein relate to a system including: a sequencing data processor executing instructions stored in a non-transitory computer-readable storage medium and configured to: receive sequencing data including sequencing reads of labeled amplicons of a variable length sequence repeat region, the labeled amplicons having molecular labels uniquely identifying individual nucleic acid molecules of origin from a biological sample; and generate a sequence repeat length distribution of the variable length sequence repeat region in the biological sample based on the sequencing data.

In some aspects, the techniques described herein relate to a system, wherein to generate the sequence repeat length distribution of the variable length sequence repeat region in the biological sample based on the sequencing data, the sequencing data processor is further configured to: identify sequences of the molecular labels in the sequencing reads; group the sequencing reads into read families based on the sequences of the molecular labels, wherein each read family includes a subset of the sequencing reads that has a matching sequence for at least one of the molecular labels; and determine a consensus repeat length for respective read families.

In some aspects, the techniques described herein relate to a system, wherein the subset of the sequencing reads in each read family corresponds to an individual nucleic acid molecule of origin from the biological sample.

In some aspects, the techniques described herein relate to a system, wherein the sequence repeat length distribution indicates a frequency of individual sequence repeat lengths of the variable length sequence repeat region and a range of sequence repeat lengths of the variable length sequence repeat region in the biological sample, and the sequencing data processor is further configured to: simulate repeat expansion dynamics based on the sequence repeat length distribution; and generate an expansion dynamics model of an associated repeat expansion disorder of the variable length sequence repeat region.

In some aspects, the techniques described herein relate to a system, wherein the molecular labels are introduced via a reverse transcription reaction using primers targeting RNA transcripts, and wherein the individual nucleic acid molecules of origin include the RNA transcripts.

In some aspects, the techniques described herein relate to a method including: generating, via a reverse transcription reaction, labeled amplicons of a targeted variable length sequence repeat region of RNA transcripts from a biological sample, the reverse transcription reaction introducing molecular labels that uniquely label the labeled amplicons derived from individual RNA transcripts of individual nuclei of the biological sample; preparing, via at least one amplification reaction and at least one purification process, the labeled amplicons for sequencing; and determining, via the sequencing of the labeled amplicons, sequence repeat lengths of the targeted variable length sequence repeat region of the individual RNA transcripts.

In some aspects, the techniques described herein relate to a method, further including generating, based on the determined sequence repeat lengths, a sequence repeat length distribution of the targeted variable length sequence repeat region of the individual RNA transcripts in a per-cell basis.

In some aspects, the techniques described herein relate to a method, wherein the molecular labels include a unique molecular label sequence that distinguishes the labeled amplicons derived from the individual RNA transcripts of a single nucleus from each other and a cell barcode sequence that distinguishes the labeled amplicons derived from different nuclei.

In some aspects, the techniques described herein relate to a method, wherein, determining, via the sequencing of the labeled amplicons, the sequence repeat lengths of the targeted variable length sequence repeat region of the individual RNA transcripts from the biological sample includes: generating sequencing data via the sequencing, the sequencing data including sequencing reads of the labeled amplicons; identifying read families based on the cell barcode sequence and the unique molecular label sequence in the sequencing reads, each read family including a matched sequence for the cell barcode sequence and the unique molecular label sequence; and determining the sequence repeat lengths of the targeted variable length sequence repeat region of the individual RNA transcripts based on respective sequence repeat length distributions of the read families.

In some aspects, the techniques described herein relate to a method including: generating labeled amplicons of a variable repeat region of genomic DNA obtained from a biological sample, said generating using primers that introduce at least one molecular label to respective DNA molecules of origin of the genomic DNA; sequencing the labeled amplicons to generate sequencing reads having the at least one molecular label incorporated; and generating a sequence repeat length distribution of the variable repeat region in the biological sample based on the sequencing reads.

In some aspects, the techniques described herein relate to a method, wherein generating the sequence repeat length distribution of the variable repeat region in the biological sample based on the sequencing reads includes: identifying read families based on the at least one molecular label, each read family including a subset of the sequencing reads having a matched sequence for the at least one molecular label; generating molecule-specific consensus sequences based on the identified read families, each molecule-specific consensus sequence corresponding to a sequence of a single DNA molecule of origin of the genomic DNA; determining consensus repeat lengths for respective molecule-specific consensus sequences; and generating the sequence repeat length distribution based on the consensus repeat lengths.

In some aspects, the techniques described herein relate to a method, wherein the labeled amplicons include a first molecular label at a first position flanking the variable repeat region, and wherein a first molecular label sequence of the first molecular label varies based on a DNA molecule of origin of the labeled amplicons.

In some aspects, the techniques described herein relate to a method, wherein the labeled amplicons further include a second molecular label at a second position flanking the variable repeat region, the second position at an opposite end of the variable repeat region from the first position, and wherein a second molecular label sequence of the second molecular label varies based on the DNA molecule of origin of the labeled amplicons.

In some aspects, the techniques described herein relate to a method, wherein the primers that introduce the at least one molecular label to the respective DNA molecules are used during a first amplification reaction having a first number of reaction cycles, and the method further includes: amplifying the labeled amplicons during a second amplification reaction having a second number of reaction cycles that is greater than the first number of reaction cycles.

In some aspects, the techniques described herein relate to a method, wherein the first number of reaction cycles is in a first range between one and five, and wherein the second number of reaction cycles is in a second range between six and forty.

In some aspects, the techniques described herein relate to a method, wherein amplification primers used for the second amplification reaction introduce one or both of indices and sequencing adapters for the sequencing.

In some aspects, the techniques described herein relate to a method, wherein the primers include: forward labeling primers having a first locus-specific sequence targeting an upstream region of the variable repeat region, each forward labeling primer molecule of the forward labeling primers having a different forward primer molecular label sequence with respect to each other; and reverse labeling primers having a second locus-specific sequence targeting a downstream region of the variable repeat region, each reverse labeling primer molecule of the reverse labeling primers having a different reverse primer molecular label sequence with respect to each other.

In some aspects, the techniques described herein relate to a method, wherein the first locus-specific sequence is positioned at a 3′ end of the forward labeling primers, and the forward labeling primers further include a forward tag sequence at a 5′ end for further amplification using a forward amplification primer, said forward amplification primer configured to anneal to the forward tag sequence.

In some aspects, the techniques described herein relate to a method, wherein the second locus-specific sequence is positioned at a 3′ end of the reverse labeling primers, and the reverse labeling primers further include a reverse tag sequence at a 5′ end for further amplification using a reverse amplification primer, said reverse amplification primer configured to anneal to the reverse tag sequence.

In some aspects, the techniques described herein relate to a method, wherein the variable repeat region is within a gene associated with a repeat expansion disorder.

In some aspects, the techniques described herein relate to a system including: a sequencing data processor executing instructions stored in a non-transitory computer-readable storage medium and configured to: receive sequencing data including sequencing reads of labeled amplicons of a variable length sequence repeat region, the labeled amplicons having molecular labels uniquely identifying individual DNA molecules of origin from a biological sample; and generate a sequence repeat length distribution of the variable length sequence repeat region in the biological sample based on the sequencing data.

In some aspects, the techniques described herein relate to a system, wherein to generate the sequence repeat length distribution of the variable length sequence repeat region in the biological sample based on the sequencing data, the sequencing data processor is further configured to: identify sequences of the molecular labels in the sequencing reads; group the sequencing reads into read families based on the sequences of the molecular labels, wherein each read family includes a subset of the sequencing reads that has a matching sequence for at least one of the molecular labels; and determine a consensus repeat length for respective read families.

In some aspects, the techniques described herein relate to a system, wherein the subset of the sequencing reads in each read family corresponds to an individual DNA molecule of origin from the biological sample.

In some aspects, the techniques described herein relate to a system, wherein the sequence repeat length distribution indicates a frequency of individual sequence repeat lengths of the variable length sequence repeat region and a range of sequence repeat lengths of the variable length sequence repeat region in the biological sample.

In some aspects, the techniques described herein relate to a system, wherein the molecular labels are introduced via at least one amplification reaction using primers targeting the variable length sequence repeat region of genomic DNA, and wherein the individual DNA molecules of origin include the genomic DNA.

In some aspects, the techniques described herein relate to a method including: generating, via a first amplification reaction, labeled amplicons of a targeted variable length sequence repeat region from a bulk sample of genomic DNA, the first amplification reaction introducing labeling primers that uniquely label the labeled amplicons derived from individual DNA molecules of the bulk sample of genomic DNA; amplifying, via a second amplification reaction, the labeled amplicons for sequencing; and determining, via the sequencing of the labeled amplicons, sequence repeat lengths of the targeted variable length sequence repeat region of the individual DNA molecules in the bulk sample of the genomic DNA.

In some aspects, the techniques described herein relate to a method, further including generating, based on the determined sequence repeat lengths, a sequence repeat length distribution of the targeted variable length sequence repeat region of the individual DNA molecules in the bulk sample of the genomic DNA.

In some aspects, the techniques described herein relate to a method, wherein the labeling primers incorporate at least one unique molecular label sequence into the labeled amplicons derived from the individual DNA molecules of the bulk sample of the genomic DNA.

In some aspects, the techniques described herein relate to a method, wherein, determining, via the sequencing of the labeled amplicons, the sequence repeat lengths of the targeted variable length sequence repeat region of the individual DNA molecules in the bulk sample of the genomic DNA includes: generating sequencing data via the sequencing, the sequencing data including sequencing reads of the labeled amplicons; identifying read families based on the at least one unique molecular label sequence in the sequencing reads, each read family including a matched sequence for the at least one unique molecular label sequence; and determining the sequence repeat lengths of the targeted variable length sequence repeat region of the individual DNA molecules in the bulk sample of the genomic DNA based on respective sequence repeat length distributions of the read families.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

As used herein, the singular forms “a”, “an”, and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less from the specified value, insofar as such variations are appropriate to perform in the disclosed technique. It is to be understood that the value to which the modifier “about” or “approximately” refers is itself also disclosed.

As used herein, a “biological sample” may contain whole cells, live cells, cell nuclei, and/or cell debris. The biological sample may contain (or be derived from) a bodily fluid, which may refer to any fluid that is naturally produced by and/or circulates within the body of an organism, and/or bodily tissue. Non-limiting examples of bodily fluids include bile, blood, plasma, urine, cerebrospinal fluid, saliva, lymph fluid, sweat, synovial fluid, and mixtures of one or more thereof. Non-limiting examples of bodily tissues include brain tissue, liver tissue, and muscle tissue. Bodily fluids and/or bodily tissue may be obtained from a mammal organism, for example by puncture, or other collecting or sampling procedures. Biological samples include in vivo and ex vivo samples obtained from a biological entity (e.g., cells, tissues, bodily fluids and their progeny) and/or in vitro samples, such as cell cultures.

The terms “subject,” “individual,” and “patient” are used interchangeably herein to refer to an organism that serves as the biological entity. Example organisms include, but are not limited to, mammals such as murines, simians, humans, farm animals, sport animals, and pets.

Various implementations are described hereinafter. It should be noted that the specific implementations are not intended as an exhaustive description or as a limitation to the broader aspects discussed herein. One aspect described in conjunction with a particular implementation is not necessarily limited to that implementation and can be practiced with any other implementation(s). Reference throughout this specification to “one implementation”, “an implementation,” “an example implementation,” means that a particular feature, structure or characteristic described in connection with the implementation is included in at least one implementation of the present invention. Thus, appearances of the phrases “in one implementation,” “in an implementation,” or “an example implementation” in various places throughout this specification are not necessarily all referring to the same implementation, but may. Furthermore, the particular features, structures or characteristics may be combined in any suitable manner, as would be apparent to a person skilled in the art from this disclosure, in one or more implementations. Furthermore, while some implementations described herein include some but not other features included in other implementations, combinations of features of different implementations are meant to be within the scope of the invention. For example, in the appended claims, any of the claimed implementations can be used in any combination.

### Example Environment

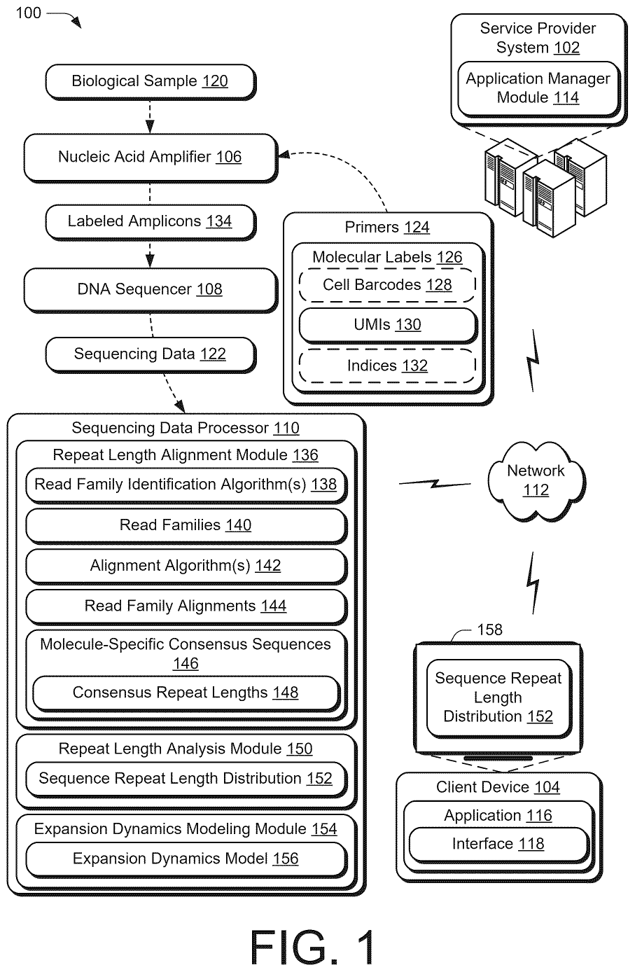

FIG. 1 is an illustration of an environment 100 in an example implementation that is operable to employ sequence repeat length distribution analysis as described herein. The illustrated environment 100 includes a service provider system 102, a client device 104, a nucleic acid amplifier 106, a DNA sequencer 108, and a sequencing data processor 110 that are communicatively coupled, one to another, via a network 112. Although the sequencing data processor 110 is illustrated as separate from the service provider system 102, the client device 104, and the DNA sequencer 108, this functionality may be incorporated as part of the service provider system 102, the client device 104, and/or the DNA sequencer 108, further divided among other entities, and so forth. By way of example, an entirety of or portions of the functionality of the sequencing data processor 110 may be incorporated as part of the DNA sequencer 108 and/or the client device 104. Additionally or alternatively, an entirety of or portions of the client device 104 may be incorporated as part of the DNA sequencer 108 and/or the sequencing data processor 110. Moreover, in at least one variation, the nucleic acid amplifier 106 and/or the DNA sequencer 108 is not communicatively coupled to the network 112.

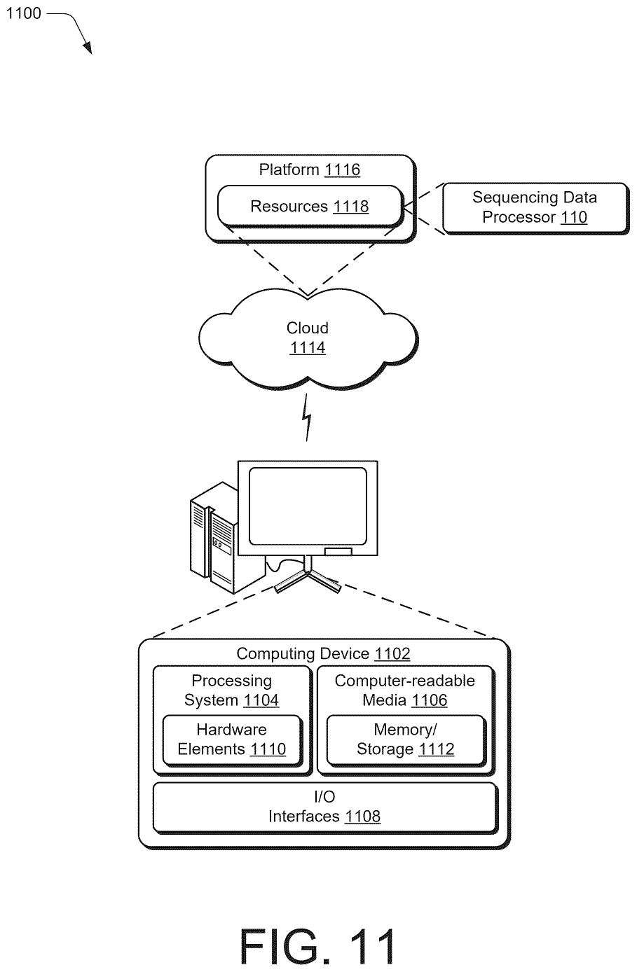

Computing devices that are usable to implement the service provider system 102, the client device 104, and the sequencing data processor 110 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 11.

The service provider system 102 is illustrated as including an application manager module 114 that is representative of functionality to provide access to the sequencing data processor 110 to a user of the client device 104 via the network 112. The application manager module 114, for instance, may expose content or functionality of the sequencing data processor 110 that is accessible via the network 112 by an application 116 of the client device 104. The application 116 may be configured as a network-enabled application, a browser, a native application, and so on, that exchanges data with the service provider system 102 via the network 112. The data can be employed by the application 116 to enable the user of the client device 104 to communicate with the service provider system 102, such as to receive application updates and features when the service provider system 102 provides functionality to manage the application 116.

In the context of the described techniques, the application 116 includes functionality to analyze data generated by a sequencing event to determine a sequence repeat length distribution thereof. In the illustrated example, the application 116 includes an interface 118 that is implemented at least partially in hardware of the client device 104 for facilitating communication between the client device 104 and the sequencing data processor 110. By way of example, the interface 118 includes functionality to receive inputs to the sequencing data processor 110 from the client device 104 (e.g., from a user of the client device 104) and output information, data, and so forth from the sequencing data processor 110 to the client device 104, as will be further elaborated herein.

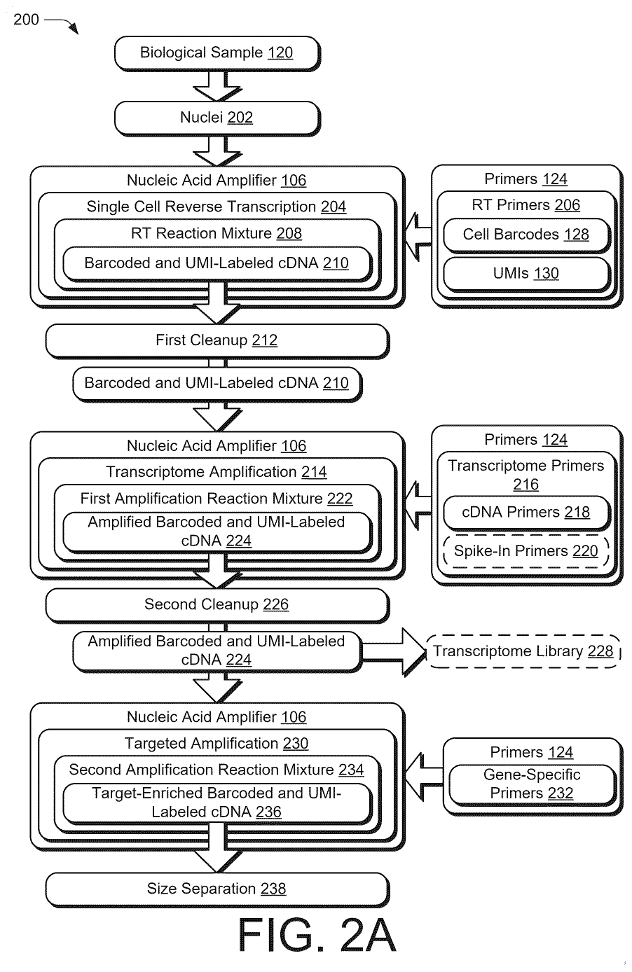

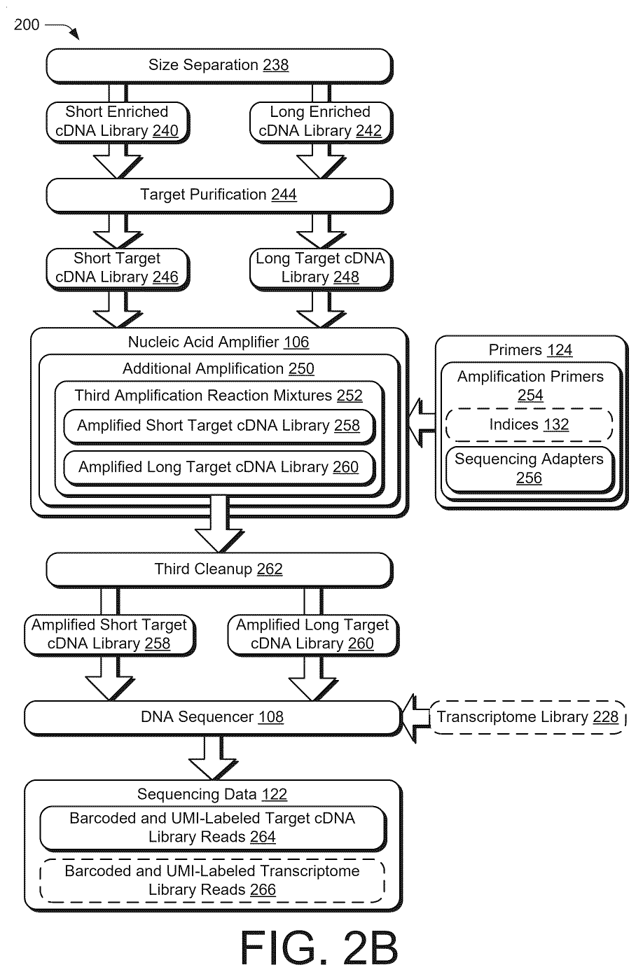

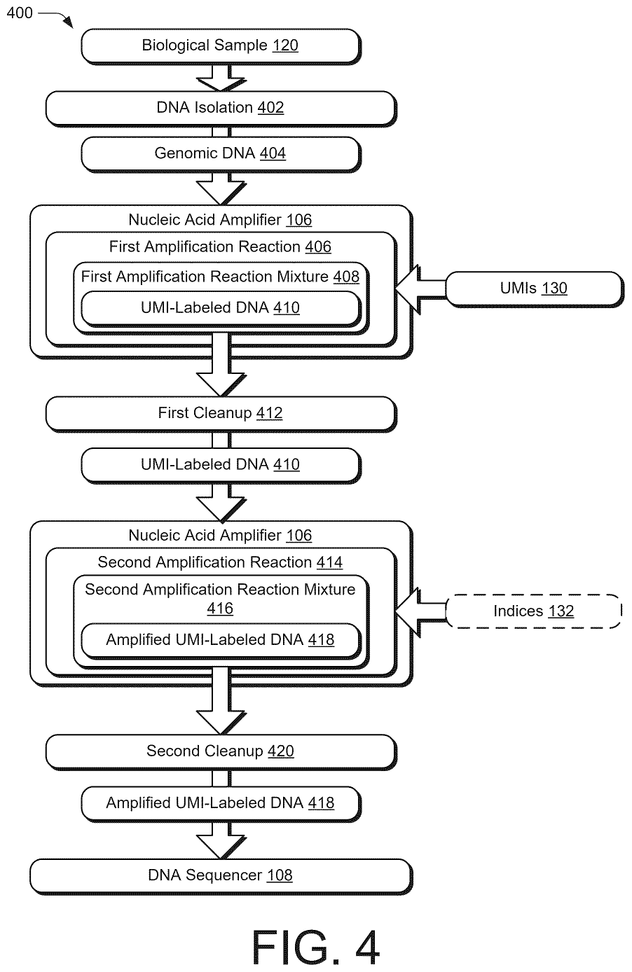

The sequencing event includes determining an order of nucleotides (e.g., adenine, thymine or uracil, cytosine, and guanine) in a sample of nucleic acid derived from a biological sample 120. The order of nucleotides is referred to herein as a “sequence.” The nucleotides are also referred to as “bases.” In at least one implementation, the nucleic acid comprises complementary DNA (cDNA) derived from ribonucleic acid (RNA) transcripts, such as described in detail with respect to FIGS. 2A-3 and 9. In at least one variation, the nucleic acid comprises amplified portions of genomic DNA, such as described in detail with respect to FIGS. 4, 5, and 10. However, it is to be appreciated that the techniques described herein may be adapted for sequencing other types of nucleic acids.

The DNA sequencer 108 is configured to produce sequencing data 122 that is analyzed by the sequencing data processor 110 to determine the order of nucleotides in the biological sample 120 of a portion thereof. In at least one implementation, the sequencing data 122 comprise a text-based file format, such as FASTQ files that store both nucleotide sequence information and quality scores for the bases in a sequencing read. In variations, the sequencing data 122 comprise another type of file format. The DNA sequencer 108 may use one of a plurality of sequencing techniques to produce the sequencing data 122, e.g., “sequencing reads” or “reads.” A read is an inferred sequence of base pairs (or base pair probabilities) corresponding to all or part of a single DNA fragment, for instance.

A typical sequencing experiment involves fragmentation of genomic DNA into millions of molecules, which may be selectively or non-selectively amplified, or generating cDNA fragments. In at least one implementation, the fragments (e.g., of genomic DNA or cDNA) are size-selected and appended with sequencing adapters to generate a sequencing library, which is sequenced to produce a set of reads. A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags, such as will be elaborated herein.

By way of example, the DNA sequencer 108 may use a short read sequencing technique that produces sequence fragments typically ranging from approximately 10 bases to approximately 500 bases and more typically from approximately 50 bases to approximately 800 bases. Sequence fragments produced via short read sequencing techniques are also referred to as “short reads.” Alternatively, the DNA sequencer 108 may use a long read sequencing technique that produces sequence fragments that typically range from 500 bases to 1,000,000 bases in length. Sequence fragments produced via long read sequencing techniques are also referred to as “long reads.”

As a non-limiting example, the DNA sequencer 108 utilizes high-throughput (e.g., “next-generation”) technologies to generate the sequencing data 122, e.g., the sequencing reads. In at least one implementation, the library members (e.g., genomic DNA or cDNA) may include sequencing adaptors that are compatible with use in, e.g., a reversible terminator method, long read nanopore sequencing, a pyrosequencing method, sequencing by ligation, ion torrent sequencing, single-molecule real-time (SMRT) sequencing, and the like. Due to the longer read length of long read sequencing, in at least one implementation, long read sequencing is used in order to generate a full-length sequence of a given library member in a single read.

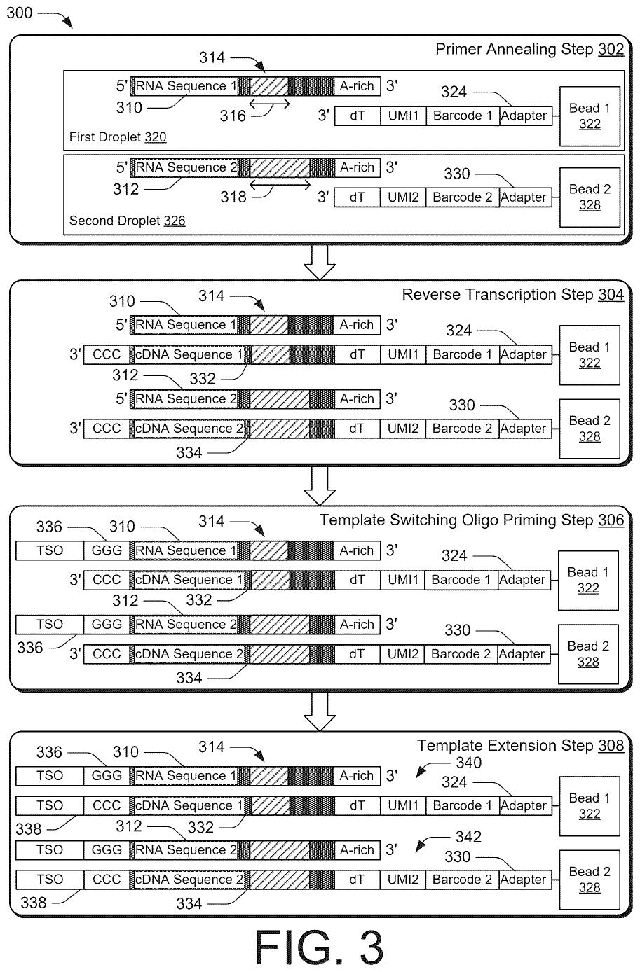

Regardless of the sequencing technique, in the illustrated example environment 100, the DNA sequencer 108 produces the sequencing data 122 for nucleic acid that has undergone labeling and amplification. In at least one implementation, nucleic acid isolated from the biological sample 120 is prepared for sequencing via reactions performed at the nucleic acid amplifier 106. The nucleic acid amplifier 106 is an instrument that facilitates cDNA synthesis through a reverse transcription reaction and/or DNA amplification (e.g., of the cDNA or genomic DNA) through an amplification reaction. By way of example, the nucleic acid amplifier 106 may be a thermal cycler having functionality to cycle through different temperature stages, which allow for the denaturation (e.g., separating double-stranded DNA into single strands or disrupting RNA secondary structure), annealing of primers 124 (e.g., short DNA sequences that bind to a target portion of the DNA or RNA), and extension of new, complementary strands of DNA from the primers 124 using an enzyme (e.g., a reverse transcriptase, a DNA polymerase, or engineered versions thereof). The nucleic acid amplifier 106, for instance, includes a thermal block or heating/cooling element to regulate temperature, a programmable interface to set cycling parameters (e.g., temperature and time), and heating/cooling mechanisms to rapidly transition between the different temperature stages. An overview of the reverse transcription reaction will be described herein with respect to FIG. 3. In at least one implementation, the reverse transcription reaction and the amplification reaction are performed in different nucleic acid amplifiers 106. Alternatively, the reverse transcription reaction and the amplification reaction are performed in the same nucleic acid amplifier 106.

In at least one implementation, the amplification reaction is a polymerase chain reaction (PCR), although other nucleic acid amplification techniques may be used. As used herein, “PCR” may include derivative forms of the reaction, including but not limited to reverse transcription (RT)-PCR, real-time PCR, nested PCR, quantitative PCR, multiplexed PCR, digital PCR, and assembly PCR. The amplification reaction may be performed in one or more rounds, also referred to herein as “reaction cycles.” A reaction cycle, for instance, may include a denaturation step followed by a primer annealing step, which is followed by an extension step. In at least one implementation, a PCR program may include an additional enzyme activation step prior to a first reaction cycle and an additional extension step after a final reaction cycle.

In one or more implementations, the primers 124 include a plurality of primer types, including reverse transcription primers used in a reverse transcription reaction (when used), gene-specific primers used in a targeted amplification reaction, and indexing primers (when used). In the example depicted in FIG. 1, the primers 124 include molecular labels 126. The molecular labels 126, for instance, are configured to uniquely label products arising from a specific nucleic acid (e.g., RNA or DNA) molecule and/or cell. By way of example, the molecular labels 126 include one or more or each of cell barcodes 128, unique molecular identifiers (UMIs) 130, and indices 132.

When included, the cell barcodes 128 may be used to identify a cell (e.g., nuclei) of origin of a nucleic acid sample, such as may be used in single cell/nucleus sequencing implementations. It is to be appreciated that the terms “cell” and “nucleus” may be used interchangeably herein to denote genetic material that arises from a single cell of origin. By way of example, a given cell may include a single nucleus. As such, the cell barcodes 128 correspond to nuclei barcodes. The term “barcode” as used herein refers to a short sequence of nucleotides (for example, DNA or RNA) that is used as an identifier of the source of an associated molecule, such as a cell-of-origin. A barcode may be a unique, non-naturally occurring nucleic acid sequence, for instance. The cell barcodes 128 may have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Nucleic acids can be labeled with multiple nucleic acid barcodes in a combinatorial fashion, such as by using a barcode concatemer.

The UMIs 130 may be short sequences of random nucleotides (e.g., typically ranging from 8-12 bases in length, or from 4-20 bases in length) that are configured to uniquely identify amplification products derived from individual molecules of origin in the biological sample 120. By way of example, the UMI 130 may be a sequencing linker or a subtype of nucleic acid that enables unique amplified products to be quantified. In at least one implementation, a single UMIs 130 or pair of UMIs 130 is added to a particular nucleic acid, and each amplicon generated from that nucleic acid will have the same single UMIs 130 or pair of UMIs 130, as will be elaborated herein. In example implementations, the cell barcodes 128 and/or the UMIs 130 are used to identify the source of each nucleic acid sequenced. For example, the cell barcodes 128, when included, may be used to identify a cell of origin of a sequencing read. The one or more UMIs 130 may be used to identify an individual nucleic acid molecule of origin of the sequencing read, which may be further linked to the cell of origin (e.g., when the cell barcodes 128 are used).

When used, the indices 132 may enable the sequencing event to be multiplexed. The indices 132 include a plurality of known index sequences of short (e.g., 8-12 nucleotide) sequences that are assigned to a given sample to be sequenced. One index or a pair of indices 132 may be used. The indices 132 may be introduced through primers 124 that target common regions of primers 124 used in a previous reverse transcription or amplification reaction. As such, DNA molecules (e.g., cDNA or genomic DNA) derived from the same sample (e.g., the same biological sample 120) may have the same index or indices 132, and DNA molecules derived from different samples may have different indices 132, thus enabling sequencing data 122 corresponding to one sample to be distinguished from another. In variations, however, the indices 132 may be omitted, such as when multiplexing is not used for sequencing. Additionally, or alternatively, the indices 132 may be introduced via another technique, such as adapter ligation, rather than via the primers 124.

Thus, the molecular labels 126 include the UMIs 130 and optionally further include the cell barcodes 128 and/or the indices 132, depending on a particular type of experiment being performed. The cell barcodes 128, the UMIs 130, and/or the indices 132 may be introduced via separate processes with respect to each other, examples of which will be further described below.

In the context of reverse transcription, the primers 124 include an RNA-targeting primer, such as a sequence of deoxythymidine (dT) nucleotides (also referred to herein as an “oligo dT”). The oligo dT is configured to anneal to the 3′ polyadenylation (polyA) tail of mRNA molecules through complementary binding (e.g., base pairing through hydrogen bonding, where A pairs with T/U and C pairs with G). The primers 124 may further include a template switching oligo (TSO) primer that is configured to extend the 3′ end of the newly synthesized molecule of cDNA. By way of example, when the reverse transcriptase enzyme reaches the 5′ terminal end of the RNA, the reverse transcriptase enzyme may add additional nucleotides (e.g., a short sequence of Cs) to the 3′ end of the newly synthesized strand of the cDNA. These additional nucleotides may provide an annealing site of the TSO primer, and the reverse transcriptase enzyme switches template strands from the RNA to the TSO primer and continues synthesizing the cDNA to the 5′ end of the TSO primer. By doing so, the resulting cDNA includes an entirety of the information in the RNA. The primers 124 may further include one or more “spike-in” primers designed to target a gene transcript of a target sequence, e.g., such as the CAG repeat region of HTT. The addition of spike-in primers increases yield of the target sequence by making successful amplification independent of the (only partially efficient) standard, template-agnostic “template switch” step of reverse transcription. An overview of the reverse transcription process will be described below with respect to FIG. 3.

In the context of DNA amplification (e.g., of cDNA or genomic DNA), the primers 124 may include one or more forward primers configured to anneal to the “antisense” or “non-coding strand” of the denatured DNA through complementary binding (e.g., base pairing through hydrogen bonding, where A pairs with T and C pairs with G) with the antisense strand. The primers 124 further include one or more reverse primers configured to anneal to the “sense” or “coding” strand of the denatured DNA through complementary binding with the sense strand. During amplification, the forward primer serves as the starting point for DNA synthesis that is complementary to the non-coding strand, and the reverse primer serves as the starting point for DNA synthesis that is complementary to the coding strand. DNA synthesis by the polymerase enzyme extends from the primers 124 in opposite directions, resulting in the amplification of the DNA segment located between the two primers. As used herein, an “amplicon” is a newly synthesized portion of DNA targeted via the primers 124. During a targeted amplification reaction, for instance, a portion of DNA (e.g., cDNA or genomic DNA) comprising a variable length sequence repeat region of a gene of interest, such as the CAG repeat region of HTT, is enriched using gene-specific primers of the primers 124. The gene-specific primers are designed to amplify the portion of DNA comprising the variable length sequence repeat region through complementary binding of one of the denatured strands.

In at least one implementation, more than one process is performed at the nucleic acid amplifier 106. In an example implementation of single cell/nucleus sequencing, reverse transcription may be performed to introduce the cell barcodes 128 and the UMIs 130 in a manner that generally results in one UMI 130 or set (e.g., pair) of UMIs 130 being incorporated into cDNA generated from a given RNA transcript of the biological sample 120. By way of example, the cell barcodes 128 may be assigned per cell/nucleus whereas the UMIs 130 are unique for individual RNA transcripts of origin such that cDNA molecules derived from RNA transcripts of a single cell/nucleus have the same cell barcode 128 and different UMIs 130 with respect to each other. Furthermore, cDNA derived from RNA transcripts from different cells/nuclei have different cell barcodes 128. The cell barcodes 128 and/or the UMIs 130 may be introduced via one or more primers 124, for instance. A subsequent targeted amplification reaction may be performed in which a portion of the cDNA comprising a variable length sequence repeat region of a gene of interest, such as the CAG repeat region of HTT, is enriched using gene-specific primers of the primers 124, such as mentioned above. A more detailed overview of an example implementation of the single cell/nucleus sequencing is described below with respect to FIGS. 2A-3.

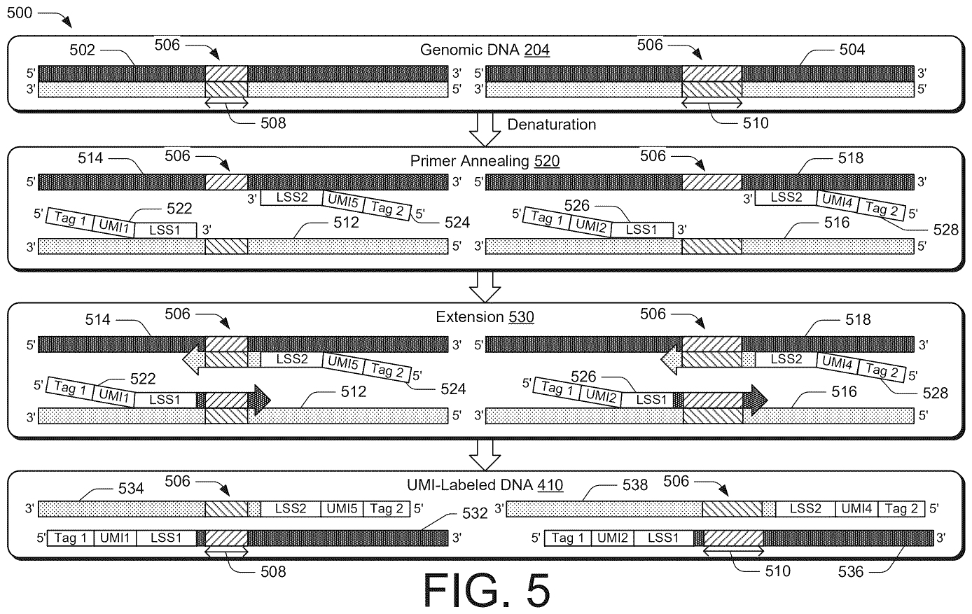

In an example implementation of genomic DNA sequencing, a first amplification reaction, also referred to herein as a first PCR, may be performed to introduce the UMIs 130 in a manner that generally results in one set of UMIs 130 being incorporated into an amplicon generated from a given molecule of DNA from the biological sample 120. Subsequently, a second amplification reaction, also referred herein to as a second PCR, may be performed to further amplify the amplicons labeled with the UMIs 130 and optionally introduce the indices 132. By way of example, forward primers used in the first amplification reaction may include one or more common regions with respect to each other (e.g., region(s) that are the same for the forward primers) and a variable region that includes a sequence that is specific to one forward primer as the UMI. Similarly, the reverse primers used in the first amplification reaction may include one or more common regions (e.g., region(s) that are the same for the reverse primers) and a variable region that includes a sequence that is specific to one reverse primer as the UMI. Examples of the primers 124 having common regions and the UMIs 130 as molecular labels 126 will be further described with respect to FIGS. 4 and 5. Using two UMIs 130 (e.g., one on the forward primer and one on the reverse primer) may enable recombinant/chimeric molecules and/or multiple successive priming events to be identified during a downstream computation analysis because such molecules share one of the two UMIs 130 in common, such as will be further elaborated herein.

During the first PCR, the primers 124 may also prime products of earlier PCR cycles (rather than just priming the DNA from the biological sample 120) in a process referred to herein as re-priming. As such, a first number of PCR cycles used in the first PCR is small. However, too few PCR cycles in the first PCR may result in some DNA from the biological sample 120 not being amplified and labeled, which can impose a problematic limit on data yield particularly when an amount of input DNA is low. As such, the first number of PCR cycles used in the first PCR may be adjusted based on the amount of input DNA, and subsequent computational analysis may be used to recognize re-priming events, as elaborated below.

In the example genomic DNA sequencing implementation, the indices 132, when included, may be introduced through primers 124 that target common regions of the primers 124 used in the first amplification reaction, thus ensuring that labeled amplicons, and not the genomic DNA, are further amplified and labeled with the indices 132. When the indices 132 are not included, the primers 124 used in the second amplification reaction may target the common regions of the forward and reverse primers used in the first amplification reaction without appending index sequences. A more detailed overview of an example implementation of the genomic DNA sequencing is described below with respect to FIGS. 4 and 5.

Labeled amplicons 134 (e.g., labeled with the UMIs 130 and optionally with the cell barcodes 128 and/or the indices 132) are generated via the one or more reverse transcription and/or amplification reactions mentioned above and sequenced via the DNA sequencer 108 to generate the sequencing data 122. The sequencing data processor 110 receives the sequencing data 122 and determines sequences (e.g., consensus sequences) of the nucleotides in the sample therefrom using a repeat length alignment module 136. In at least one implementation, the repeat length alignment module 136 includes one or more read family identification algorithms 138 for determining which reads correspond to a same nucleic acid molecule of origin in the biological sample 120. As mentioned above, the molecular labels 126 may be incorporated in a manner that generally results in one UMI 130 or pair of UMIs 130 (e.g., from one forward and one reverse primer in some genomic DNA implementations) being incorporated into amplicons generated from a given nucleic acid molecule from the biological sample 120. In single cell/nucleus sequencing implementations, the molecular labels 126 may further be incorporated such that amplicons generated from nucleic acid of a single cell/nucleus include a same cell barcode 128. Optionally, the molecular labels 126 may be incorporated such that amplicons of the same biological sample 120 include the same one or more indices 132. Thus, the one or more read family identification algorithms 138 may include statistical and/or computational analysis algorithm(s) and/or model(s) to identify read families 140 based on sequence(s) of the UMIs 130, alone or in combination with the cell barcodes 128 and the indices 132.

By way of example, a given read family of the read families 140 may comprise a subset of the sequencing data 122 (e.g., reads) that has the same UMI or pair of UMIs 130 (e.g., one forward primer UMI and one reverse primer UMI in example genomic DNA sequencing implementations). When cell barcodes 128 are used in single cell/nucleus RNA-seq applications, the read family may further comprise the same cell barcode 128. When the indices 132 are used for multiplexed sequencing, the read family may further comprise the same index or pair of indices 132 (e.g., one forward primer index and one reverse primer index). For instance, the one or more read family identification algorithms 138 may first sort the sequencing data 122 by sequences of the indices 132 (e.g., via index reads) to distinguish reads from one biological sample 120 from another biological sample 120 in a multiplexed sequencing reaction, when used. The sequencing data 122 for a given biological sample (e.g., a given index or pair of indices) may be further sorted based on the cell barcodes 128, when used, and the UMIs 130 so that sequencing reads having a common UMI sequence (or pair of UMI sequences) are grouped for further analysis.

For instance, the one or more read family identification algorithms 138 may identify sequences of the cell barcodes 128, the UMIs 130, and/or the indices 132 in the sequencing data 122 and group reads having a matching sequence (or sequences) into the read families 140 using fuzzy matching. Fuzzy matching takes into consideration substitution mutations that may be introduced during PCR and/or base read errors in the sequencing data 122 by allowing a configurable tolerance or threshold of mismatch (e.g., where a nucleotide position of one read varies with respect to another read due to a substitution, a deletion, or an insertion). As a non-limiting example, the threshold of mismatch may be one mismatched nucleotide. Additionally, the threshold of mismatch may be the same or different for different molecular labels 126. For instance, the cell barcodes 128, the UMIs 130, and/or the indices 132 may have different thresholds of mismatch with respect to each other.

Using the fuzzy matching described above, for instance, a first UMI sequence of a first read is considered to match a second UMI sequence of a second read in response to the first UMI sequence not exceeding the threshold of mismatch with respect to the second UMI sequence. Similarly, a first cell barcode sequence of the first read is considered to match a second cell barcode sequence of the second read in response to the first cell barcode sequence not exceeding the threshold of mismatch with respect to the second cell barcode sequence. As still another example, a first index sequence of the first read is considered to match a second index sequence of the second read in response to the first index sequence not exceeding the threshold of mismatch with respect to the second index sequence.

In addition to, or as an alternative to, fuzzy matching, the one or more read family identification algorithms 138 may employ at least one error correction technique to enhance an accuracy of the sequencing data 122. For instance, error correction may be applied to the sequencing data 122 prior to matching the molecular labels 126 and subsequently grouping the sequencing reads into the read families 140. Moreover, in at least one implementation, the one or more read family identification algorithms 138 may compare the sequences of the molecular labels 126 identified in the sequencing data 122 to an a priori known set of molecular label sequences as a part of the matching.

In at least one implementation where the molecular labels 126 include two UMIs 130, such as in some genomic DNA sequencing implementations, the one or more read family identification algorithms 138 transitively group reads that share at least one of the two UMIs 130 into the read families 140 using the fuzzy matching described above. Grouping reads that share at least one of the two UMIs 130 enables reads from recombinant/chimeric molecules and re-primed molecules to be included in the read families 140. By way of example, a chimeric molecule may result when an amplicon is incompletely made in one amplification cycle, and this incomplete amplicon then acts as a primer in a subsequent amplification cycle. The incomplete amplicon may include one UMI 130, for example. As another example, re-priming occurs when an amplicon that has already been labeled with a first pair of UMIs 130 is further amplified during the first amplification reaction, resulting in labeling with at least one different UMI 130. As a result of these processes, the sequencing data 122 may comprise reads having one UMI 130, more than two UMIs 130, or other deviations from an identified pair of UMIs 130 that are common to a given read family 140. Thus, the one or more read family identification algorithms 138 may at least initially group sequencing reads that share at least one UMI 130 sequence into the read families 140. This grouping by the one or more read family identification algorithms 138 may be transitive; for example, one or more reads with UMIs 130 having sequences A1 and B1 may be grouped into a read family with reads that have UMIs 130 having sequences A1 and B2, which may in turn be grouped into a read family with reads that have UMIs 130 A2 and B2.

The read families 140 are further analyzed via one or more alignment algorithms 142 of the repeat length alignment module 136. The one or more alignment algorithms 142 are configured to perform read alignment of the sequencing data 122 within the read families 140. In the context of sequencing the labeled amplicons 134, read alignment, also referred to simply as “alignment,” involves aligning (e.g., mapping) the reads in a given read family 140 to each other to generate read family alignments 144. By way of example, the one or more alignment algorithms 142 are representative of functionality for finding an alignment that increases (e.g., maximizes) a similarity between the reads of a given read family (e.g., reads having a common UMI or set of UMIs from a single sample and/or single cell of origin) using a scoring system that considers possible mismatches between the reads, e.g., due to mismatched bases that arise during amplification or base calling errors that arise during the sequencing. In this way, reads of the sequencing data 122 may be traced to a single DNA molecule (e.g., from a single cell) from the biological sample 120 based on the molecular labels 126.

The read family alignments 144 are used by the repeat length alignment module 136 to generate molecule-specific consensus sequences 146. By way of example, at respective positions in the read family alignments 144, the nucleotide present in the majority of read sequences may be chosen for the consensus sequence at that position. This process may involve counting the occurrences of each base at a specific position to determine which base is present in the majority of the read sequences. The molecule-specific consensus sequences 146 is a consensus sequence for a single DNA molecule of origin in the biological sample 120.

The repeat length alignment module 136 may further determine consensus repeat lengths 148 from the molecule-specific consensus sequences 146 and/or a sequence repeat length distribution of the corresponding read family 140. By way of example, respective consensus repeat lengths 148 correspond to a number of sequence repeats in a sequence repeat region that is expanded in a targeted repeat expansion disorder (e.g., targeted via the primers 124 and subsequent sequencing of the labeled amplicons 134) in a single DNA molecule of origin. In an example scenario where the CAG repeat region of HTT is targeted, the molecule-specific consensus sequences 146 indicate, as the consensus repeat lengths 148, lengths of the trinucleotide CAG repeat region for respective DNA molecules of origin. For instance, in this example scenario, a given consensus repeat length 148 is a number of CAG sequence repeat units in the variable CAG repeat region of HTT for a single DNA molecule extracted from the biological sample 120.

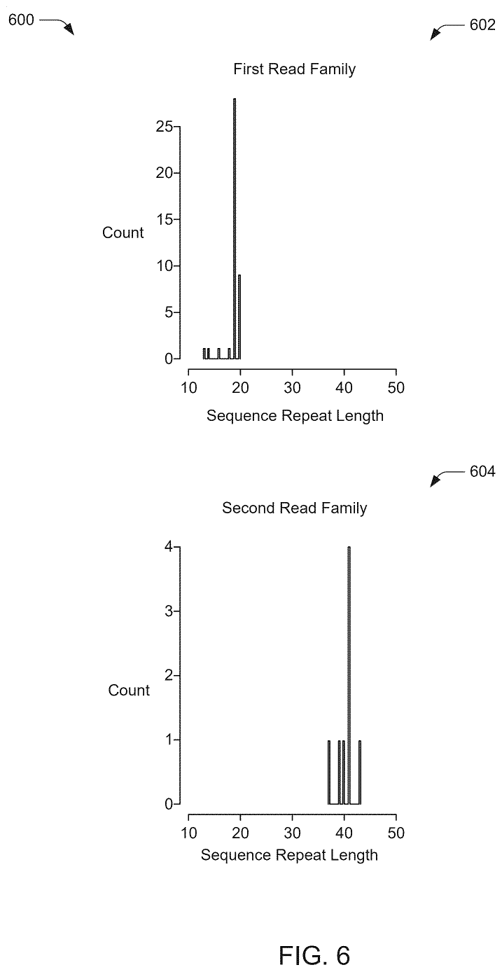

In at least one implementation, a range of sequence repeat lengths is represented in the reads of a given read family 140, resulting in a read family-specific sequence repeat length distribution 152. By way of example, sequence repeat length variability may arise from amplification “slippage” during the first amplification reaction or the second amplification reaction, which results in a new molecular sequence with a different repeat length than the sequence from which it is copied. In such scenarios, the consensus repeat length 148 may be a modal or median repeat length for the read family 140, such as will be further discussed with respect to FIG. 6.

In at least one implementation, the repeat length alignment module 136 may infer the consensus repeat lengths 148 by identifying a repeat region in the molecule-specific consensus sequences 146 that has a repetitive sequence of nucleotides without receiving specific user input as to the sequence repeat or a position of the repeat region in the molecule-specific consensus sequences 146. For instance, the repeat length alignment module 136 may identify, as the sequence repeat, a unit of nucleotides (e.g., a unit between one and six nucleotides in length, such as the trinucleotide CAG repeat of HTT) within the molecule-specific consensus sequences 146 that is consecutively repeated a plurality of times. In at least one variation, the repeat length alignment module 136 receives user input defining the sequence repeat (e.g., the unit of nucleotides that is repeated) and/or the position of the repeat region, such as based on expected sequence(s) flanking the repeat region.

In at least one implementation, the sequencing data processor 110 further includes a repeat length analysis module 150, which is representative of the functionality to evaluate the consensus repeat lengths 148 and generate a sequence repeat length distribution 152. The sequence repeat length distribution 152 indicates a range of sequence repeat lengths (e.g., from a minimum sequence repeat length value to a maximum sequence repeat length value) found in the biological sample 120 and a frequency of individual sequence repeat lengths within this range. By way of example, the DNA molecules of origin include sequence repeat regions of variable length due to the somatic instability of the sequence repeat region, and thus, the sequence repeat length distribution 152 indicates whether shorter or longer lengths occur more frequently. Referring again to the example scenario where the sequence repeat region of HTT is targeted, more advanced Huntington's disease may be indicated when the sequence repeat length distribution 152 includes longer average and/or median sequence repeat length values and/or the frequency of long sequence repeat lengths (e.g., longer than a threshold of interest) has increased. In at least one implementation, the sequence repeat length distribution 152 is usable in computational simulations or other more complex mathematical analyses that are configured to predict future disease progression (e.g., prognosis) or age of onset.

In at least one implementation, the sequencing data processor 110 further includes an expansion dynamics modeling module 154. The expansion dynamics modeling module 154 is representative of functionality to computationally model repeat expansion dynamics over a lifespan. The expansion dynamics modeling module 154 may analyze the sequence repeat length distribution 152 of a plurality of biological samples 120 in order to generate an expansion dynamics model 156. Subsequently, the expansion dynamics model 156 may be used to simulate and/or predict a disease progression of an individual based on the sequence repeat length of individual's inherited allele and the individual's age. This may give insight into the timing of somatic expansion of the sequence repeat region, for example. Alternatively, or in addition, the expansion dynamics model 156 may predict a cell death process for a vulnerable population of cells as the sequence repeat length expands over time.

The client device 104 is shown displaying, via a display device 158, the sequence repeat length distribution 152. By way of example, the display device 158 may display the sequence repeat length distribution 152 as a graph depicting the sequence repeat length (horizontal axis) versus number of sequences (vertical axis). Additionally or alternatively, the display device 158 may display the sequence repeat length distribution 152 as a table of numerical values. It is to be appreciated that the sequencing data 122, the read family alignments 144, the molecule-specific consensus sequences 146, the consensus repeat lengths 148, and/or the sequence repeat length distribution 152 may be also stored in memory, in a single data file or multiple data files, for subsequent access.

In this way, the sequencing data processor 110 generates the sequence repeat length distribution 152 in a manner that neutralizes the distorting effects of the amplification reaction(s), resulting in a more accurate sequence repeat length distribution 152. By way of example, incorporating the molecular labels 126 and using the computational analyses of the repeat length alignment module 136 circumvents amplification reaction bias toward shorter molecules because although shorter molecules may be amplified in higher quantity compared to longer molecules, the molecular labels 126 enable a single consensus sequence to be generated for a nucleic acid molecule of origin regardless of its amplification amount relative to the other nucleic acid molecules of origin. As a result, the sequence repeat length distribution 152 from biological and clinical samples may be used to identify which tissue(s), cell type(s), and/or biological specimen(s) are more affected by a repeat expansion disorder as well as to compare repeat lengths measured at different time points. For instance, doing so may enable the measurement of the extent to which potential treatments have slowed or stopped the expansion of a subject's (e.g., a person, animal, or cell's) DNA repeats.

Having discussed the environment 100, example implementations that employ the environment 100 for single cell/nucleus sequencing or genomic DNA sequencing for sequence repeat length distribution analysis will now be described.

### Sequence Repeat Length Distribution Analysis Example Implementations

Having discussed the environment 100, example implementations that employ the environment 100 for single cell/nucleus sequencing or genomic DNA sequencing for sequence repeat length distribution analysis will now be described.

### Single Cell/Nucleus Sequencing

FIGS. 2A and 2B depict an example workflow 200 in an implementation of preparing single-cell target-sequence sequencing data for repeat length distribution analysis. Where appropriate, reference will be made to components previously introduced in FIG. 1.

Referring first to FIG. 2A, in the example workflow 200, nuclei 202 are extracted from the biological sample 120. By way of example, the nuclei 202 may be prepared from homogenized tissue or another type of cell suspension (e.g., from bodily fluids or cultured cells). The nuclei 202 may be suspended in an aqueous buffer (e.g., water). As a non-limiting example, the aqueous buffer is phosphate-buffered saline (PBS) having bovine serum albumin (BSA) at a desired or appropriate concentration (e.g., 1%). The aqueous buffer may be further supplemented with an RNase inhibitor to reduce an occurrence of RNA degradation, for example.

It is to be appreciated that more than one biological sample 120 may be evaluated in parallel. For instance, the nuclei 202 may be isolated from multiple biological samples, with the samples kept separate from each other throughout the workflow 200 (e.g., in separate tubes, plate wells, or other sample containers).

The nuclei 202 undergo single cell reverse transcription 204 at the nucleic acid amplifier 106. In the example illustrated in FIG. 2A, the single cell reverse transcription 204 incorporates the cell barcodes 128 and the UMIs 130, e.g., via reverse transcription (RT) primers 206. The RT primers 206 are a subset of the primers 124 introduced with respect to FIG. 1, for example. By way of example, the nuclei 202 may be encapsulated in droplets, with each droplet comprising RT primers 206 having a different cell barcode 128 and a plurality of individual UMIs 130. Once encapsulated in the droplets, the nuclei 202 are lysed so that RNA molecules contained therein are primed for reverse transcription using the RT primers 206 having the cell barcodes 128 and the UMIs 130. In at least one implementation, the nucleic acid amplifier 106 is a microfluidic platform, and the RT primers 206 are delivered to respective nuclei 202 during the encapsulation process using beads and microfluidic channels and/or chambers.

Other reagents, such as a reverse transcriptase enzyme, buffer(s), and nucleotides to be incorporated into newly synthesized strands of cDNA (e.g., dNTPs), are also added, resulting in a reverse transcription (RT) reaction mixture 208. In one or more implementations, at least a portion of these reagents are provided in a commercially available kit. The commercially available kit may include a so-called “master mix” of, for example, the reverse transcriptase enzyme, the buffer, the RT primers 206, and/or the nucleotides. Alternatively, however, at least a portion of these reagents may be added separately.

As a non-limiting, illustrative example scenario, the beads are coated with the RT primers 206, with individual beads having RT primers 206 that include a single common cell barcode 128, a plurality of different UMIs 130 (e.g., where no two UMIs 130 are the same on a single bead), and an RNA-targeting oligo (e.g., an oligo dT). As such, a given bead-bound primer of the RT primers 206 may have the following sequence structure:
5′ common adapter-cell barcode-UMI-oligo dT 3′ where “common adapter” denotes a gel-bound portion of the bead-bound primer, “cell barcode” is the cell barcode 128, “UMI” is the UMI 130, and “oligo dT” is the RNA-targeting portion of the bead-bound primer. The common adapter and the oligo dT may be common to all of the bead-bound primer, whereas the cell barcode 128 is specific to an individual bead and the UMI 130 is specific to an individual primer molecule.

The single cell reverse transcription 204 results in barcoded and UMI-labeled cDNA 210. In an example implementation, the single cell reverse transcription 204 results in a library of complementary DNA (cDNA) molecules tagged with the cell barcode 128 and the UMI 130 (e.g., a cDNA library of substantially all of the RNA transcripts of the biological sample 120) as the barcoded and UMI-labeled cDNA 210. By way of example, “whole transcriptome amplification” refers to any amplification method that aims to produce an amplification product that is representative of a population of RNA from the cell from which it was prepared.

An illustrative whole transcriptome amplification (WTA) method facilitates unbiased amplification. In many implementations, WTA is carried out to analyze messenger RNA (mRNA). This is also referred to as “RNA-seq.” The WTA may include reverse transcription to generate first strand cDNA. First strand synthesis may be followed by second strand synthesis. First strand synthesis may include priming of the reverse transcription on a 3′ A-rich sequence of the mRNA, such as on a polyA tail. In example implementation, each mRNA in the biological sample 120 may be reverse transcribed to generate the barcoded and UMI-labeled cDNA 210. The first strand cDNA may have the following sequence structure:
5′ common adapter-cell barcode-UMI-oligo dT-cDNA sequence-TSO adapter 3′ where “common adapter” denotes a gel-bound portion of the barcoded and UMI-labeled cDNA 210 in the RT reaction mixture 208, “cell barcode” is the cell barcode 128, “UMI” is the UMI 130, “oligo dT” is the RNA-targeting portion of RT primers 206, “cDNA sequence” is a sequence that is complementary to a specific mRNA molecule that is reverse transcribed during the single cell reverse transcription 204, and “TSO adapter” is a primer adapter used in a template switching process of the reverse transcription.

The single cell reverse transcription 204 is performed in the nucleic acid amplifier 106. By way of example, the RT reaction mixture 208 is placed in the nucleic acid amplifier 106 in an appropriate volume in an appropriate container (e.g., a tube strip), and the nucleic acid amplifier 106 undergoes a timed series of temperature changes according to a program. In at least one implementation, the nucleic acid amplifier 106 includes a heated lid (e.g., heated to 53° C.) in order to prevent condensation of the RT reaction mixture 208 in tube caps. An illustrative example program is provided in Table 1 below.

TABLE 1

| Step | Temperature (° C.) | Time | |
|---|---|---|---|
| 1 | 53 | 45 | min |
| 2 | 85 | 5 | min |

| | | |
|---|---|---|
| 3 | 4 | ∞ |

The single cell reverse transcription 204 results in the barcoded and UMI-labeled cDNA 210. However, the barcoded and UMI-labeled cDNA 210 is mixed with the reagents of the RT reaction mixture 208 (e.g., the RT primers 206, enzyme, dNTPs, buffer, etc.). Therefore, a first cleanup 212 is performed to isolate the barcoded and UMI-labeled cDNA 210 from the RT reaction mixture 208. Various reaction clean-up techniques may be used, including techniques that enable the amplification products (e.g., the barcoded and UMI-labeled cDNA 210) to be selectively captured. By way of example, the first cleanup 212 may include breaking the beads via temperature changes or chemical treatments to release the barcoded and UMI-labeled cDNA 210 into solution. The first cleanup 212 may further include the use of paramagnetic beads to selectively bind the barcoded and UMI-labeled cDNA 210, e.g., via the common adapter. After washing away other reagents, the selectively bound barcoded and UMI-labeled cDNA 210 may be eluted from the paramagnetic beads, for instance.

The barcoded and UMI-labeled cDNA 210 isolated by the first cleanup 212 is amplified in a transcriptome amplification reaction 214. In the example illustrated in FIG. 2A, a subset of the primers 124 used in the transcriptome amplification reaction 214 is represented as transcriptome primers 216. The transcriptome primers 216 include cDNA primers 218 and optionally include spike-in primers 220. The cDNA primers 218 may be generic cDNA primers that target the 5′ and 3′ sequence adapters of the cDNA molecules, e.g., the common adapter and the TSO adapter. The spike-in primers 220 may be gene-specific primers that are configured to anneal to regions flanking a targeted repeat expansion region. As a non-limiting, illustrative example scenario where the expansion repeat region of HTT is targeted, the spike-in primers 220 may target a region near the 5′ end of HTT. For example, the spike-in primers 220 may have the following sequences:

(SEQ ID NO: 1)
5′-CCCAGAGCCCCATTCATTGCC-3′
(SEQ ID NO: 2)
5′-GGCGACCCTGGAAAAGCTGATG-3′

in order to target the expansion repeat region of HTT. Because the “template switch” step is partially efficient, some of the barcoded and UMI-labeled cDNA 210 may be missing the TSO adapter sequencing, which may result in many first strand cDNAs not being amplified during the transcriptome amplification reaction 214. The addition of spike-in primers increases a yield of the targeted repeat expansion region by making successful amplification independent of the standard, template-agnostic “template switch” step, for instance.

The barcoded and UMI-labeled cDNA 210 and the transcriptome primers 216 are added to additional reagents for the transcriptome amplification reaction 214, resulting in a first amplification reaction mixture 222. The additional reagents may include one or more polymerase enzymes, one or more buffers, nucleotides to be incorporated into newly synthesized strands of DNA (e.g., dNTPs), and water. In one or more implementations, additional additives may be used that help facilitate amplification by modifying the melting (e.g., denaturation) behavior of DNA. In one or more implementations, at least a portion of these reagents are provided in a commercially available kit. The commercially available kit may include a so-called “master mix” of, for example, the polymerase enzyme(s), the buffer, and the nucleotides. Alternatively, however, these reagents may be added separately. A non-limiting example reaction recipe for the first amplification reaction mixture 222 having a 100 μL reaction volume is given below in Table 2.

TABLE 2

| Component | 1X Volume (μL) |
|---|---|
| cDNA | 33 |
| cDNA Primers | 15 |
| Spike-In Primers | 2 |
| Master Mix (buffer, enzyme, dNTPs) | 50 |
| Total | 100 |

The transcriptome amplification reaction 214 is performed in the nucleic acid amplifier 106 to generate amplified barcoded and UMI-labeled cDNA 224. By way of example, the first amplification reaction mixture 222 is placed in the nucleic acid amplifier 106 in an appropriate tube, and the nucleic acid amplifier 106 undergoes a timed series of temperature changes according to a program. In at least one implementation, the nucleic acid amplifier 106 includes a heated lid (e.g., heated to 105° C.) in order to prevent condensation of the first amplification reaction mixture 222 in tube caps. An illustrative example program is provided in Table 3 below.

TABLE 3

| Step | Temperature (° C.) | Time | |
|---|---|---|---|
| 1 | 98 | 3 | min |
| 2 | 98 | 15 | sec |
| 3 | 63 | 20 | sec |
| 4 | 72 | 2 | min |
| 5 | Go to step 2 | 13X | |
| 6 | 72 | 1 | min |

| | | |
|---|---|---|
| 7 | 4 | ∞ |

In the illustrative example shown in Table 3, step 1 is an initial activation step, where the polymerase enzyme is activated, and step 2 is a denaturation step where secondary structures of the barcoded and UMI-labeled cDNA 210 are disrupted. Step 3 is an annealing step where the transcriptome primers 216 bind to targeted regions of the barcoded and UMI-labeled cDNA 210 (e.g., the generic common adapter and the TSO adapter and/or loci upstream and downstream of the HTT expansion repeat region in the example of Huntington's disease). A temperature for step 3 may be adjusted based on an annealing temperature of transcriptome primers 216. Step 4 is an extension step of new strands of cDNA using the polymerase enzyme. The time used during step 4 may be adjusted based on a length of a desired amplification product, as longer products may take more synthesis time than shorter products. Step 5 indicates that steps 2 through 4 may be repeated, e.g., a number of times adjusted based on conditions optimized for targeted cell recovery (e.g., 13 in the present non-limiting example). Step 6 is a final extension step, and step 7 indicates that the reactions may be held indefinitely at a cold temperature for preservation following completion of the programmed cycles.

The transcriptome amplification reaction 214 results in the amplified barcoded and UMI-labeled cDNA 224. However, the amplified barcoded and UMI-labeled cDNA 224 is mixed with the reagents of the first amplification reaction mixture 222 (e.g., the primers 124, enzyme, dNTPs, buffer, etc.). Therefore, a second cleanup 226 is performed to isolate the amplified barcoded and UMI-labeled cDNA 224 from the first amplification reaction mixture 222. Various amplification reaction clean-up techniques may be used, including techniques that enable the amplification products (e.g., the amplified barcoded and UMI-labeled cDNA 224) to be selectively captured over the transcriptome primers 216. By way of example, solid phase reversible immobilization (SPRI) may be used in the second cleanup 226, where paramagnetic beads are used to selectively bind DNA fragments of a selected size range while the transcriptome primers 216, unused nucleotides, enzymes, salts, etc. are washed away. An illustrative example SPRI protocol that may be used as a part of the second cleanup 226 includes the following process:
a. Add an appropriate volume (e.g., 60 μL) of SPRI paramagnetic bead suspension to the amplification product (0.6×) and mix by pipetting.
b. Incubate at room temperature for approximately 5 minutes, or another appropriate length of time that allows the amplified barcoded and UMI-labeled cDNA 224 to bind to the paramagnetic beads.
c. Place the sample on a magnet, with the magnet positioned closer to a cap of the sample tube, to separate the paramagnetic beads from a supernatant of the sample, and then remove the supernatant.
d. Wash the sample by adding an appropriate volume of a washing reagent (e.g., 200 μL of 80% ethanol) to the beads and wait approximately 30 seconds, or another appropriate length of time that allows the washing reagent to wash amplification reaction reagents from the amplified barcoded and UMI-labeled cDNA 224 bound to the paramagnetic beads, and then remove the washing reagent. Repeat for a desired number of washes (e.g., a total of two washes).
e. Centrifuge the sample to spin down the paramagnetic beads and separate out remaining washing reagent, and place the sample on the magnet, with the magnet positioned closer to the bottom of the sample tube. Remove the remaining washing reagent.
f. Add an appropriate volume of an elution reagent (e.g., 40.5 μL of elution buffer) to the paramagnetic beads and mix (e.g., by pipetting) to resuspend the paramagnetic beads.
g. Incubate at room temperature for approximately 2 minutes, or another appropriate length of time that allows the amplified barcoded and UMI-labeled cDNA 224 to elute from the paramagnetic beads.
h. Place the sample tube on the magnet, with the magnet positioned closer to the top of the sample tube, and transfer of the supernatant (e.g., 40 μL) to a new sample tube for a subsequent amplification reaction.

Optionally, a portion of the amplified barcoded and UMI-labeled cDNA 224 may be stored separately as a transcriptome library 228, which may enable a molecular profile of each cell/nucleus to be evaluated, as will be elaborated below. The transcriptome library 228 may also be referred to as a WTA library. The transcriptome library 228 may include “WTA products.”

In at least one implementation, such as in the example implementation of the workflow 200, cDNAs of the targeted repeat expansion region (e.g., from a portion of the amplified barcoded and UMI-labeled cDNA 224 that is not used for the transcriptome library 228) are further amplified via a targeted amplification reaction 230. In the example illustrated in FIG. 2A, the primers 124 used in the targeted amplification reaction 230 include gene-specific primers 232. By way of example, the gene-specific primers 232 may include a small molecule-tagged (e.g., biotinylated) primer designed to anneal to the 5′ end of the targeted repeat expansion region and a 3′ end of one of the spike-in primers 220 and another primer designed to target the common adapter added during the single cell reverse transcription 204. The gene-specific primers 232 facilitate selective amplification of the targeted repeat expansion region. As a non-limiting, illustrative example scenario where the expansion repeat region of HTT is targeted, the gene-specific primers 232 may include the following sequences:

Forward Primer:
(SEQ ID NO: 3)
5′-/5BioagGTGACTGGAGTTCAGACGTGTGCTCTTCCGATCTCCTTC
GAGTCCCTCAAGTCCTTCGTCTCGTGGGCTCGGAGATGTGTATAAGAGA
CAGCCTTCGAGTCCCTCAAGTCCTTC-3′
Reverse Primer:
(SEQ ID NO: 4)
5′-ACACTCTTTCCCTACACGACGCTCTTCCGATCT-3′

where “/5Bfioag” denotes a biotin molecule. The biotin molecule enables the resulting amplicons to be isolated using an affinity agent (e.g., streptavidin beads) in a purification step that will be described below with respect to FIG. 2B.

In the above illustrated example, the gene-specific primers 232 include adapter sequences that may be used to append the indices 132, when used, and sequencing adapters used in sequencing during a subsequent amplification reaction, as will also be described with respect to FIG. 2B. By way of example, the reverse primer of the gene-specific primers 232 may include a first adapter sequence and the forward primer of the gene-specific primers 232 may include a second adapter sequence that is different from the first adapter sequence.

In at least one implementation, a quantitative real-time PCR (qRT-PCR) reaction is used to determine conditions for the targeted amplification reaction 230. By way of example, chimerism may arise when an incomplete amplicon serves as a primer for successive amplification cycles. Chimerism causes the targeted repeat expansion region (e.g., the CAG repeat sequence of HTT) to become associated with the wrong cell barcode 128 and UMI 130, which would produce incorrect sequencing data 122. Chimerism can be particularly problematic when studying repeat expansions, as an incorrect molecule with a short repeat sequence may out-compete (during amplification) longer molecules that are correct but inefficiently amplified. This results in the incorrect molecule becoming the dominant (e.g., most abundant) sequence for that cell barcode 128 and UMI 130. The qRT-PCR enables the number of amplification cycles to be calibrated to the sample so that the targeted amplification reaction 230 may be ended while in log phase, thus preventing or reducing late cycles with incompletely replicated molecules that then act as primers in subsequent amplification cycles. Because the incomplete amplicons tend to be generated when amplification efficiency drops due to limited remaining concentrations of reagents (e.g., the dNTPs, the gene-specific primers 232, and polymerases), chimerism may be prevented or reduced by terminating the targeted amplification reaction 230 while in log phase, e.g., by performing the targeted amplification reaction 230 up to the number of the cycles before the PCR efficiency drops substantially. In general, amplified barcoded and UMI-labeled cDNA 224 with a larger number of founder molecules (e.g. due to more sample input, higher expression of the target gene, or better RNA quality) or more efficient amplification (e.g. due to smaller molecules with fewer sequence repeats in the targeted repeat expansion region) will not need as many amplification cycles during the targeted amplification reaction 230. By way of example, quantification cycle (Cq) values from an amplification curve may be used to judge a number of amplification cycles to perform during the targeted amplification reaction 230 using a pilot reaction of a small aliquot (e.g., a fraction of the amplified barcoded and UMI-labeled cDNA 224 to be amplified in the targeted amplification reaction 230, such as 1/32).

The amplified barcoded and UMI-labeled cDNA 224 and the gene-specific primers 232 are added to additional reagents for the targeted amplification reaction 230, resulting in a second amplification reaction mixture 234. The additional reagents may include one or more polymerase enzymes, one or more buffers, nucleotides to be incorporated into newly synthesized strands of DNA (e.g., dNTPs), and water, similar to that described above for the first amplification reaction mixture 222. A non-limiting example reaction recipe for the first amplification reaction mixture 222 having a 20 μL reaction volume is given below in Table 4.

TABLE 4

| Component | 1X Volume (μL) |
|---|---|
| cDNA | 4 |
| Forward Primer, 10 μM concentration | 1 |
| Reverse Primer, 10 μM concentration | 1 |
| Amplification Additive | 4 |
| Master Mix (buffer, enzyme, dNTPs) | 5 |
| Water | 5 |
| Total | 20 |

The targeted amplification reaction 230 is performed in the nucleic acid amplifier 106 to generate target-enriched barcoded and UMI-labeled cDNA 236. By way of example, the second amplification reaction mixture 234 is placed in the nucleic acid amplifier 106 in an appropriate tube, and the nucleic acid amplifier 106 undergoes a timed series of temperature changes according to a program. In at least one implementation, the nucleic acid amplifier 106 includes a heated lid (e.g., heated to 105° C.) in order to prevent condensation of the second amplification reaction mixture 234 in tube caps. An illustrative example program is provided in Table 5 below.

TABLE 5

| Step | Temperature (° C.) | Time | |
|---|---|---|---|
| 1 | 93 | 3 | min |
| 2 | 93 | 30 | sec |
| 3 | 60 | 30 | sec |
| 4 | 68 | 6 | min |
| 5 | Go to step 2 | 18-22X | |
| 6 | 72 | 10 | min |

| | | |
|---|---|---|
| 7 | 4 | ∞ |

In the illustrative example shown in Table 5, step 1 is an initial activation step, where the polymerase enzyme is activated, and step 2 is a denaturation step where the amplified barcoded and UMI-labeled cDNA 224 is denatured. Step 3 is an annealing step where the gene-specific primers 232 bind to targeted regions of the amplified barcoded and UMI-labeled cDNA 224 (e.g., to capture the HTT expansion repeat region in the example of Huntington's disease). A temperature for step 3 may be adjusted based on an annealing (e.g., melting) temperature of the gene-specific primers 232. Step 4 is an extension step of new strands of cDNA using the polymerase enzyme. The time used during step 4 may be adjusted based on a length of a desired amplification product, as longer products may take more synthesis time than shorter products. Step 5 indicates that steps 2 through 4 may be repeated, e.g., a number of times adjusted based on conditions optimized via the qRT-PCR (e.g., between 18 and 22 times in the present non-limiting example). Step 6 is a final extension step, and step 7 indicates that the reactions may be held indefinitely at a cold temperature for preservation following completion of the programmed cycles.

The targeted amplification reaction 230 results in the target-enriched barcoded and UMI-labeled cDNA 236. In at least one implementation, size separation 238 is performed in order to divide the target-enriched barcoded and UMI-labeled cDNA 236 into two libraries based on molecular size: a short enriched cDNA library 240 and a long enriched cDNA library 242 (see FIG. 2B). The short enriched cDNA library 240 comprises target-enriched barcoded and UMI-labeled cDNA 236 molecules having shorter molecular lengths, and the long enriched cDNA library 242 comprises target-enriched barcoded and UMI-labeled cDNA 236 molecules having longer molecular lengths. It is appreciated that there may be size overlap between the short enriched cDNA library 240 and the long enriched cDNA library 242.

In at least one implementation, the size separation 238 uses a SPRI protocol that is modified from that described above for the second cleanup 226. An illustrative example SPRI protocol that may be used as a part of the size separation 238 includes the following process:
a. Add an appropriate volume of water (e.g., 30 μL) to bring the volume to 50 μL.
b. Add an appropriate volume (e.g., 20 μL) of SPRI paramagnetic bead suspension to the amplification product (0.4×) and mix by pipetting.
c. Incubate at room temperature for approximately 5 minutes, or another appropriate length of time that allows the target-enriched barcoded and UMI-labeled cDNA 236 to bind to the paramagnetic beads.
d. Place the sample on a magnet, with the magnet positioned closer to a cap of the sample tube, to separate the paramagnetic beads from a supernatant of the sample, and then transfer the supernatant to a new tube.
e. Continue processing the bead pellet to generate the long enriched cDNA library 242:
   i. Wash the sample containing the paramagnetic beads by adding an appropriate volume of a washing reagent (e.g., 200 μL of 80% ethanol) to the beads and wait approximately 30 seconds, or another appropriate length of time that allows the washing reagent to wash amplification reaction reagents from the target-enriched barcoded and UMI-labeled cDNA 236 bound to the paramagnetic beads, and then remove the washing reagent. Repeat for a desired number of washes (e.g., a total of two washes).
   ii. Centrifuge the sample to spin down the paramagnetic beads and separate out remaining washing reagent, and place the sample on the magnet, with the magnet positioned closer to the bottom of the sample tube. Remove the remaining washing reagent.
   iii. Add an appropriate volume of an elution reagent (e.g., 11 μL of water, or another low-salt elution buffer) to the paramagnetic beads and mix (e.g., by pipetting) to resuspend the paramagnetic beads.
   iv. Incubate at room temperature for approximately 5 minutes, or another appropriate length of time that allows the target-enriched barcoded and UMI-labeled cDNA 236 to elute from the paramagnetic beads.
   v. Place the sample tube on the magnet, with the magnet positioned closer to the top of the sample tube, and transfer of the supernatant (e.g., 10 μL) to a new sample tube for the long enriched cDNA library 242.
f. Continue processing the transferred supernatant to generate the short enriched cDNA library 240:
   i. Add an appropriate volume (e.g., 20 μL) of SPRI paramagnetic bead suspension to the amplification product (1×) and mix by pipetting.
   ii. Incubate at room temperature for approximately 5 minutes, or another appropriate length of time that allows the target-enriched barcoded and UMI-labeled cDNA 236 to bind to the paramagnetic beads.
   iii. Place the sample on a magnet, with the magnet positioned closer to a cap of the sample tube, to separate the paramagnetic beads from a supernatant of the sample, and then discard the supernatant.
   iv. Wash the sample containing the paramagnetic beads by adding an appropriate volume of a washing reagent (e.g., 200 μL of 80% ethanol) to the beads and wait approximately 30 seconds, or another appropriate length of time that allows the washing reagent to wash amplification reaction reagents from the target-enriched barcoded and UMI-labeled cDNA 236 bound to the paramagnetic beads, and then remove the washing reagent. Repeat for a desired number of washes (e.g., a total of two washes).
   v. Centrifuge the sample to spin down the paramagnetic beads and separate out remaining washing reagent, and place the sample on the magnet, with the magnet positioned closer to the bottom of the sample tube. Remove the remaining washing reagent.
   vi. Add an appropriate volume of an elution reagent (e.g., 11 μL of water, or another low-salt elution buffer) to the paramagnetic beads and mix (e.g., by pipetting) to resuspend the paramagnetic beads.
   vii. Incubate at room temperature for approximately 5 minutes, or another appropriate length of time that allows the target-enriched barcoded and UMI-labeled cDNA 236 to elute from the paramagnetic beads.
   viii. Place the sample tube on the magnet, with the magnet positioned closer to the top of the sample tube, and transfer the supernatant (e.g., 10 μL) to a new sample tube for the short enriched cDNA library 240.

It at least one implementation, target purification 244 is separately performed on the short enriched cDNA library 240 and the long enriched cDNA library 242 in order to generate a short target cDNA library 246 and a long target cDNA library 248, respectively. When the gene-specific primers 232 include a biotinylated primer, the target purification 244 includes purification via streptavidin beads. The streptavidin beads bind the biotin molecule, thus selectively binding the cDNA constructs of the targeted repeat expansion region and enabling other cDNA constructs to be removed. An illustrative example protocol that may be used as a part of the target purification 244 includes the following process:
a. Make an appropriate volume of wash and bind buffer (2X concentration). The wash and bind buffer may be a buffered salt solution, such as tris-buffered saline (TBS) containing a chelating agent (e.g., ethylenediaminetetraacetic acid).
b. Prepare the streptavidin beads:
   i. Resuspend an appropriate volume of the streptavidin beads (e.g., 25 μL for four samples).
   ii. Wash with an appropriate volume (e.g., 1 mL) of 1× wash and bind buffer. Place on a magnet for 1 minute and remove the supernatant.
   iii. Repeat the wash two times for a total of three washes.
   iv. Resuspend the streptavidin beads in 2X wash and bind buffer at twice the original volume (e.g., 50 μL for four reactions)
c. Add the washed and resuspended streptavidin beads to the samples:
   i. Add an appropriate volume (e.g., 10 μL) of the washed and resuspended streptavidin beads to each cDNA sample within sample tubes and mix by pipetting.
   ii. Incubate the sample tubes on a rotator for an appropriate amount of time (e.g., 30 minutes) at room temperature.
   iii. Place the sample tubes on the magnet, with the magnet positioned closer to the bottom of the sample tube.
   iv. Remove the supernatant and place the sample tubes on the magnet, with the magnet positioned closer to the bottom of the sample tube.
   v. Wash with an appropriate volume (e.g., 200 μL) of 1× wash and bind buffer for a total of three washes, with a 1 minute incubation for each wash.
   vi. Spin down the streptavidin beads and remove any extra wash and bind buffer.
d. Resuspend each sample in an appropriate volume of water (e.g., 10 μL) and remove the supernatant from the streptavidin beads.
e. Transfer the supernatant to a new sample tube for each sample.

It is to be appreciated that other purification methods may be used. Moreover, in variations, the size separation 238 may be performed in a different order with respect to the targeted amplification reaction 230 or the target purification 244. By way of example, the size separation 238 may be performed before the targeted amplification reaction 230 or after the target purification 244.

In at least one implementation, the short target cDNA library 246 and the long target cDNA library 248 are further amplified and/or indexed for sequencing via an additional amplification reaction 250. By way of example, the additional amplification reaction 250 uses separate reaction mixtures for the short target cDNA library 246 and the long target cDNA library 248, represented in FIG. 2B as third amplification reaction mixtures 252. In the example illustrated in FIG. 2B, the additional amplification reaction 250 optionally incorporates the indices 132, e.g., via a subset of the primers 124 indicated as amplification primers 254. Single indexing (where one index is incorporated) or dual indexing (where two indices are incorporated) may be used. The dual indexing may be unique dual indexing or combinatorial dual indexing, for example. The indices 132 may be short (e.g., 8-12 nucleotide) sequences that are assigned to a given sample to be sequenced in order to provide an identifying label for the sample for multiplexed sequencing. However, it is to be appreciated that the indices 132 may be omitted, such as when multiplexed sequencing is not used. The amplification primers 254 may be further used to append sequencing adapters 256 that enable flow cell binding during a subsequent sequencing process.

In at least one implementation, the amplification primers 254 target adapter sequences added via the gene-specific primers 232 of the targeted amplification reaction 230 in order to produce an amplified short target cDNA library 258 from the short target cDNA library 246 and an amplified long target cDNA library 260 from the long target cDNA library 248.

A third cleanup 262 may be performed in order to isolate the amplified short target cDNA library 258 and the amplified long target cDNA library 260 from the third amplification reaction mixtures 252. The third cleanup 262 may be performed separately on the amplified short target cDNA library 258 and the amplified long target cDNA library 260. In at least one implementation, the third cleanup 262 may use an SPRI protocol similar to those described above. An illustrative example SPRI protocol that may be used as a part of third cleanup 262 includes the following process:
a. Add an appropriate volume of SPRI paramagnetic bead suspension to the sample (1×) and mix by pipetting. For example, 40 μL of the SPRI paramagnetic bead suspension may be added to 40 μL of the sample.
b. Incubate at room temperature for approximately 5 minutes, or another appropriate length of time that allows the amplified short target cDNA library 258 or the amplified long target cDNA library 260 to bind to the paramagnetic beads.
c. Place the sample on a magnet, with the magnet positioned closer to a cap of the sample tube, to separate the paramagnetic beads from a supernatant of the sample, and then remove the supernatant.
d. Wash the sample by adding an appropriate volume of a washing reagent (e.g., 200 μL of 80% ethanol) to the beads and wait approximately 30 seconds, or another appropriate length of time that allows the washing reagent to wash amplification reaction reagents from the amplified short target cDNA library 258 or the amplified long target cDNA library 260 bound to the paramagnetic beads, and then remove the washing reagent. Repeat for a desired number of washes (e.g., a total of two washes).
e. Centrifuge the sample to spin down the paramagnetic beads and separate out remaining washing reagent, and place the sample on the magnet, with the magnet positioned closer to the bottom of the sample tube. Remove the remaining washing reagent.
f. Add an appropriate volume of an elution reagent (e.g., 11 μL of nuclease-free water or a low-salt buffer) to the paramagnetic beads and mix (e.g., by pipetting) to resuspend the paramagnetic beads.
g. Incubate at room temperature for approximately 5 minutes, or another appropriate length of time that allows the amplified short target cDNA library 258 or the amplified long target cDNA library 260 to elute from the paramagnetic beads.
h. Place the sample tube on the magnet, with the magnet positioned closer to the bottom of the sample tube, and transfer an appropriate volume (e.g., 10 μL) of the supernatant to a new sample tube.

The amplified short target cDNA library 258 and the amplified long target cDNA library 260 are then prepared for sequencing by the DNA sequencer 108. By way of example, the amplified short target cDNA library 258 and the amplified long target cDNA library 260 may be quantified, and an appropriate amount (e.g., 160-500 ng of DNA) used for sequencing. When the indices 132 are incorporated via the additional amplification reaction 250, the amplified short target cDNA library 258 and the amplified long target cDNA library 260 are pooled for sequencing with other index-labeled DNA samples. The other index-labeled DNA samples may be those from another subject (e.g., an individual with the expansion repeat disease of interest or a healthy control), another tissue of a same or different subject, a sample taken at a different time point from the same or different subject, etc., with each sample having a different index sequence or sequences. Moreover, the transcriptome library 228 may be similarly prepared for sequencing by the DNA sequencer 108.

In at least one implementation, the resulting sequencing data 122 includes barcoded and UMI-labeled target cDNA library reads 264 and barcoded and UMI-labeled transcriptome library reads 266. The barcoded and UMI-labeled target cDNA library reads 264 comprise the sequencing data 122 corresponding to the amplified short target cDNA library 258 and the amplified long target cDNA library 260, while the barcoded and UMI-labeled transcriptome library reads 266 comprise the sequencing data 122 corresponding to the transcriptome library 228. By way of example, the cell barcodes 128 enable genome-wide RNA expression to be correlated to respective consensus repeat lengths 148 for the targeted repeat expansion region, thus making it possible to appreciate the relationship between the consensus repeat lengths 148 and potentially morbid gene expression changes.

FIG. 3 depicts an illustrative example process 300 for synthesizing cell barcoded and UMI-labeled cDNA from RNA for single cell/nucleus sequencing for sequence repeat length distribution analysis. The process 300, for instance, highlights one implementation of the single cell reverse transcription 204 of FIG. 2A. As such, where appropriate, reference will be made to components previously described with reference to FIGS. 1-2B. It is to be appreciated that the process 300 is a simplified example, and the relative lengths of the various sequence portions are not to scale. Moreover, for illustrative clarity, particular sequence portions are not labeled in every portion of the figure.

The process 300 includes a primer annealing step 302, a reverse transcription step 304, a template switching oligo priming step 306, and a template extension step 308. The primer annealing step 302 depicts a first RNA molecule 310, which may be a molecule of mRNA from a first cell of the biological sample 120, and a second RNA molecule 312, which may be a molecule of mRNA from a second cell of the biological sample 120. The first RNA molecule 310 includes a first RNA sequence (e.g., “RNA sequence 1,” dark shading) at the 5′ end and an A-rich sequence (such as a polyA tail) at the 3′ end. Similarly, the second RNA molecule 312 includes a second RNA sequence (e.g., “RNA sequence 2,” dark shading) at the 5′ end and the A-rich sequence at the 3′ end. In the present example, the first RNA molecule 310 includes a repeat expansion region 314 (e.g., depicted by diagonal shading) having a first length 316, and the second RNA molecule 312 includes a repeat expansion region 314 having a second length 318. In the present example, the second length 318 is longer than the first length 316. That is, more sequence repeats are included in the repeat expansion region 314 having the second length 318 than in the repeat expansion region 314 having the first length 316.

The first RNA molecule 310 is encapsulated in a first droplet 320 along with a first bead 322 (e.g., “bead 1”). The first bead 322 includes a plurality of primers positioned on its surface, including a first primer 324. The first primer 324 includes, from 5′ to 3′, an adapter sequence (e.g., “adapter”), a first barcode (e.g., “barcode 1”), a first UMI (e.g., “UMI1”), and an oligo dT (e.g., “dT”). The first primer 324 is attached to the surface of the first bead 322 at the adapter sequence (e.g., on the 5′ end). It is appreciated that other primers on the surface of the first bead 322 may include the first barcode and UMIs 130 having different sequences than the first UMI.

The second RNA molecule 312 is encapsulated in a second droplet 326 along with a second bead 328 (e.g., “bead 2”). The second bead 328 includes a plurality of primers positioned on its surface, including a second primer 330. The second primer 330 includes, from 5′ to 3′, an adapter sequence (e.g., “adapter”), a second barcode (e.g., “barcode 2”), a second UMI (e.g., “UMI2”), and the oligo dT (e.g., “dT”). The second primer 330 is attached to the surface of the second bead 328 at the adapter sequence (e.g., on the 5′ end). It is appreciated that other primers on the surface of the second bead 328 may include the second barcode and UMIs 130 having different sequences than the second UMI.

During the primer annealing step 302, the first primer 324 anneals to the A-rich sequence of the first RNA molecule 310 via complementary base pairing between the A-rich sequence of the first RNA molecule 310 and the oligo dT of the first primer 324. Similarly, the second primer 330 anneals to the A-rich sequence of the second RNA molecule 312 via complementary base pairing between the A-rich sequence of the second RNA molecule 312 and the oligo dT of the second primer 330. Because the first RNA molecule 310 is encapsulated in the first droplet 320, the first RNA molecule 310 is isolated from the second bead 328 and the second primer 330. Thus, the first RNA molecule 310, and any other RNA molecules of the first cell, may not bind to the second primer 330. As such, RNA molecules from the first cell, including the first RNA molecule 310, are labeled with the first barcode via the process 300. Similarly, because the second RNA molecule 312 is encapsulated by the second droplet 326, the second RNA molecule 312 is isolated from the first bead 322 and the first primer 324. Thus, the second RNA molecule 312, and any other RNA molecules of the second cell, may not bind to the first primer 324. As such, RNA molecules from the second cell, including the second RNA molecule 312, are labeled with the second barcode via the process 300.

For illustrative clarity, the first droplet 320 and the second droplet 326 are not indicated in the reverse transcription step 304, the oligo priming step 306, and the template extension step 308. However, it is to be appreciated that the corresponding components remain encapsulated in the respective droplets throughout the process 300.

During the reverse transcription step 304 in the first droplet 320, a reverse transcriptase enzyme (not shown) extends the first primer 324 in the 3′ direction by adding nucleotides that are complementary to the first RNA molecule 310, thus producing a complement to the first RNA molecule 310 as a first cDNA sequence 332 (e.g., “cDNA sequence 1). By way of example, the reverse transcription step 304 results in nucleotides complementary to the first RNA molecule 310 extending from the first primer 324. As such, the first cDNA sequence 332 includes a complement of the repeat expansion region 314 having the first length 316. When the reverse transcriptase enzyme reaches the 5′ end of the first RNA molecule 310, terminal transferase activity adds a sequence of non-templated nucleotides (e.g., a sequence of nucleotides that is not included in the first RNA molecule 310) to the 3′ end of the first cDNA sequence 332. In the example depicted in FIG. 3, the non-templated nucleotides comprise a motif of C nucleotides.

Similarly, during the reverse transcription step 304 in the second droplet 326, the reverse transcriptase enzyme (not shown) extends the second primer 330 in the 3′ direction by adding nucleotides that are complementary to the second RNA molecule 312, thus producing a complement to the second RNA molecule 312 as a second cDNA sequence 334 (e.g., “cDNA sequence 2). By way of example, the reverse transcription step 304 results in nucleotides complementary to the second RNA molecule 312 extending from the second primer 330. As such, the second cDNA sequence 334 includes a complement of the repeat expansion region 314 having the second length 318. When the reverse transcriptase enzyme reaches the 5′ end of the second RNA molecule 312, terminal transferase activity adds a sequence of non-templated nucleotides (e.g., a sequence of nucleotides that is not included in the second RNA molecule 312) to the 3′ end of the second cDNA sequence 334, such as mentioned above.

The non-templated nucleotides provide a motif for annealing by a template switching oligo (TSO) primer 336 during the template switching oligo priming step 306. The TSO primer 336 includes a complementary sequence to the non-templated nucleotides at the 3′ end and a TSO sequence at the 5′ end.

During the template extension step 308, the reverse transcriptase enzyme switches to using the TSO primer 336 as a template for extending the cDNA (e.g., the first cDNA sequence 332 or the second cDNA sequence 334) beyond the 5′ end of the RNA sequence (e.g., the first RNA sequence for the first RNA molecule 310 or the second RNA sequence of the of the second RNA molecule 312, respectively). The reverse transcriptase enzyme extends the cDNA by synthesizing a complementary portion of the TSO primer 336, resulting in each cDNA molecule being appended with a TSO adapter sequence 338.

The template extension step 308 results in a first cDNA construct 340 including the first cDNA sequence 332 and a second DNA construct 342 including the second cDNA sequence 334. The first cDNA construct 340 is an amplicon of the first RNA molecule 310 and has a 5′ to 3′ structure of the adapter sequence, the first barcode, the first UMI, the oligo dT, the first cDNA sequence 332 (including the repeat expansion region 314 having the first length 316), the non-templated nucleotides, and the TSO adapter sequence 338. The second DNA construct 342 is an amplicon of the second RNA molecule 312 and has a 5′ to 3′ structure of the adapter sequence, the second barcode, the second UMI, the oligo dT, the second cDNA sequence 334 (including the repeat expansion region 314 having the second length 318), the non-templated nucleotides, and the TSO adapter sequence 338. In at least one implementation, second strand synthesis is performed to generate double-stranded cDNA of each cDNA construct, which may be further amplified in WTA (e.g., the transcriptome amplification reaction 214) and/or targeted amplification procedures (e.g., the targeted amplification reaction 230), such as those described above with respect to FIGS. 2A and 2B. By way of example, the transcriptome primers 216 used in the transcriptome amplification reaction 214 may anneal to the adapter sequence and the TSO adapter, thus amplifying the cDNA constructs regardless of the cDNA sequence therein.

It is to be appreciated that the process 300 is one example process that may be used to incorporate the molecular labels 126 in a single cell/nucleus sequencing implementation and that other processes may be used. By way of example, the cells or extracted nuclei may be isolated from each other and labeled in a cell/nuclei-specific manner using other techniques without departing from the spirit or scope of the present disclosure.

### Genomic DNA Sequencing

FIG. 4 depicts an example workflow 400 in an implementation of preparing a genomic DNA sample for sequence repeat length distribution analysis. Where appropriate, reference will be made to components previously introduced in FIG. 1.

In the example workflow 400, DNA isolation 402 is performed on the biological sample 120 to extract genomic DNA 404. By way of example, the biological sample 120 may include whole cells and/or cell debris (e.g., lysed cells) derived from a tissue or bodily fluid (e.g., blood, cerebrospinal spinal fluid, or the like). As a non-limiting, illustrative example, the biological sample 120 includes cell lysate derived from brain tissue. Example techniques that may be used for the DNA isolation 402 include spin column purification (where DNA is selectively bound to a matrix of a column within a centrifuge tube, enabling contaminants to be washed from the column and centrifuged away prior to eluting the DNA from the matrix) and phenol-chloroform extraction (where phenol and chloroform are used to separate DNA from other cellular components), although other DNA extraction techniques may be used.

It is to be appreciated that more than one biological sample 120 may be evaluated in parallel. For instance, multiple biological samples may undergo the DNA isolation 402 and subsequent amplification reactions, with the samples kept separate from each other throughout the workflow 400 (e.g., in separate tubes, plate wells, or other sample containers).

The genomic DNA 404 undergoes a first amplification reaction 406 at the nucleic acid amplifier 106. In the example illustrated in FIG. 4, the first amplification reaction 406 incorporates the UMIs 130, e.g., via the primers 124. As a non-limiting, illustrative example scenario where the expansion repeat region of HTT is targeted, an example forward primer sequence of the primers 124 used in the first amplification reaction 406 is:

(SEQ ID NO: 5)
5′ TCGTCGGCAGCGTCAGATGTGTATAAGAGACAG(N)(N)(N)(N)
N)(N)(N)(N)(N)(N)(N)(N)GGCTGAGGAAGCTGAGGAG 3′

where (N) denotes a random nucleotide. In this example, the UMIs 130 of the forward primers include a twelve nucleotide random sequence region between a 5′ constant region and a 3′ constant region that are the same for the forward primers. In at least one implementation, the nucleotides in the random sequence region have an approximately equivalent mixture such that A, C, G, and T are present at a given (N) position in approximately 25% of the forward primers (typically denoted as N:25252525). However, other mixed base ratios may be used, such as N:20202040 denoting a 20% A, 20% C, 20% G, 40% T mixture.

The 3′ constant region of the forward primers targets a genetic locus that is upstream of a targeted expansion repeat region (e.g., toward the 5′ end of the sense strand relative to the targeted expansion repeat region), while the 5′ constant region includes a site targeted by primers used in a subsequent amplification reaction, as will be elaborated below. Together, the 5′ constant region and the UMI 130 result in a 5′ forward overhang with respect to the targeted genetic locus (e.g., the HTT expansion repeat region). In the present example of the forward primer sequence, the 3′ constant region targets a position upstream of the HTT expansion repeat region, such as through complementary base pairing with the antisense strand of DNA.

Continuing with the above illustrative example scenario, an example reverse primer sequence of the primers 124 used in the first amplification reaction 406 is:

(SEQ ID NO: 6)
5′ GTCTCGTGGGCTCGGAGATGTGTATAAGAGACG(N)(N)(N)
(N)(N)(N)(N)(N)(N)(N)(N)(N)CCTTCGAGTCCCTCAAGT
CCTTC 3′

where (N) again denotes a random nucleotide. Similar to the forward primers, in this example, the UMIs 130 of the reverse primers include a twelve nucleotide random sequence region between a 5′ constant region and a 3′ constant region that are the same for the reverse primers. The nucleotides in the random sequence region of the reverse primers may have a same mixture as the forward primers or a different mixture than the forward primers.

The 3′ constant region of the reverse primers targets a genetic locus that is downstream of the targeted expansion repeat region (e.g., toward the 3′ end of the sense strand relative to the targeted expansion repeat region), while the 5′ constant region includes a site targeted by primers used in the subsequent amplification reaction. For instance, the 5′ constant region and the UMI 130 result in a 5′ reverse overhang with respect to the targeted genetic locus. In the present example of the reverse primer sequence, the 3′ constant region targets a position downstream of the HTT expansion repeat region (e.g., through complementary base pairing with the sense strand of DNA).

As such, the primers 124 used in the first amplification reaction 406 include a mixture of forward primers and a mixture of reverse primers, with individual forward and reverse primers having different random nucleotide sequences for the UMIs 130 with respect to the other forward and reverse primers, respectively.

The genomic DNA 404 and the primers 124 having the UMIs 130 are added to additional reagents for the first amplification reaction 406, resulting in a first amplification reaction mixture 408. The additional reagents may include one or more polymerase enzymes, one or more buffers, nucleotides to be incorporated into newly synthesized strands of DNA (e.g., dNTPs), and water. In one or more implementations, additional additives may be used that help facilitate amplification by modifying the melting (e.g., denaturation) behavior of DNA. In one or more implementations, at least a portion of these reagents are provided in a commercially available kit. The commercially available kit may include a so-called “master mix” of, for example, the polymerase enzyme(s), the buffer, and the nucleotides. Alternatively, however, these reagents may be added separately. A non-limiting example reaction recipe for the first amplification reaction mixture 408 having a 20 μL reaction volume is given below in Table 6.

TABLE 6

| Component | 1X Volume (μL) |
|---|---|
| Genomic DNA | Varies based on |
| | concentration |
| Forward Primers, 10 μM concentration | 1 |
| Reverse Primers, 10 μM concentration | 1 |
| Amplification Additive | 4 |
| Master Mix (buffer, enzyme, dNTPs) | 5 |
| Water | to 20 |
| Total | 20 |

The first amplification reaction 406 is performed in the nucleic acid amplifier 106, e.g., the thermal cycler, in a manner that facilitates incorporation of a single set of UMIs 130 for respective DNA molecules in the genomic DNA 404. By way of example, the first amplification reaction mixture 408 is placed in the nucleic acid amplifier 106 in an appropriate tube, and the nucleic acid amplifier 106 undergoes a timed series of temperature changes according to a program. In at least one implementation, the nucleic acid amplifier 106 includes a heated lid (e.g., heated to 105° C.) in order to prevent condensation of the first amplification reaction mixture 408 in tube caps. An illustrative example program is provided in Table 7 below.

TABLE 7

| Step | Temperature (° C.) | Time | |
|---|---|---|---|
| 1 | 93 | 3 | min |
| 2 | 93 | 30 | sec |
| 3 | 60 | 30 | sec |
| 4 | 68 | 6 | min |
| 5 | Go to step 2 | 1-3X | |
| 6 | 72 | 10 | min |

| | | |
|---|---|---|
| 7 | 4 | ∞ |

In the illustrative example shown in Table 7, step 1 is an initial activation step, where the polymerase enzyme is activated, and step 2 is a denaturation step where the double-stranded genomic DNA 404 is separated into single strands. Step 3 is an annealing step where the primers 124 having the UMIs 130 bind to targeted regions of the genomic DNA 404 (e.g., loci upstream and downstream of the HTT expansion repeat region in the example of Huntington's disease). A temperature for step 3 may be adjusted based on an annealing (e.g., melting) temperature of the primers 124. Step 4 is an extension step of new, complementary strands of a targeted portion of the DNA using the polymerase enzyme. The time used during step 4 may be adjusted based on a length of a desired amplification product, as longer products may take more synthesis time than shorter products. Step 5 indicates that steps 2 through 4 may be repeated, e.g., between one and three times depending on conditions optimized for a target of interest. Step 6 is a final extension step, and step 7 indicates that the reactions may be held indefinitely at a cold temperature for preservation following completion of the programmed cycles.

It is to be appreciated that according to the example provided in Table 7, two to four reaction cycles are used in order to incorporate a single pair of UMIs 130 (e.g., one forward primer UMI and one reverse primer UMI) into a DNA molecule of origin. For example, the first amplification reaction 406 may include between one and five cycles. In at least one implementation, the number of reaction cycles used in the first amplification reaction 406 may be adjusted based on an amount of the genomic DNA 404. For instance, the number of reaction cycles used in the first amplification reaction 406 may be decreased when the amount of the genomic DNA 404 is higher and increased when the amount of the genomic DNA 404 is lower. The number of reaction cycles used for the first amplification reaction 406 may be selected to reduce an incidence of re-priming, which may replace a UMI 130 that has been incorporated during a previous reaction cycle with a new UMI 130, for instance. However, the analysis of the resulting sequencing data 122 by the repeat length alignment module 136 may enable such re-priming events to be identified and the corresponding reads grouped in the read families 140 based on sharing a single matching (e.g., as matched through fuzzy matching) UMI sequence.

The first amplification reaction 406 results in UMI-labeled DNA 410. However, the UMI-labeled DNA 410 is mixed with the reagents of the first amplification reaction mixture 408 (e.g., the primers, enzyme, dNTPs, buffer, etc.). Therefore, a first cleanup 412 is performed to isolate the UMI-labeled DNA 410 from the first amplification reaction mixture 408. Various amplification reaction clean-up techniques may be used, including techniques that enable the amplification products (e.g., the UMI-labeled DNA 410) to be selectively captured over genomic DNA and the primers 124. By way of example, solid phase reversible immobilization (SPRI) may be used in the first cleanup 412, where paramagnetic beads are used to selectively bind DNA fragments of a selected size range while genomic DNA, unused nucleotides, enzymes, salts, etc. are washed away. An illustrative example SPRI protocol that may be used as a part of the first cleanup 412 includes the following process:
a. Bring the volume of the first amplification reaction mixture 408 up to 50 μL with nuclease-free water in a sample tube.
b. Add 90 μL of SPRI paramagnetic bead suspension to 50 μL product (1.8×) and mix by pipetting.
c. Incubate at room temperature for approximately 5 minutes, or another appropriate length of time that allows the UMI-labeled DNA 410 to bind to the paramagnetic beads.
d. Place the sample on a magnet, with the magnet positioned closer to a cap of the sample tube, to separate the paramagnetic beads from a supernatant of the sample, and then remove the supernatant.
e. Wash the sample by adding an appropriate volume of a washing reagent (e.g., 200 μL of 80% ethanol) to the beads and wait approximately 30 seconds, or another appropriate length of time that allows the washing reagent to wash amplification reaction reagents from the UMI-labeled DNA 410 bound to the paramagnetic beads, and then remove the washing reagent. Repeat for a desired number of washes (e.g., a total of two washes).
f. Centrifuge the sample to spin down the paramagnetic beads and separate out remaining washing reagent, and place the sample on the magnet, with the magnet positioned closer to the bottom of the sample tube. Remove the remaining washing reagent.
g. Add an appropriate volume of an elution reagent (e.g., 11 μL of nuclease-free water or a low-salt buffer) to the paramagnetic beads and mix (e.g., by pipetting) to resuspend the paramagnetic beads.
h. Incubate at room temperature for approximately 5 minutes, or another appropriate length of time that allows the UMI-labeled DNA 410 to elute from the paramagnetic beads.
i. Place the sample tube on the magnet, with the magnet positioned closer to the bottom of the sample tube, and transfer 10 μL of the supernatant to a new sample tube for a subsequent amplification reaction.

The UMI-labeled DNA 410 isolated by the first cleanup 412 is further amplified in a second amplification reaction 414. In the example illustrated in FIG. 4, the second amplification reaction 414 optionally incorporates the indices 132, e.g., via the primers 124. Single indexing (where one index is incorporated) or dual indexing (where two indices are incorporated) may be used. The dual indexing may be unique dual indexing or combinatorial dual indexing, for example. However, it is to be appreciated that the indices 132 may be omitted, such as when multiplexed sequencing is not used.

An illustrative example of the primers 124 used in the second amplification reaction 414 will now be described. Continuing with the non-limiting, illustrative example scenario introduced above, an example sequence of a first amplification primer used for the second amplification reaction 414 is:

(SEQ ID NO: 7)
5′ AATGATACGGCGACCACCGAGATCTACAC[first index]TCGT
CGGCAGCGTC 3′

where [first index] denotes a first index sequence. In this example, the 3′ region following the first index sequence targets at least a portion of the 5′ region of the forward primer used in the first amplification reaction 406. Thus, the first amplification primer is configured to anneal to the UMI-labeled DNA 410 and incorporate the first index sequence during the second amplification reaction 414. The first index sequence is selected from a plurality of known index sequences and is a short (e.g., 8-12 nucleotide) sequence that is assigned to a given sample to be sequenced. The 5′ region preceding the first index may be a first sequencing adapter (e.g., a first flow cell binding sequence) configured to attach to a flow cell surface for sequencing (e.g., the 5′ end of a flow cell oligonucleotide).

Continuing with the non-limiting, illustrative example scenario introduced above, an example sequence for a second amplification primer of the primers 124 for the second amplification reaction 414 is:

(SEQ ID NO: 8)
5′ CAAGCAGAAGACGGCATACGAGAT[second index]GTCTCG
TGGGCTCGG 3′

where [second index] denotes a second index sequence. In this example, the 3′ region following the second index sequence targets at least a portion of the 5′ region of the reverse primer used in the first amplification reaction 406. Thus, the second amplification primer is configured to anneal to the UMI-labeled DNA 410 and incorporate the second index sequence during the second amplification reaction 414. The second index sequence is selected from a plurality of known index sequences. Similar to the first index sequence, the second index sequence is a short (e.g., 8-12 nucleotide) sequence that is assigned to the given sample to be sequenced. The 5′ region preceding the second index may be a second sequencing adapter (e.g., a second flow cell binding sequence) configured to attach to a flow cell surface for sequencing (e.g., the 3′ end of a flow cell oligonucleotide).

Unlike the UMIs 130, where each forward and reverse primer molecule includes a different random UMI sequence, one forward amplification primer molecule having a known first index sequence and one reverse amplification molecule having a known second index sequence can be used per sample in order to provide identifying labels to the sample. This allows one sample to be distinguished from another in a multiplexed sequencing reaction. By way of example, the indices 132 provide additional molecular labels (e.g., tags) so that multiple samples may be pooled for sequencing, thus reducing resource costs and increasing sequencing bandwidth.

The UMI-labeled DNA 410 and the amplification primers optionally having the indices 132 are added to additional reagents for the second amplification reaction 414 in a manner similar to that described above for the first amplification reaction 406, resulting in a second amplification reaction mixture 416. A non-limiting example reaction recipe for the second amplification reaction mixture 416 having a 40 μL reaction volume is given below in Table 8.

TABLE 8

| Component | 1X Volume (μL) |
|---|---|
| UMI-Labeled DNA | 10 |
| First Amplification Primer, 10 μM | 2 |
| concentration | |
| Second Amplification Primer, 10 μM | 2 |
| concentration | |
| Amplification Additive | 8 |
| Master Mix (buffer, enzyme, dNTPs) | 10 |
| Water | 8 |
| Total | 40 |

The second amplification reaction 414 is performed in the nucleic acid amplifier 106 in a manner that amplifies the UMI-labeled DNA 410 and optionally introduces the indices 132 to the UMI-labeled DNA 410. By way of example, the second amplification reaction mixture 416 is placed in the nucleic acid amplifier 106 in an appropriate tube. The nucleic acid amplifier 106 undergoes a timed series of temperature changes according to a program that is different than the program used during the first amplification reaction 406. An illustrative example program is provided in Table 9 below, which may be used with a heated lid, as before.

TABLE 9

| Step | Temperature (° C.) | Time | |
|---|---|---|---|
| 1 | 93 | 3 | min |
| 2 | 93 | 30 | sec |
| 3 | 60 | 30 | sec |
| 4 | 68 | 6 | min |
| 5 | Go to step 2 | 23-29X | |
| 6 | 72 | 10 | min |

| | | |
|---|---|---|
| 7 | 4 | ∞ |

In the illustrative example shown in Table 9, step 1 is an initial activation step, where the polymerase enzyme is activated, and step 2 is a denaturation step where the UMI-labeled DNA 410 is separated into single strands. Step 3 is an annealing step where the primers 124 having the indices 132 bind to the target overhang regions of the UMI-labeled DNA 410, and step 4 is an extension step of new, complementary strands of DNA using the one or more polymerase enzymes. As in the first amplification reaction 406, the time used during step 4 of the second amplification reaction 414 may be adjusted based on a length of a desired amplification product, as longer products may take more synthesis time than shorter products. Step 5 indicates that steps 2 through 4 may be repeated, e.g., between 23 and 29 times depending on conditions optimized for a target of interest. By way of example, the second amplification reaction 414 may include between 20 and 30 reaction cycles. Step 6 is a final extension step, and step 7 indicates that the reaction may be held indefinitely at a cold temperature for preservation following completion of the programmed cycles.

In at least one implementation, the number of reaction cycles performed during the second amplification reaction 414 is greater than the number of reaction cycles performed in the first amplification reaction 406 in order to generate enough product for quantification and subsequent sequencing. As a non-limiting example, the number of reaction cycles performed during the second amplification reaction 414 is in a range between six and forty cycles. Moreover, the number of reaction cycles performed during the second amplification reaction 414 may be adjusted based on the amount of the UMI-labeled DNA 410, the number of reaction cycles performed during the first amplification reaction 406, and/or the sequencing technology to be used. By way of example, the number of reaction cycles performed during the second amplification reaction 414 may be increased when the amount of the UMI-labeled DNA 410 is lower, the number of reaction cycles performed during the first amplification reaction 406 is lower, and/or the sequencing technology uses a larger amount of DNA. Conversely, the number of reaction cycles performed during the second amplification reaction 414 may be decreased when the amount of the UMI-labeled DNA 410 is higher, the number of reaction cycles performed during the first amplification reaction 406 is higher, and/or the sequencing technique uses a smaller amount of DNA.

The second amplification reaction 414 results in amplified UMI-labeled DNA 418, which is optionally indexed through incorporation of the indices 132. Similar to the first amplification reaction 406, the amplified UMI-labeled DNA 418 is mixed with the reagents of the second amplification reaction mixture 416 (e.g., the primers, enzymes, dNTPs, buffers, etc.). Therefore, a second cleanup 420 is performed to isolate the amplified UMI-labeled DNA 418 from the second amplification reaction mixture 416. A technique used for the second cleanup 420 may be the same or different than that used for the first cleanup 412. By way of example, SPRI cleanup may be used, such as according to the example SPRI protocol outlined above. Additionally, or alternatively, spin-column purification may be used. In at least one implementation, multiple cleanup techniques may be combined. By way of example, SPRI cleanup may be followed by gel electrophoresis. An illustrative example gel electrophoresis protocol that may be used as a part of the second cleanup 420 includes the following process:
a. Prepare an appropriate percentage agarose gel for the amplified UMI-labeled DNA 418 amplicon size (e.g., 2%) in 1X buffer (e.g., Tris base, acetic acid and EDTA, or TAE, buffer) with a gel stain for ultraviolet (UV) light-mediated visualization of DNA bands. Cast the agarose gel with an appropriate number of sample wells and/or an appropriate well volume.
b. Once the agarose gel is set, orient the agarose gel in an electrophoresis chamber so that the amplified UMI-labeled DNA 418 will migrate through the agarose gel from sample wells toward a positive electrode.
c. Fill the electrophoresis chamber with the 1× buffer.
d. Load the amplified UMI-labeled DNA 418 into one or more sample wells in the agarose gel using loading dye. Load an additional sample well with an appropriate molecular weight ladder for the amplicon size of the amplified UMI-labeled DNA 418.
e. Run the agarose gel at an appropriate voltage (e.g., 130 V) for an appropriate length of time (e.g., approximately 40 minutes) or until distinct bands appear under UV light.
f. Excise the appropriate product bands (e.g., bands having a molecular weight consistent with the amplicon size, as judged based on the molecular weight ladder).
g. Extract the amplified UMI-labeled DNA 418 from the excised gel, such as by gel lysis and spin column purification.

The amplified UMI-labeled DNA 418 is then prepared for sequencing by the DNA sequencer 108. By way of example, the amplified UMI-labeled DNA 418 may be quantified, and an appropriate amount (e.g., 160-500 ng of DNA) used for sequencing. When the indices 132 are incorporated via the second amplification reaction 414, the amplified UMI-labeled DNA 418 is pooled for sequencing with other UMI and index-labeled DNA samples. The other UMI and index-labeled DNA samples may be those from another subject (e.g., an individual with the expansion repeat disease of interest or a healthy control), another tissue of a same or different subject, a sample taken at a different time point from the same or different subject, etc., with each sample having different first and second index sequences.

FIG. 5 depicts an illustrative example amplification reaction 500 for introducing unique molecular identifiers (UMIs) for labeling individual DNA molecules in a bulk sample. The illustrative example amplification reaction 500, for instance, is one implementation of the first amplification reaction 406 described above with respect to FIG. 4. As such, where appropriate, reference will be made to components previously described with reference to FIG. 4. It is to be appreciated that the illustrative example amplification reaction 500 is a simplified example, and the relative lengths of the various sequence portions are not to scale.

The illustrative example amplification reaction 500 depicts the genomic DNA 404 as including a first DNA molecule 502 and a second DNA molecule 504. It is to be appreciated that the genomic DNA 404 may include a vast quantity of DNA molecules, and two DNA molecules are shown for illustrative clarity. The first DNA molecule 502 includes a repeat expansion region 506 (e.g., depicted by diagonal shading) having a first sequence repeat length 508, while the second DNA molecule 504 includes a second sequence repeat length 510 for the repeat expansion region 506. In the present example, the second sequence repeat length 510 is longer than the first sequence repeat length 508. That is, more sequence repeats are included in the repeat expansion region 506 having the second sequence repeat length 510 than having the first sequence repeat length 508.

The first DNA molecule 502 and the second DNA molecule 504 are depicted as double-stranded molecules having a sense strand (depicted by darker shading) and an antisense strand (depicted by lighter shading). Denaturation causes the sense and anti-sense strands to separate. By way of example, the first DNA molecule 502 separates into a first antisense strand 512 and a first sense strand 514, and the second DNA molecule 504 separates into a second antisense strand 516 and a second sense strand 518.

During primer annealing 520, a first forward primer 522 anneals to the first antisense strand 512, and a first reverse primer 524 anneals to the first sense strand 514. Similarly, a second forward primer 526 anneals to the second antisense strand 516, and a second reverse primer 528 anneals to the second sense strand 518. The first forward primer 522 includes, from 5′ to 3′, a first tag (e.g., “tag 1”), a first UMI (e.g., “UMI1”), and a first locus-specific sequence (e.g., “LSS1”). The second forward primer 526 includes, from 5′ to 3′, the first tag, a second UMI (e.g., “UMI2”), and the first locus-specific sequence. The first and second UMIs include a defined number of nucleotides (e.g., between eight and twelve nucleotides) and have different sequences with respect to each other and with respect to the UMIs of other forward primers included in the amplification reaction that are not specifically shown.

In contrast to the first UMI and the second UMI, the first tag and the first locus-specific sequence are common to the first forward primer 522 and the second forward primer 526 as well as other forward primers used in the amplification reaction that are not specifically shown. The first tag provides a forward amplification primer binding location for a subsequent amplification reaction. The first locus-specific sequence selectively targets and binds (e.g., anneals) to a region upstream of the repeat expansion region 506, e.g., on the anti-sense strand of a given DNA molecule. Thus, the first forward primer 522 and the second forward primer 526 are the same except for the sequences of their respective UMIs.

The first reverse primer 524 includes, from 5′ to 3′, a second tag (e.g., “tag 2”) a third UMI (e.g., “UMI3”), and a second locus-specific sequence (e.g., “LSS2”). The second reverse primer 528 includes, from 5′ to 3′, the second tag, a fourth UMI (e.g., “UMI4”), and the second locus-specific sequence. The third and fourth UMIs include a defined number of nucleotides (e.g., between eight and twelve nucleotides) and have different sequences with respect to each other and with respect to the UMIs of other reverse primers included in the amplification reaction that are not specifically shown.

Similar to the first tag and the first locus-specific sequence described above for the forward primers, the second tag and the second locus-specific sequence are common to the first reverse primer 524 and the second reverse primer 528 as well as other reverse primers used in the amplification reaction that are not specifically shown. The second tag provides a reverse amplification primer binding location for a subsequent amplification reaction. The second locus-specific sequence selectively targets and binds (e.g., anneals) to a region downstream of the repeat expansion region 506, e.g., on the sense strand of a given DNA molecule. Thus, the first reverse primer 524 and the second reverse primer 528 are the same except for the sequences of their respective UMIs.

During extension 530, a polymerase enzyme (not shown) extends the primers in the 3′ direction by adding nucleotides that are complementary to a corresponding strand of DNA to synthesize new complementary strands of DNA. By way of example, the extension 530 process results in nucleotides complementary to the first antisense strand 512 extending from the first forward primer 522, thus copying the repeat expansion region 506 of the first sense strand 514. Similarly, the extension 530 process results in nucleotides complementary to the first sense strand 514 extending from the first reverse primer 524, thus copying the repeat expansion region 506 of the first antisense strand 512. The second forward primer 526 and the second reverse primer 528 are extended in a similar fashion to copy the second sense strand 518 and the second antisense strand 516, respectively.

The extension 530 results in the UMI-labeled DNA 410. By way of example, the UMI-labeled DNA 410 includes a first UMI-labeled strand 532 having the first tag and the first UMI, a second UMI-labeled strand 534 having the second tag and the third UMI, a third UMI-labeled strand 536 having the first tag and the second UMI, and a fourth UMI-labeled strand 538 having the second tag and the fourth UMI. The first UMI-labeled strand 532 replicates a portion of the first sense strand 514 of the first DNA molecule 502, while the second UMI-labeled strand 534 replicates a portion of the first antisense strand 512 of the first DNA molecule 502. The first UMI-labeled strand 532 and the second UMI-labeled strand 534 include the repeat expansion region 506 having the first sequence repeat length 508. Thus, the first sequence repeat length 508 of the first DNA molecule 502 is labeled via the first UMI and the third UMI. The third UMI-labeled strand 536 replicates a portion of the second sense strand 518, and the fourth UMI-labeled strand 538 replicates a portion of the second antisense strand 516. The third UMI-labeled strand 536 and the fourth UMI-labeled strand 538 include the repeat expansion region 506 having the repeat length 510. Thus, the repeat length 510 of the second DNA molecule 504 is labeled via the second UMI and the fourth UMI.

As described above with respect to FIGS. 1 and 4, the first UMI-labeled strand 532, the second UMI-labeled strand 534, the third UMI-labeled strand 536, and the fourth UMI-labeled strand 538 may be further amplified during the second amplification reaction 414 in order to generate multiple copies of the first UMI-labeled strand 532, the second UMI-labeled strand 534, the third UMI-labeled strand 536, and the fourth UMI-labeled strand 538. In one or more implementations, sequencing adapter sequences (e.g., flow cell binding sequences), and optionally indices, are introduced during the second amplification reaction 414, resulting in the first UMI-labeled strand 532, the second UMI-labeled strand 534, the third UMI-labeled strand 536, and the fourth UMI-labeled strand 538 being prepared for sequencing (e.g., multiplex sequencing when indices are used).

### Sequence Length Distribution Analysis

FIG. 6 depicts a simplified example 600 of sequence repeat length distributions in read families targeting a variable repeat region. The simplified example 600 includes a first sequence repeat length distribution 602 corresponding to a first read family and a second sequence repeat length distribution 604 corresponding to a second read family. By way of example, the first read family may be defined as reads including a first sequence (e.g., GACTCCCCAGCA (SEQ ID NO:9)) for a forward UMI and/or a second sequence (e.g., ATAGTTGGCGAC (SEQ ID NO:10)) for a reverse UMI, and the second read family may be defined as reads including a third sequence (e.g., CTGTAAGTGCGG (SEQ ID NO:11)) as the forward UMI and/or a fourth sequence (e.g., GTACCCAGACAG (SEQ ID NO:12)) as the reverse UMI. The first sequence repeat length distribution 602 and the second sequence repeat length distribution 604 map a sequence repeat length (horizontal axis, with the length increasing from left to right) relative to count (vertical axis, with the count increasing from bottom to top). The count refers to a number of times a given sequence repeat length is found in the corresponding read family and may also be referred to as frequency.

The first sequence repeat length distribution 602 and the second sequence repeat length distribution 604 both exhibit variation of the sequence repeat length, which indicates that the sequence repeat length has been altered during amplification on some molecules (e.g., due to slippage). In the simplified example 600, the first read family represented by the first sequence repeat length distribution 602 has a consensus sequence repeat length of 19, which is the modal value of the first sequence repeat length distribution 602. Thus, the first read family is determined to have arisen through amplification of an allele having 19 sequence repeats in the targeted variable repeat region. The second read family represented by the second sequence repeat length distribution 604 has a consensus sequence repeat length of 41, which is the modal value of the second sequence repeat length distribution 604. Thus, the second read family is determined to have arisen from amplification of an allele having 41 sequence repeats in the targeted variable repeat region.

A comparison of the first sequence repeat length distribution 602 and the second sequence repeat length distribution 604 demonstrates how amplification may increase the relative representation of shorter molecules. In the simplified example 600 shown, the first read family represented by the first sequence repeat length distribution 602 is larger (e.g., includes more sequencing reads, as indicated by the higher count values) than the second read family represented by the second sequence repeat length distribution 604 due to the tendency of amplification to increase the relative representation of shorter molecules. Without the information provided by the molecular labels 126 and the subsequent analysis of the read families 140 (e.g., by the repeat length alignment module 136), the shorter molecule would be over-counted relative to its representation in the biological sample 120. However, because the sequencing reads are grouped into the read families 140 based on the molecular labels 126, the resulting read families 140 are analyzed to determine a single consensus repeat length 148 per family, regardless of the number of sequencing reads therein.

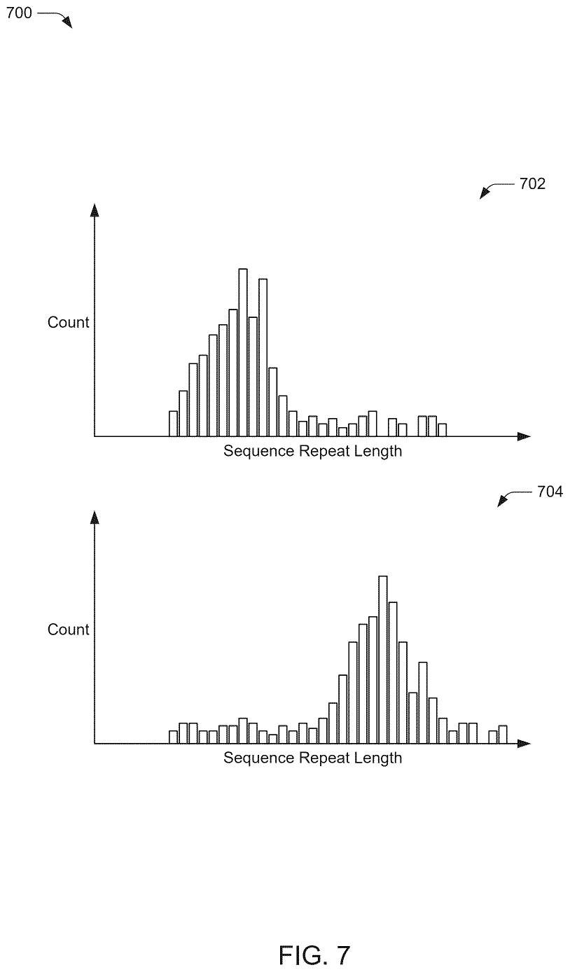

FIG. 7 depicts a simplified example 700 of sequence repeat length distributions in a biological sample. The simplified example 700 includes a first sequence repeat length distribution 702 corresponding to a first biological sample and a second sequence repeat length distribution 704 corresponding to a second biological sample. By way of example, the first biological sample and the second biological sample may be obtained from different individuals, different tissues and/or bodily fluids of a same individual, and/or may be obtained at different time points (e.g., before treatment and after treatment, prior to symptom onset and after symptom onset, before and after a pre-determined duration of time, and so forth). The first sequence repeat length distribution 702 and the second sequence repeat length distribution 704 map a sequence repeat length (horizontal axis, with the length increasing from left to right) relative to count (vertical axis, with the count increasing from bottom to top). The count refers to a number of times a given sequence repeat length is found in the corresponding biological sample. According to the techniques described herein, one count corresponds to one nucleic acid molecule of origin (e.g., from one cell of origin) in the corresponding biological sample having the corresponding sequencing repeat length. Although the first sequence repeat length distribution 702 and the second sequence repeat length distribution 704 are shown as bar graphs, other types of graphs or visualization techniques may be used. In the present example, the first sequence repeat length distribution 702 and the second sequence repeat length distribution 704 are to scale with respect to each other.

In comparing the first sequence repeat length distribution 702 and the second sequence repeat length distribution 704, the second sequence repeat length distribution 704 includes a wider sequence repeat length range than the first sequence repeat length distribution 702. For instance, the second sequence repeat length distribution 704 includes longer sequence repeat lengths that are not included in the first sequence repeat length distribution 702. The first sequence repeat length distribution 702 is skewed toward shorter sequence repeat lengths, while the second sequence repeat length distribution 704 is skewed toward longer sequence repeat lengths. That is, shorter sequence repeat lengths occur more frequently than longer sequence repeat lengths in DNA isolated from the first biological sample, whereas longer sequence repeat lengths occur more frequently than shorter sequence repeat lengths in DNA isolated from the second biological sample.

As illustrative examples, the first sequence repeat length distribution 702 and the second sequence repeat length distribution 704 may be used during repeat expansion disorder diagnosis, to classify individuals for inclusion or exclusion in clinical trials, to evaluate treatment outcomes, for identifying mechanisms of pathology, and the like. As such, producing highly accurate sequence repeat length distributions using genomic DNA samples facilitates a wide variety of clinical and research applications for repeat expansion disorders.

Having discussed example details of the techniques for the analysis of repeat expansion disorders, consider now an example procedure to illustrate additional aspects of the techniques.

### Example Procedures

This section describes example procedures for the analysis and treatment of repeat expansion disorders in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, at least portions of the procedures are performed by a suitably configured device, such as the sequencing data processor 110 of FIG. 1, by executing instructions stored in a non-transitory computer-readable storage medium.

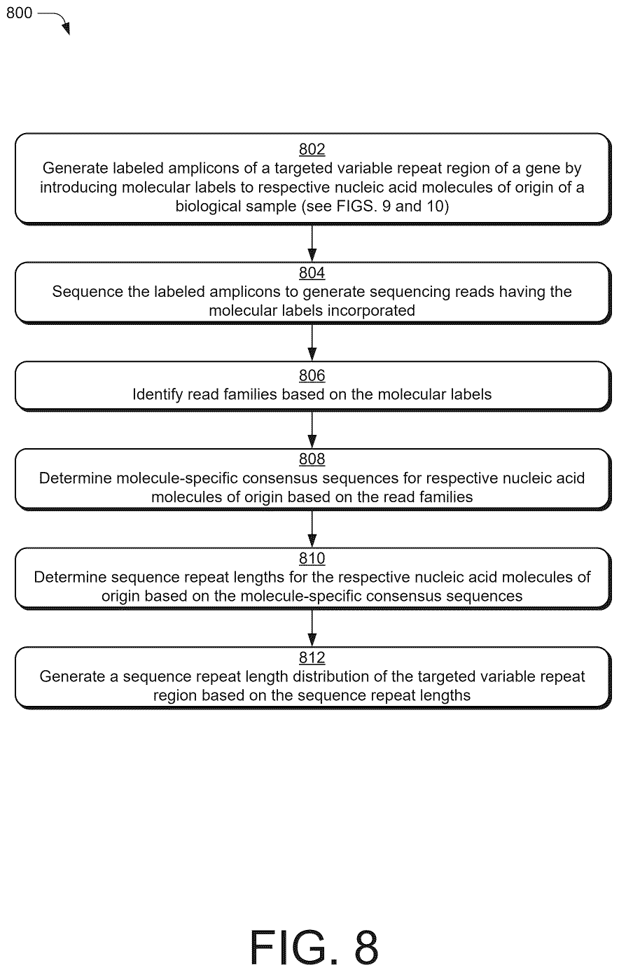

FIG. 8 depicts an example procedure 800 in which sequence length distribution analysis is performed. The procedure 800 provides a high-level method that may be applied to samples prepared via single cell/nucleus sequencing or genomic DNA sequencing, for instance.

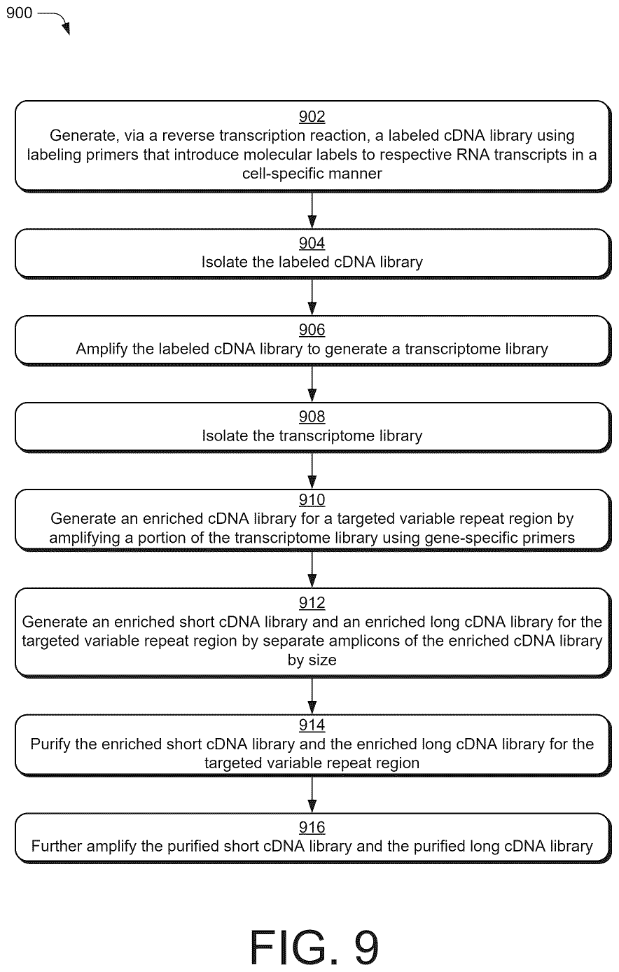

Labeled amplicons of a targeted variable repeat region of a gene are generated by introducing molecular labels to respective nucleic acid molecules of origin from a biological sample (block 802). By way of example, the molecular labels (e.g., the molecular labels 126) may be introduced via primers (e.g., the primers 124 of FIG. 1) used in one or more reverse transcription and/or amplification reactions performed at a nucleic acid amplifier (e.g., the nucleic acid amplifier 106 of FIG. 1). In at least one implementation, the labeled amplicons are generated from RNA transcripts, and the molecular labels include cell barcodes (e.g., the cell barcodes 128 of FIG. 1) and UMIs (e.g., the UMIs 130 of FIG. 1). As described above with respect to FIGS. 1-3, the cell barcodes may distinguish amplicons derived from one cell from another, and the UMIs may uniquely label amplicons derived from different RNA transcripts of origin. Generation of the labeled amplicons in example single cell/nucleus sequencing implementations will be further described below with reference to FIG. 9.

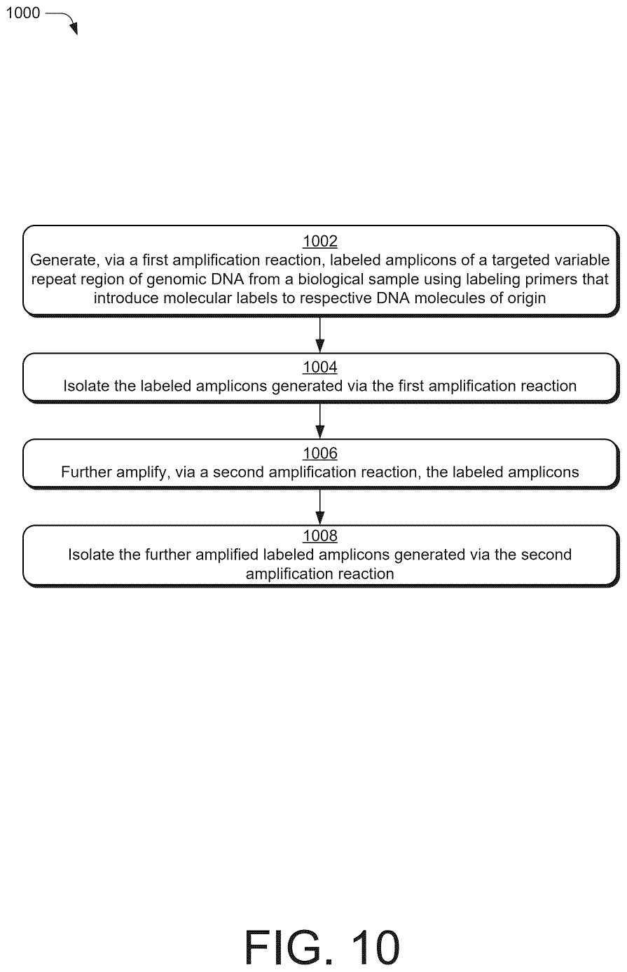

In at least one variation, the labeled amplicons are generated from genomic DNA, and the molecular labels include the UMIs. As described above with respect to FIGS. 1, 4, and 5, the UMIs may uniquely label amplicons derived from different DNA molecules of origin. Generation of the labeled amplicons in example genomic DNA sequencing implementations will be further described below with reference to FIG. 10.

In at least one implementation, the molecular labels further include indices (e.g., the indices 132 of FIG. 1) to enable sequencing multiplexing to be performed. When used, the indices 132 include one or more (e.g., two) short sequences having a known order of nucleotides that is used to distinguish one sample from another.

The labeled amplicons are sequenced to generate sequencing reads having the molecular labels incorporated (block 804). By way of example, a DNA sequencer (e.g., the DNA sequencer 108 of FIG. 1) may use a long read sequencing technique that produces long reads (e.g., sequencing data) that typically range from 2000 bases to 1,000,000 bases and more typically from 5000 bases to 800,000 bases in length. Alternatively, the DNA sequencer may use a short read sequencing technique that produces short reads typically ranging from approximately 10 bases to approximately 600 bases and more typically from approximately 50 bases to approximately 800 bases. The sequencing reads include an ordered combination of nucleotides (e.g., adenine, thymine, cytosine, and guanine, abbreviated as A, T, C, and G, respectively).

Read families are identified based on the molecular labels (block 806). By way of example, a repeat length alignment module (e.g., the repeat length alignment module 136 of FIG. 1) uses one or more read family identification algorithms (e.g., the one or more read family identification algorithms 138 of FIG. 1, which include statistical and/or computational algorithms/models) to determine which sequencing reads correspond to a same nucleic acid molecule of origin in the biological sample based on the molecular labels (e.g., the UMIs and the cell barcodes and/or indices, when included). A given read family comprises sequence fragments (e.g., reads) having a matching UMI or pair of UMIs (e.g., one forward labeling primer UMI and one reverse labeling primer UMI in some genomic DNA sequencing implementations). When cell barcodes are used in single cell sequencing experiments, the given read family further comprises reads having a same cell barcode sequence. When indices are used for multiplexed sequencing, the given read family additionally comprises reads having a same index or pair of indices (e.g., one forward amplification primer index sequence and one reverse amplification primer index sequence). For instance, the one or more read family identification algorithms may sort the sequencing data by the indices to distinguish reads from one sample from another in a multiplexed sequencing reaction, when used. The reads identified for a given index or dual index may be further sorted based on the sequences of the cell barcodes, when used, and then based on the UMIs so that sequencing reads having a common UMI sequence (or pair of UMI sequences) are grouped to generate the read families.

Molecule-specific consensus sequences for respective DNA molecules of origin are determined based on the read families (block 808). By way of example, the repeat length alignment module 136 uses one or more alignment algorithms (e.g., the one or more alignment algorithms 142 of FIG. 1) to map the sequencing reads of a given read family with respect to each other, thus generating a read family alignment (e.g., the read family alignments 144 of FIG. 1). The one or more alignment algorithms 142 may include functionality for finding an alignment that increases (e.g., maximizes) a similarity between reads of the given read family using a scoring system that considers possible insertions, deletions, and mismatches that may arise during amplification (e.g., due to a fidelity of a polymerase enzyme) or sequencing (e.g., due to base calling errors). A molecule-specific consensus sequence (e.g., the molecule-specific consensus sequence 146 of FIG. 1) for the given read family may include nucleotides present in a majority of read sequences at a specific position to be chosen (e.g., by the alignment module) for the consensus sequence at that position.

Sequence repeat lengths for the respective DNA molecules of origin are determined based on the molecule-specific consensus sequences (block 810). By way of example, the repeat length alignment module 136 may infer the sequence repeat length for a given DNA molecule of origin based on a number of sequence repeats in the targeted variable repeat region, as indicated by the molecule-specific consensus sequence and/or a distribution of the sequence repeat lengths in the corresponding read family. In at least one implementation, the repeat length alignment module 136 may identify the variable repeat region without user input by analyzing the molecule-specific consensus sequences and identifying a sequence repeat (e.g., a dinucleotide repeat, a trinucleotide repeat, a tetranucleotide repeat, a pentanucleotide repeat, or another unit of nucleotide repeat) that is consecutively repeated a plurality of times. In at least one variation, however, the repeat length alignment module 136 receives user input indicating the sequence repeat (e.g., “CAG”) and/or the position of the targeted variable repeat region, such as defined based on expected sequence(s) flanking the targeted variable repeat region.

The sequence repeat length refers to a number of times the sequence repeat (e.g., the unit of nucleotides) is consecutively repeated in the targeted variable repeat region. As a non-limiting, illustrative example, a sequence repeat length of 50 corresponds to the molecule-specific consensus sequence having 50 consecutive repeats of the sequence repeat. As another non-limiting, illustrative example, a sequence repeat length of 350 corresponds to the molecule-specific consensus sequence having 350 consecutive repeats of the sequence repeat.

A sequence repeat length distribution of the targeted variable repeat region is generated based on the sequence repeat lengths (block 812). By way of example, because the targeted variable repeat region is somatically unstable, variation in the sequence repeat length is expected in the biological sample. Nucleic acid originating from one cell, for instance, may have a different number of consecutive repeats compared to nucleic acid originating from another cell, even if collected from a same tissue or bodily fluid. As such, the sequence repeat length distribution (e.g., the sequence repeat length distribution 152 of FIG. 1) indicates a range of sequence repeat lengths (e.g., from a minimum sequence repeat length value to a maximum sequence repeat length value) found in the biological sample and a frequency of individual sequence repeat lengths within this range. In this way, the sequence repeat length distribution indicates whether longer or shorter lengths occur more frequently in the biological sample, whether the range is larger or smaller, and whether particularly long lengths are found, which may inform on disease progression of a repeat expansion disorder and/or an efficacy of a therapeutic intervention.

Because the molecule-specific consensus sequences are used to determine the sequence repeat lengths, and the sequence repeat length distribution therefrom, the sequence length distribution is not skewed based on a bias of the amplification reaction toward shorter molecules in a bulk setting. That is, even though shorter amplicons may be generated in larger quantities than longer amplicons, a single consensus sequence is generated for a given amplicon based on the unique molecular label incorporated therein. As a result, the sequence repeat length distribution provides an accurate representation of the somatic instability of the variable repeat region.

FIG. 9 depicts an example procedure 900 in which a single cell/nucleus RNA sequencing sample is prepared for sequence length distribution analysis. The procedure 900 may be performed prior to the procedure 800 of FIG. 8, for instance.

A labeled cDNA library is generated via a reverse transcription reaction using labeling primers that introduce molecular labels to respective RNA transcripts in a cell-specific manner (block 902). By way of example, the reverse transcription reaction may be performed at a thermal cycler (e.g., the nucleic acid amplifier 106 of FIG. 1), and the labeling primers may be configured to target RNA transcripts through complementary base pairing. The labeling primers, for instance, may include a plurality of primer molecules that each include a unique molecular identifier (UMI) and a cell barcode. The labeling primers provided to a single cell, for instance, may include a same cell barcode sequence and different UMI sequences respect to each other. During the reverse transcription reaction, cDNA synthesis extends from the labeling primer, resulting in the synthesis of a DNA segment (e.g., an amplicon) that is appended with the cell barcode and the UMI. The cell barcodes enable cDNA derived from different cells to be distinguished from each other, while the UMIs enable cDNA derived from different RNA transcripts to be distinguished from each other.

The labeling primer molecules may further include a common adapter sequence, e.g., at a 5′ end of the labeling primer molecules, which is targeted in a subsequent amplification reaction (e.g., occurring after the reverse transcription reaction), as will be elaborated below, e.g., at block 906. The molecular labels (e.g., the cell barcodes 128 and the UMIs 130) may be positioned between an RNA targeting sequence (e.g., an oligo dT) and the common adapter sequence.

The reverse transcription reaction is performed in such a way as to introduce one pair of molecular labels (e.g., one UMI and one cell barcode) to cDNA derived from respective RNA transcripts of origin. By way of example, the reverse transcription reaction uses nuclei encapsulation and bead-bound primers in order to introduce one cell barcode sequence per cell, and each primer bound to a given bead may include a different UMI sequence.

The labeled cDNA library is isolated (block 904). By way of example, following the reverse transcription reaction, the labeled amplicons are in a mixture with reagents used in the reverse transcription reaction, including the RNA transcripts, the labeling primers, nucleotides, reverse transcriptase enzyme, and buffer. Therefore, a clean-up technique is used to isolate the labeled amplicons from the reagents used in the reverse transcription reaction. Example clean-up techniques include spin-column purification and bead-based purification, such as described above with respect to FIG. 2A, although other clean-up techniques that selectively capture and then elute the labeled cDNA may be employed.

The cDNA library is further amplified to generate a transcriptome library (block 906). By way of example, a transcriptome amplification reaction may be performed using cDNA primers that target the common adapter sequences introduced via the labeling primers used in the reverse transcription reaction, resulting in the labeled cDNA being further amplified. The cDNA primers may be generic cDNA primers that target the 5′ and 3′ sequence adapters of the cDNA molecules, e.g., the common adapter and a TSO adapter. While the cDNA primers may be non-specific to a gene of interest, in one or more implementations, spike-in primers targeting a variable repeat region of the gene of interest are also used in order to increase a yield of the targeted repeat expansion region. The transcriptome amplification reaction is performed in the thermal cycler in a manner that amplifies the labeled cDNA for substantially all RNA transcripts of origin, thus generating the transcriptome library (e.g., the transcriptome library 228 of FIGS. 2A and 2B).

The transcriptome library is isolated (block 908). By way of example, following the transcriptome amplification reaction, the transcriptome library is in a mixture with reagents used in the transcriptome amplification reaction, including the amplification primers, the nucleotides, the polymerase enzyme, and the buffer. Therefore, a second clean-up is performed to isolate the labeled amplicons from the reagents used in the transcriptome amplification reaction. The second clean-up may use the same or a different technique than that used following the reverse transcription reaction. In at least one implementation, solid phase reversible immobilization (SPRI) may be used, where paramagnetic beads are used to selectively bind DNA fragments of a selected size range while the primers, unused nucleotides, enzymes, salts, etc. are washed away. An example SPRI procedure is described with respect to FIG. 2A.

An enriched cDNA library is generated for the targeted variable repeat region by amplifying a portion of the transcriptome library using gene-specific primers (block 910). By way of example, a first portion of the transcriptome library may be saved for whole transcriptome analysis, and a second portion of the transcriptome library may be used to generate the enriched cDNA library via a targeted amplification reaction (e.g., the targeted amplification reaction 230 of FIG. 2A). The gene-specific primers may include a small molecule-tagged (e.g., biotinylated) primer designed to anneal to the 5′ end of the targeted variable repeat region and another primer designed to target the common adapter added during the reverse transcription. The gene-specific primers facilitate selective amplification of the targeted variable repeat region, and the small molecule tag may enable subsequent purification, such as will be described below with respect to block 914.

In at least one implementation, the gene-specific primers further include adapter sequences that may be targeted during a subsequent amplification region. The subsequent amplification reaction, which will be described below with respect to block 916, may be used to introduce indices and/or sequencing adapters as well as generate a sufficient quantity of cDNA for sequencing, for example.

An enriched short cDNA library and an enriched long cDNA library are generated by separating amplicons of the enriched cDNA library by size (block 912). By way of example, an SPRI technique may be used to separate the amplicons of the enriched cDNA library by size. Alternatively, another type of size selection technique may be used. The short enriched cDNA library (e.g., the short enriched cDNA library 240 of FIG. 2B) comprises target-enriched amplified barcoded and UMI-labeled cDNA molecules having shorter molecular lengths, and the long enriched cDNA library (e.g., the long enriched cDNA library 242 of FIG. 2B) comprises target-enriched barcoded and UMI-labeled cDNA molecules having longer molecular lengths. Size separation may reduce or prevent the effects of length bias in a subsequent amplification reaction, for instance.

The enriched short cDNA library and the enriched long cDNA library are purified for the targeted variable expansion region (block 914). By way of example, when the gene-specific primers (e.g., block 910) include a biotinylated primer, the enriched short cDNA library and the enriched long cDNA library may be purified via affinity purification using streptavidin beads. The streptavidin beads bind the biotin molecule, thus selectively binding the cDNA constructs of the targeted variable expansion region and enabling other cDNA constructs to be removed.

The purified short cDNA library and the purified long cDNA library are further amplified (block 916). In at least one implementation, the purified short cDNA library (e.g., the short target cDNA library 246 of FIG. 2B) and the purified long target cDNA library (e.g., the long target cDNA library 248 of FIG. 2B) are further amplified and/or indexed for sequencing via an additional amplification reaction. By way of example, the additional amplification reaction (e.g., the additional amplification reaction 250 of FIG. 2B) uses separate reaction mixtures for the purified short cDNA library and the purified long cDNA library. The additional amplification reaction optionally incorporates indices (e.g., the indices 132 of FIG. 1). Single indexing (where one index is incorporated) or dual indexing (where two indices are incorporated) may be used. The dual indexing may be unique dual indexing or combinatorial dual indexing, for example. The indices may be short (e.g., 8-12 nucleotide) sequences that are assigned to a given sample to be sequenced in order to provide an identifying label for the sample for multiplexed sequencing. However, it is to be appreciated that the indices may be omitted, such as when multiplexed sequencing is not used. The primers used in the additional amplification reaction may further append sequencing adapters that enable flow cell binding during a subsequent sequencing process. In at least one implementation, the primers used in the additional amplification reaction (e.g., the amplification primers 254 of FIG. 2B) target adapter sequences added via the gene-specific primers of the targeted amplification reaction of block 910.

The purified short DNA library and the purified long cDNA library then be sequenced, e.g., according to the procedure 800 of FIG. 8, in order to generate a sequence repeat length distribution of the targeted variable repeat region, such as described above. As a result, the sequence repeat length distribution provides an accurate representation of the somatic instability of the variable repeat region with single-cell resolution, which may be further compared to genome-wide gene expression changes using sequencing data from the transcriptome library.

FIG. 10 depicts an example procedure 1000 in which a genomic DNA sample is prepared for sequence length distribution analysis. The procedure 1000 may be performed prior to the procedure 800 of FIG. 8, for instance.

Labeled amplicons of a targeted variable repeat region of genomic DNA from a biological sample are generated via a first amplification reaction using labeling primers that introduce molecular labels to respective DNA molecules of origin (block 1002). By way of example, the first amplification reaction may be a polymerase chain reaction performed at a thermal cycler (e.g., the nucleic acid amplifier 106 of FIG. 1), and the labeling primers may be configured to target regions of DNA flanking the targeted variable repeat region through complementary base pairing. A forward labeling primer, for instance, is designed to anneal to a region upstream of the targeted variable repeat region, and a reverse labeling primer is designed to anneal to a region downstream of the targeted variable repeat region. During the first amplification reaction, DNA synthesis extends from the forward and reverse labeling primers in opposite directions, resulting in the amplification of a DNA segment (e.g., an amplicon) located between the two labeling primers. Because the labeling primers flank the targeted variable repeat region, the DNA segment includes the targeted variable repeat region.

In at least one implementation, the forward labeling primer and the reverse labeling primer both include a molecular label, e.g., a unique molecular identifier (UMI). In at least one variation, however, one of the forward labeling primer and the reverse labeling primer does not include the molecular label. The molecular label includes a short sequence of random nucleotides that is used for one forward labeling primer molecule and/or one reverse labeling primer molecule. The molecular label, for instance, serves as a barcode to distinguish amplicons generated from one DNA molecule of origin from those generated from another DNA molecule of origin.

By way of example, the forward labeling primers include a collection of forward labeling primer molecules that have different sequences for the molecular label with respect to each other. The forward labeling primer molecules further include a common (e.g., shared by the forward labeling primer molecules) target binding sequence (e.g., a locus-specific sequence) configured to anneal to the region upstream of the targeted variable repeat region. The common target binding sequence of the forward labeling primer, for instance, may be positioned at a 3′ end of the forward labeling primer molecules. The forward labeling primer molecules may further include a common forward tag sequence, e.g., at a 5′ end of the forward labeling primer molecules, which is targeted in a subsequent amplification reaction (e.g., occurring after the first amplification reaction), as will be elaborated below, e.g., at block 1006. The molecular label (e.g., the short sequence of random nucleotides) may be positioned between the common target binding sequence and the common forward tag sequence.

Similarly, the reverse labeling primers may include a collection of reverse labeling primer molecules that have different sequences for the molecular label with respect to each other. The reverse labeling primer molecules further include, at a 3′ end, a common (e.g., shared by the reverse labeling primer molecules) target binding sequence (e.g., another locus-specific sequence) configured to anneal to the region downstream of the targeted variable repeat region and, at a 5′ end, a common reverse tag sequence that is targeted in a subsequent amplification reaction. The target binding sequence and the reverse tag sequence of the reverse labeling primers are different than those of the forward labeling primers. Like the forward labeling primers, the molecular label of the reverse labeling primers may be positioned between the common target binding sequence and the common reverse tag sequence.

The first amplification reaction is performed in such a way as to introduce one pair of molecular labels (e.g., one forward labeling primer molecule and one reverse labeling primer molecule) to a respective DNA molecule of origin. By way of example, the first amplification reaction includes a small number of reaction cycles, such as a number of reaction cycles between one and five. As further described herein, e.g., with respect to FIG. 4, a reaction cycle may include a DNA denaturation step performed at a first temperature for a first amount of time followed by an annealing step performed at a second temperature for a second amount of time, which is further followed by an extension step performed at a third temperature for a third amount of time.

Because newly synthesized DNA of the targeted variable repeat region extends from the primers, a resulting amplicon includes the molecular labels included within the forward and/or reverse primers, e.g., the labeled amplicons. For instance, when the forward labeling primer and the reverse labeling primer both include molecular labels, a pair of molecular labels is associated with a DNA segment amplified from a given DNA molecule of origin. In at least one variation where one of the forward and the reverse labeling primers does not include the molecular label, one molecular label is associated with the DNA segment of the given DNA molecule.

The labeled amplicons generated via the first amplification reaction are isolated (block 1004). By way of example, following the first amplification reaction, the labeled amplicons are in a mixture with reagents used in the first amplification reaction, including the genomic DNA, the labeling primers, nucleotides, polymerase enzyme, and buffer. Therefore, a clean-up technique is used to isolate the labeled amplicons from the reagents used in the first amplification reaction. Example clean-up techniques include spin-column purification and SPRI, such as described above with respect to FIG. 4, although other clean-up techniques that selectively capture and then elute the labeled amplicons may be employed. For instance, in at least one variation, the forward primer or the reverse primer additionally includes a small molecule (e.g., biotin) at the 5′ end that selectively binds to an affinity purification agent (e.g., avidin or streptavidin coated beads or columns), enabling the first amplification reaction reagents to be removed before the labeled amplicons are eluted from the affinity purification agent.

The labeled amplicons are further amplified via a second amplification reaction (block 1006). By way of example, amplification primers may be used that target the tag sequences introduced via the labeling primers used in the first amplification reaction, resulting in the labeled amplicons being further amplified. In one or more implementations, the amplification primers introduce additional sequence(s) that further prepare the labeled amplicons for sequencing (e.g., multiplexed sequencing). The amplification primers, for instance, may introduce one or more indices and/or sequencing adapters to the labeled amplicons. In at least one implementation, dual indexing is used, where a forward amplification primer and a reverse amplification primer both include an index sequence. Unlike the forward labeling primer molecules and the reverse labeling primer molecules used in the first amplification reaction, forward amplification primer molecules used in a given amplification reaction have the same sequence with respect to each other, and reverse amplification primer molecules used in the given amplification reaction have the same sequence with respect to each other. For instance, different index sequences are used to distinguish amplicons derived from different biological samples from each other, rather than to distinguish between different DNA molecules of origin within a same biological sample.

In one or more implementations, a 3′ region of the forward amplification primer anneals to the common forward tag sequence of the forward labeling primer, and a 3′ region of the reverse amplification primer anneals to the common reverse tag sequence of the reverse labeling primer. Moreover, a 5′ region of the forward amplification primer may include a first sequencing adapter sequence, and a 5′ region of the reverse amplification primer may include a second sequencing adapter sequence. The indices may be positioned between the corresponding tag annealing and sequencing adapter sequences, when included. Sequences of the indices of the amplification primers used in the second amplification reaction are known. This enables sequencing reads corresponding to one biological sample to be distinguished from those of another biological sample in a multiplexed sequencing reaction.

The second amplification reaction is performed in the thermal cycler in a manner that introduces the indices to the labeled amplicons, resulting in indexed and labeled amplicons that are adapted for sequencing. Moreover, the second amplification reaction includes more reaction cycles than the first amplification reaction in order to amplify and generate many more copies of the indexed and labeled amplicons. By way of example, the second amplification reaction includes a relatively large number of reaction cycles, such as a number of reaction cycles between six and forty. Temperature and time settings used for the reaction cycles in the second amplification reaction may be the same as or different than those used for the first amplification reaction.

The further amplified labeled amplicons generated via the second amplification reaction are isolated (block 1008). By way of example, following the second amplification reaction, the further amplified labeled amplicons are in a mixture with reagents used in the second amplification reaction, including the amplification primers, the nucleotides, the polymerase enzyme, and the buffer. Therefore, a second clean-up is performed to isolate the labeled amplicons from the reagents used in the second amplification reaction. The second clean-up may use the same or a different technique than that used following the first amplification reaction. In at least one implementation, more than one clean-up technique is used. In addition to or as an alternative to the clean-up techniques discussed above at block 1004, gel electrophoresis and subsequent band excision and extraction may be used following the second amplification reaction. By way of example, due to the larger number of reaction cycles, which result in a larger quantity of DNA being generated, there may be sufficient quantities of DNA present to enable the indexed and labeled amplicons to resolve into a UV-visible band in a UV-stained agarose gel.

The labeled amplicons may then be sequenced, e.g., according to the procedure 800 of FIG. 8, in order to generate a sequence repeat length distribution of the targeted variable repeat region, such as described above. As a result, the sequence repeat length distribution provides an accurate representation of the somatic instability of the variable repeat region without using a time consuming and technically complex single-cell analysis.

Having described example procedures in accordance with one or more implementations, consider now an example system and device that can be utilized to implement the various techniques described herein.

### Example System and Device

FIG. 11 illustrates an example system generally at 1100 that includes an example computing device 1102 that is representative of one or more computing systems and/or devices that may implement the various techniques described herein. This is illustrated through inclusion of the sequencing data processor 110. The computing device 1102 may be, for example, a server of a service provider, a device associated with a client (e.g., a client device), an on-chip system, and/or any other suitable computing device or computing system.

The example computing device 1102 as illustrated includes a processing system 1104, one or more computer-readable media 1106, and one or more I/O interfaces 1108 that are communicatively coupled, one to another. Although not shown, the computing device 1102 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionality to perform one or more operations using hardware. Accordingly, the processing system 1104 is illustrated as including hardware elements 1110 that may be configured as processors, functional blocks, and so forth. This may include implementation in hardware as an application specific integrated circuit or other logic device formed using one or more semiconductors. The hardware elements 1110 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 1106 is illustrated as including memory/storage 1112. The memory/storage 1112 represents memory/storage capacity associated with one or more computer-readable media. The memory/storage 1112 may include volatile media (such as random-access memory (RAM)) and/or nonvolatile media (such as read only memory (ROM), Flash memory, optical disks, magnetic disks, and so forth). The memory/storage 1112 may include fixed media (e.g., RAM, ROM, a fixed hard drive, and so on) as well as removable media (e.g., flash memory, a removable hard drive, an optical disc, and so forth). The computer-readable media 1106 may be configured in a variety of other ways as further described below.

Input/output interface(s) 1108 are representative of functionality to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1102. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1110. The computing device 1102 may be configured to implement particular instructions and/or functions corresponding to the software and/or hardware modules. Accordingly, implementation of a module that is executable by the computing device 1102 as software may be achieved at least partially in hardware, e.g., through use of computer-readable storage media and/or hardware elements 1110 of the processing system 1104. The instructions and/or functions may be executable/operable by one or more articles of manufacture (for example, one or more computing devices 1102 and/or processing systems 1104) to implement techniques, modules, and examples described herein.

The techniques described herein may be supported by various configurations of the computing device 1102 and are not limited to the specific examples of the techniques described herein. This functionality may also be implemented all or in part through use of a distributed system, such as over a “cloud” 1114 via a platform 1116 as described below.

The cloud 1114 includes and/or is representative of a platform 1116 for resources 1118, which are depicted including the sequencing data processor 110. The platform 1116 abstracts underlying functionality of hardware (e.g., servers) and software resources of the cloud 1114. The resources 1118 may include applications and/or data that can be utilized while computer processing is executed on servers that are remote from the computing device 1102. Resources 1118 can also include services provided over the Internet and/or through a subscriber network, such as a cellular or Wi-Fi network.

The platform 1116 may abstract resources and functions to connect the computing device 1102 with other computing devices. The platform 1116 may also serve to abstract scaling of resources to provide a corresponding level of scale to encountered demand for the resources 1118 that are implemented via the platform 1116. Accordingly, in an interconnected device example, implementation of functionality described herein may be distributed throughout the system 1100. For example, the functionality may be implemented in part on the computing device 1102 as well as via the platform 1116 that abstracts the functionality of the cloud 1114.

Having discussed example details of the techniques for sequence repeat length distribution analysis, consider now the following examples to illustrate usage of the techniques in the context of repeat expansion disorders.

### EXAMPLE APPLICATIONS

### Example 1: Using Single Cell Sequencing to Investigate how Acquired DNA Repeat Expansion Drives Striatal Neuropathology in Huntington's Disease

To better understand the pathophysiological process in Huntington's Disease (HD), droplet-based single-nucleus RNA-seq according to the workflow 200 outlined in FIGS. 2A-2B was used to measure RNA expression in more than one million individual nuclei sampled from the caudate nucleus (the largest component of the striatum) of 56 persons with HD and 53 unaffected individuals (e.g., age-matched controls). A length of an HTT-CAG repeat, the repeat expansion region involved in HD, was measured at single-cell resolution alongside the same cells' genome-wide RNA expression, e.g., according to the workflow 200, thus enabling the length of the HTT-CAG repeat to be related to cell types and their biological states.

### Single-Nucleus RNA-Seq Analysis of HD

The anterior caudate (obtained postmortem) from persons with HD (pwHD) and age-matched controls were analyzed. Affected individuals were sampled so as to represent a wide range of stages in the progression of HD, from “at-risk” gene-expansion carriers who passed away before symptom onset, to individuals with incipient clinical symptoms at time of death but no detected neuropathology (Vonsattel grade 0), to individuals with advanced caudate neurodegeneration (Vonsattel grade 4).

To make rigorous comparisons of nuclei from many brain donors while controlling for technical influences from nuclear extraction, single-cell library construction, and sequencing, preparations of nuclei were created from pools of 20 donors at once, with nuclei isolated from similar masses of caudate tissue from each donor. Nuclei from each 20-donor pool were processed together as a single sample through nuclear extraction, encapsulation in droplets (e.g., the first droplet 320 and the second droplet 326 of FIG. 3), and in creating and sequencing the resulting snRNA-seq libraries (e.g., the transcriptome library 228). Combinations of transcribed single nucleotide polymorphisms (SNPs) in each cell's sequencing data 122 were used to assign each nucleus to its donor-of-origin. This “genetic multiplexing” approach allows the sequencing data 122 to be highly comparable donor-to-donor.

A large quantity of nuclei (e.g., ˜613,000) may be analyzed by this approach. Each nucleus was readily assigned to one of seven major cell classes, based on its genome-wide pattern of RNA expression.

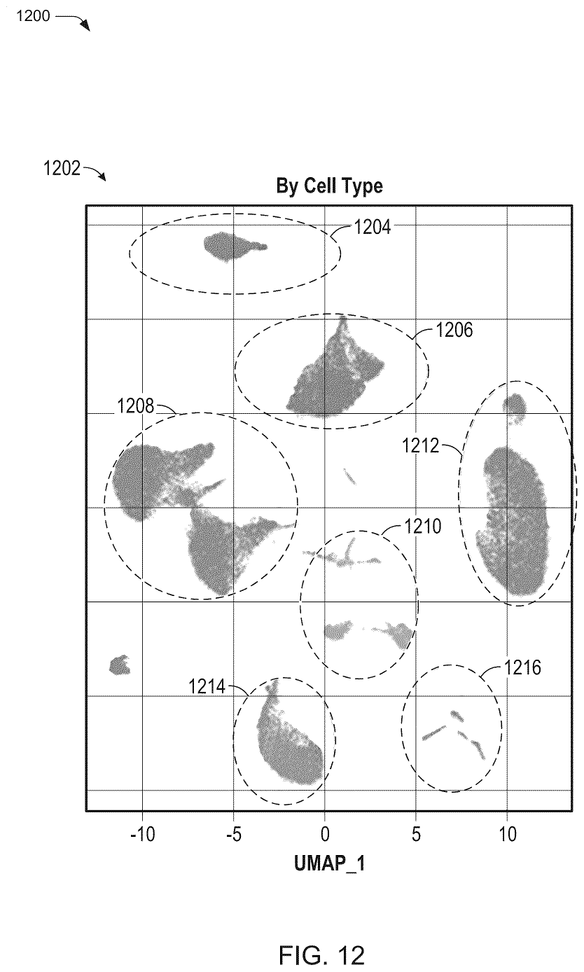

FIG. 12 shows an example 1200 genome-wide pattern of RNA expression as assigned by cell type, as shown in a projection 1202. In the example 1200, 613,000 nuclei were sampled from the anterior part of the caudate nucleus—the largest component of the striatum, and the region with the most cell death in HD—from 56 persons with HD and 53 unaffected controls (mean 5,630 cell nuclei per donor). Each nucleus was assigned to one of seven major cell classes based on the RNAs it expressed, as indicated in the projection 1202. Data points of the projection 1202 correspond to a single nucleus RNA expression profile. In the projection 1202, data points that are similar are positioned closer together, with a degree of closeness corresponding to a degree of similarity, and data points that are dissimilar are positioned farther apart in a similar fashion. The projection 1202 generally includes a polydendrocyte cluster 1204, an oligodendrocyte cluster 1206, a striatal projection neuron (SPN, also called medium spiny neuron or MSN) cluster 1208, an interneuron cluster 1210, an astrocyte cluster 1212, a microglia cluster 1214, and an endothelia cluster 1216. As such, the projection 1202 enables each nucleus to be assigned as a polydendrocyte, an oligodendrocyte, an SPN, an interneuron, an astrocyte, microglia, or endothelia based on the position of its data point with respect to the clusters. As an illustrative example, a given nucleus may be assigned as an SPN in response to its data point being positioned in the SPN cluster 1208.

### Cell-Type-Specific Vulnerability in HD

The vulnerability of SPNs in relation to each donor's age and the length of their inherited HTT allele was evaluated. By way of example, caudate atrophy in HD involves loss of SPNs (the caudate's principal neuronal population), while sparing other types of neurons and glia in the caudate. In tissue from control donors, 46% (+/−6%) of the nuclei sampled were derived from SPNs. This fraction was greatly reduced in most persons with HD.

To analyze together persons with many different ages and inherited CAG repeat lengths, a CAG-Age-Product (CAP) score, which is a common estimate of onset and progression in HD, was used. The CAP score is a mathematical function of age and inherited CAG length (calculated as age*inherited CAG length−33.6) that is routinely used to provide prognostic information to pwHD and to identify candidate patients for clinical trials. Higher CAP scores represent later disease stages; the centrality of inherited CAG length in the formula reflects the well-established finding that longer inherited alleles result in earlier onset and faster progression. In the present analysis, the use of CAP score allows persons with many different ages and inherited CAG repeat lengths to be combined into a single analysis.

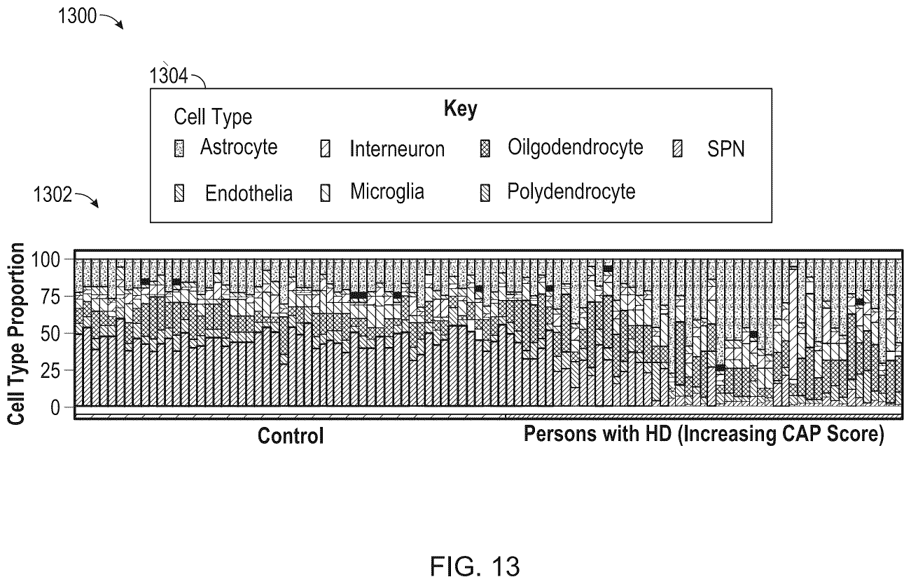

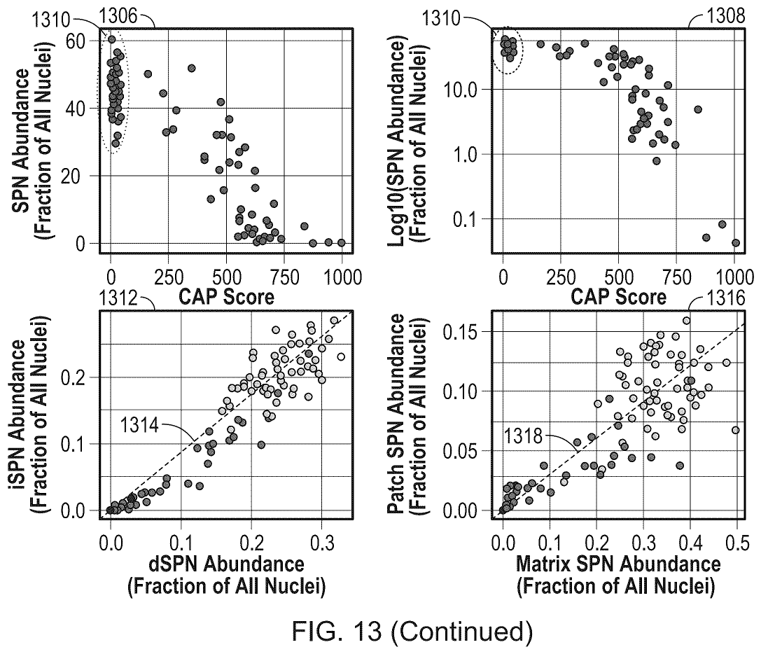

FIG. 13 shows an example 1300 of SPN abundance in relation to CAP scores. A diagram 1302 shows caudate cell-type proportions in each donor, show the loss of SPNs in persons with HD. In the figure, control donors are in the left half, and persons with HD are ordered from left to right by increasing CAP score. A key 1304 indicates cell type, dark bar portions corresponding to SPNs positioned at the bottom of the diagram 1302 while dark par portions corresponding to astrocytes are positioned at the top of the diagram 1302. A first plot 1306 relates CAP score (horizontal axis) to the number of SPNs as a fraction of nuclei on a linear scale (vertical axis), while a second plot 1308 relates CAP score (horizontal axis) to the number of SPNs as a fraction of nuclei on a log scale (vertical axis). As shown in the first plot 1306, the abundance of SPN nuclei in the anterior caudate (as a fraction of all nuclei) exhibited a clear decline in relationship to increasing CAP score. Persons with CAP scores up to 300 (corresponding to 37 year old in a donor with an inherited 42-CAG allele) tended to have SPN proportions within the range sampled in controls, which are highlighted in a control cluster 1310 on each of the first plot 1306 and the second plot 1308. These results are consistent with findings that caudate atrophy commences subtly about 10-15 years before the onset of motor symptoms, then greatly escalates. Loss of SPNs appeared to greatly accelerate in donors with CAP score greater than 300 (donors with manifest HD symptoms), as evidenced by an increasingly steep downward slope in the relationship of SPN abundance to CAP score. Persons with CAP scores up to 300 (generally corresponding to the long latent period prior to clinical motor onset) tended to have SPN proportions just slightly lower than the average unaffected control brain donor. Almost all persons with HD with CAP scores of greater than 600 had just a small fraction of the SPNs present in control donors.

The second plot 1308 considers the abundance of SPNs on a log-scale against disease progression in order to estimate how the cell-intrinsic vulnerability of SPNs (their rate or probability of loss) changes over time. The slope of the resulting curve is modest before HD onset (e.g., across CAP scores of 0-300), then becomes steeply negative as CAP scores further increase. This downward slope does not attenuate as the CAP scores further increases, suggesting that SPN vulnerability remains high throughout HD progression (and inconsistent with a longstanding idea that surviving SPNs might be a “resilient” subpopulation).

The relative vulnerabilities of subtypes of SPNs were assessed, as indicated in a third plot 1312. Two canonical SPN types are defined by their connectivity and gene expression and include direct-pathway SPNs (dSPNs, which express DRD1 and are also called D1-SPNs) and indirect-pathway SPNs (iSPNs, which express DRD2 and are also called D2-SPNs). iSPNs and dSPNs are readily distinguished from each other based on their genome-wide RNA expression patterns. iSPNs comprised approximately 47% of the SPN population in controls, but a smaller fraction in pwHD, indicating that iSPNs tend to become vulnerable more quickly (on average) than dSPNs do. Since iSPNs inhibit motor programs while dSPNs initiate them, the faster early loss of iSPNs might underlie the prominence of chorea (involuntary movements) as an early motor symptom, before paralysis becomes the dominant motor symptom in HD.

For example, the third plot 1312 relates the faction of all nuclei that are dSPNs (horizontal axis) to the fraction of all nuclei that are iSPNs (vertical axis), with a dashed line 1314 indicating equivalent values between the dSPN and iSPN fractions. A majority of the data points are below the dashed line 1314, indicating that the SPNs are more likely to be dSPNs in pwHD.

SPNs can also be categorized based on spatial locations to striosomes (patches) or the extrastriosomal matrix. For example, a fourth plot 1316 relates the faction of all nuclei that are matrix SPNs (horizontal axis) to the fraction of all nuclei that are patch SPNs (vertical axis), with a dashed line 1318 indicating equivalent values between the matrix SPN and patch SPN fractions. Striosomal (patch) SPNs were a reduced fraction of all SPNs in persons with HD, as indicated by a majority of the data points being below the line 1314, suggesting that patch SPNs were, on average, vulnerable earlier than extrastriosomal (matrix) SPNs. This is consistent with neuroanatomical measurements. Since striosomal (patch) SPNs receive inputs from cognitive and limbic structures (such as the amygdala, anterior cingulate gyrus and orbitofrontal cortex), whereas extrastriosomal SPNs receive more sensory and motor information, the earlier vulnerability of striosomal SPNs might help explain HD's early cognitive and psychiatric symptoms, which often precede motor symptoms but are less definitive diagnostically.

Loss of SPNs could in principle be accompanied by changes in the relative representation of cells of other types. Consistent with stereological studies of brain tissue from HD patients, the loss of interneurons was not detected, although one particular subtype of interneurons, cholinergic interneurons, exhibited a potential relationship (nominal p=0.02) that could be confirmed in a larger cohort. Loss of SPNs was accompanied by a clear loss of polydendrocytes (oligodendrocyte precursor cells or OPCs), which declined in numbers as neurons did, suggesting that numbers of OPCs may be regulated by numbers of neurons. When controlling for the loss of SPNs and polydendrocytes, the relative abundances of other cell types exhibited only modest changes (on average) as HD progressed.

### Expression of HTT in Relation to Neuronal Loss

A longstanding hypothesis for HD pathology invokes continuous damage from lifelong exposure of cells to a toxic mutant HTT (mHTT) protein. Based on this hypothesis, the primary strategy of pre-clinical therapeutic approaches to date involves reducing HTT expression, for example via antisense oligonucleotides. A better understanding of HTT expression levels and their relationship to the vulnerability of cell types, SPN subtypes, and individual persons was thus sought.

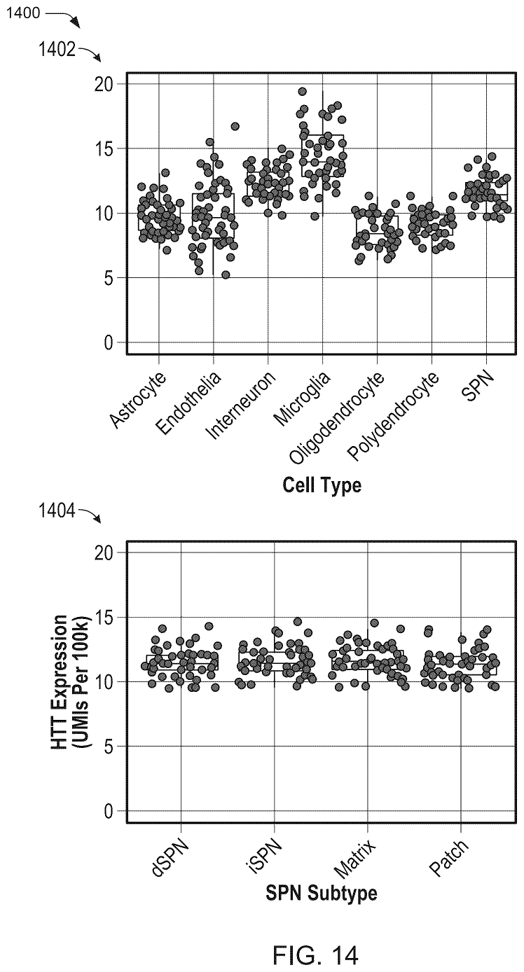

FIG. 14 shows an example 1400 comparing HTT expression. A first plot 1402 depicts the normalized expression of HTT (vertical axis) for different labeled subtypes (horizontal axis), and a second plot 1404 depicts the HTT expression level (vertical axis) for different labeled SPN subtypes (horizontal axis).

The first plot 1402 shows that quantitative biallelic expression levels of HTT, as a fraction of all mRNA transcripts, were slightly lower in SPNs than in interneurons, and only modestly higher in SPNs than in glia. Among SPNs, the second plot 1404 shows no evidence that the relative vulnerability of iSPNs (relative to dSPNs) or patch SPNs (relative to matrix SPNs) is reflected differences in HTT expression levels. HTT expression levels in dSPNs and iSPNs were indistinguishable (p=0.56, paired t-test). Striosomal (patch) SPNs (which are more vulnerable than matrix SPNs) exhibited a nominally lower HTT expression level than matrix SPNs did (p=0.01, paired t-test) HTT expression levels also exhibited inter-individual variation, but accelerated SPN loss (relative to CAP score) did not associate with higher HTT expression.

In every caudate cell type, thousands of genes were differentially expressed (on average) between persons with HD and unaffected individuals. This broadly altered gene expression potentially reflected the profound consequences of HD, which causes atrophy of the entire caudate, death of its principal neuronal population, and greatly changed life circumstances. Indeed, almost all such changes also associated (in an HD-cases-only analysis) with the extent of earlier SPN loss.

In summary, comparisons of cell types, SPN subtypes, and persons offered no support for the conventional “lifetime mHTT exposure” hypothesis, though they are not on their own a refutation of that hypothesis.

### Measuring Somatic CAG Expansion Alongside RNA Expression

A conventional approach to descriptive functional genomics in human disease involves comparing gene-expression data between cases and controls to arrive at a list of “differentially expressed genes” (DEGs). Single-cell analysis now allows DEGs to be identified for each cell type. However, even when applying a conservative statistical approach to identifying differentially expressed genes, every caudate cell type—including all types of neurons, glia, and vascular cells—exhibited thousands of DEGs whose expression levels differed (on average) between cases and controls.

This broadly altered gene expression in every cell type potentially reflects the profound consequences of HD, which causes atrophy of the entire caudate (reduced in HD to a small fraction of its normal size), neuronal death, and greatly changed life circumstances (such as paralysis). Such changes are likely to affect the biology of every cell type. For example, it was found that, across donors, the expression levels of DEGs correlated most strongly with the fraction of SPNs that a donor had already lost, a relationship that was true of SPN gene expression as well as gene expression in other cell types.

Because HD is caused by the DNA repeat in HTT, with longer repeats resulting in earlier onset, and because this DNA repeat exhibits mosaicism that could enable a biologically informative comparison of individual cells (sampled from the same donor) to one another, CAG-length-dependent cell-autonomous (inherent) gene expression changes that could be associated with a cell's own somatic expansion rather than with overall caudate atrophy was investigated according to the techniques described herein.

The CAG repeats of HTT transcripts were sequenced alongside genome-wide RNA expression in the same cells. The HTT CAG repeat is in the first exon of HTT, a gene that gives rise to a 165-kilobase (kb) pre-mRNA transcript and a 13-kb mature mRNA. The presence of the CAG repeat in exon 1 of HTT means that its length can be measured from mRNA transcripts of HTT. However, in using conventional techniques to prepare libraries by standard snRNA-seq methods, fewer than 0.001% of nuclei had an ascertained HTT transcript for which snRNA-seq sequencing reads touched both sides of the CAG repeat (e.g., for which the library potentially contained an informative molecule).

Accordingly, the techniques described herein, such as the workflow 200 of FIGS. 2A and 2B, creates molecular libraries from the same set of nuclei: one library samples genome-wide RNA expression (e.g., the transcriptome library 228), and another library specifically samples the 5′ region of HTT transcripts (e.g., the amplified short target cDNA library 258 and the amplified long target cDNA library 260 in combination). The presence of the cell barcodes 128, shared between the two libraries, allows each CAG-length measurement (e.g., the consensus repeat lengths 148 of FIG. 1) to be matched to the gene expression profile of the cell from which it is derived, and thus to the identity and biological state of that cell. Creation of these HTT-CAG libraries include the use of HTT-targeting primers at multiple steps, including the spike-in primers 220 used in the transcriptome amplification reaction 214 and the gene-specific primers 232 used in the targeted amplification reaction 230; HTT-targeted amplification and purification (e.g., the targeted amplification reaction 230 and the target purification 244); steps to preserve long molecules throughout library preparation (e.g., via the size separation 238); the calibration of amplification conditions to prevent the emergence of chimeric molecules during amplification; and analysis by sequencing (e.g., long read sequencing by the DNA sequencer 108).

The techniques described herein also include computational approaches to analyze the sequencing data 122 produced via the workflow 200. In the HTT-CAG libraries created by this approach, each individual HTT transcript (defined by a single UMI 130, for instance) is interrogated by very many sequencing reads of the sequencing data 122. Challenges arise from the fact that amplification routinely introduces artifactual repeat-length variation, chimeric molecules, and a quantitative bias toward shorter over longer molecules. However, reads with the same cell barcode 128 and UMI 130 may exhibit an informative consensus on the CAG length of the HTT transcript (e.g., the consensus repeat length 148). By utilizing the repeat length alignment module 136 and the repeat length analysis module 150 described with respect to FIG. 1, it is possible to detect and neutralize most distorting amplification reaction effects by calculating a consensus CAG length among all of the reads derived from the same HTT transcript. The use of the UMIs 130 is also helpful for computationally overcoming the PCR-biased over-amplification of short molecules compared with long molecules, as the UMIs 130 combined with the cell barcodes 128 enable each transcript to be counted exactly once.

To evaluate the accuracy of these single-cell CAG-length measurements, nuclei for which multiple measurements had been made from distinct mRNA transcripts (e.g., having different UMIs 130 and the same cell barcode 128) were assessed.

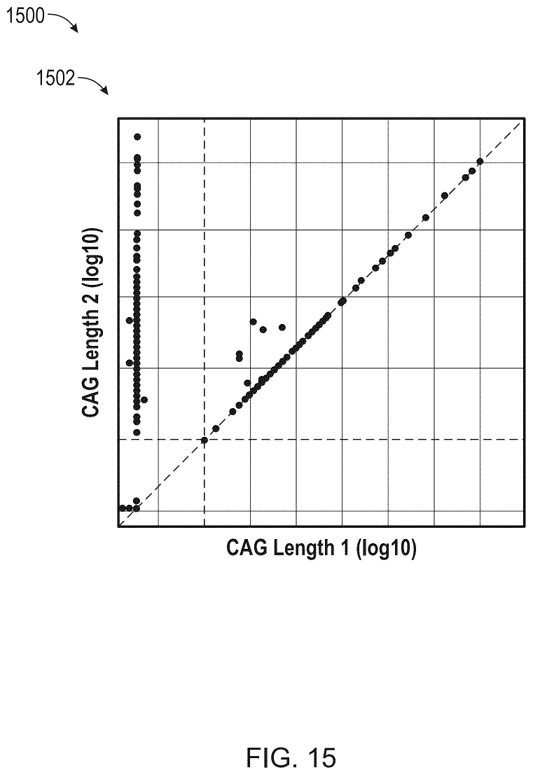

FIG. 15 shows an example 1500 of a plot 1502 of CAG measurement correlations. The horizontal axis of the plot 1502 represents a CAG measurement length of a first transcript (e.g., CAG length 1), and the vertical axis of the plot 1502 represents a CAG measurement length of a second transcript (e.g., CAG length 2). The plot 1502 shows concordance between pairs of measurements of CAG repeat lengths from different HTT RNA transcripts (with different UMIs 130) in the same nucleus (same cell barcode 128). For each such measurement-pair, the longer of the two CAG-repeat measurements is shown on the vertical axis. Horizontal and vertical lines demarcate three apparent cases: cases in both transcripts are from the HD-causing allele (upper right); cases in which both transcripts are from the normal allele (lower left); and cases in which the two transcripts are from distinct alleles (upper left). The nuclei in which both measurements are from the HD-causing allele (upper right) make it possible to measure the precision and error rate of this approach.

Among the subset of these measurements (from pwHD) for which both measurements appeared to arise from the HD repeat-expanded allele, measurements were highly correlated (with other measurements from the same cell) across a wide CAG-length range, with most of measurement-pairs agreeing, as shown by a generally linear trend in the datapoints from the lower left of the plot 1502 to the upper right of the plot 1502. For example, When CAG-repeat length could be measured on multiple HTT transcripts (with distinct UMIs 130) from the same allele (short or HD-causing) in the same nucleus, these measurements agreed.

These single-cell CAG-length measurements, relative to the CAG-length distributions yielded by bulk or sorted-cell approaches, tended to exhibit many more molecules with long DNA-repeat expansions. These included many nuclei with repeat expansions of 200-1000 repeats.

All pwHD exhibited evidence of somatic expansion; expansion in these donors always appeared to involve the inherited HD allele (initially greater than 35 CAGs) but not the other allele (stably less than 35 CAGs). In control donors, who had inherited two alleles with less than 35 CAGs, neither allele exhibited evidence of somatic expansion. This is consistent with earlier evidence from bulk analyses of brain tissue and suggests that the susceptibility of an HTT allele to somatic expansion is regulated in cis by the CAG length it has already attained, with 36 CAGs as a potential threshold for somatic expansion.

### Long Somatic CAG Repeat Expansions are Specific to SPNs.

Obtaining genome-wide RNA expression data and HTT CAG length in thousands of the same individual cells made it possible to characterize the cell-type specificity of somatic CAG expansion.

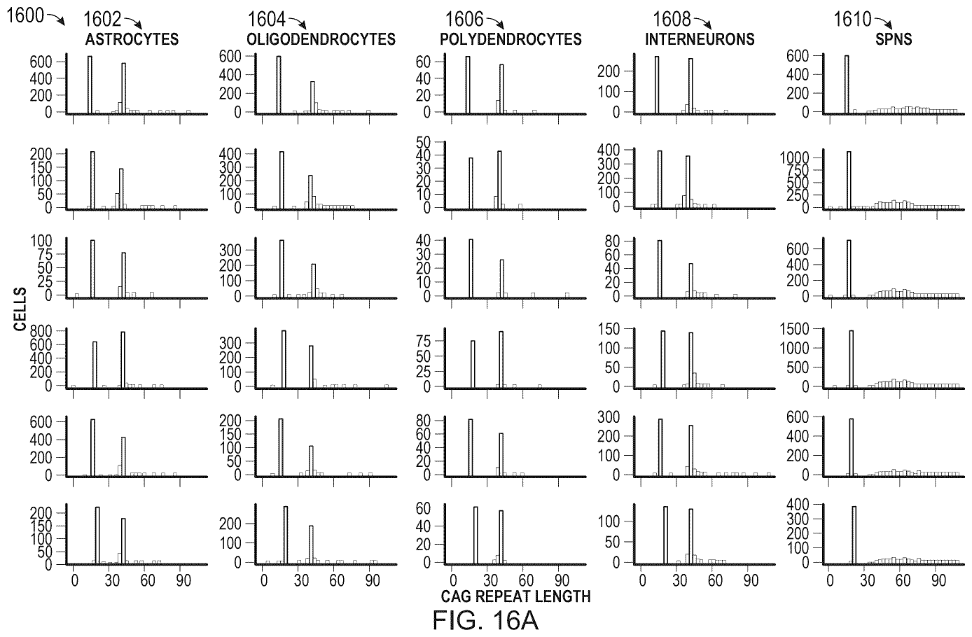

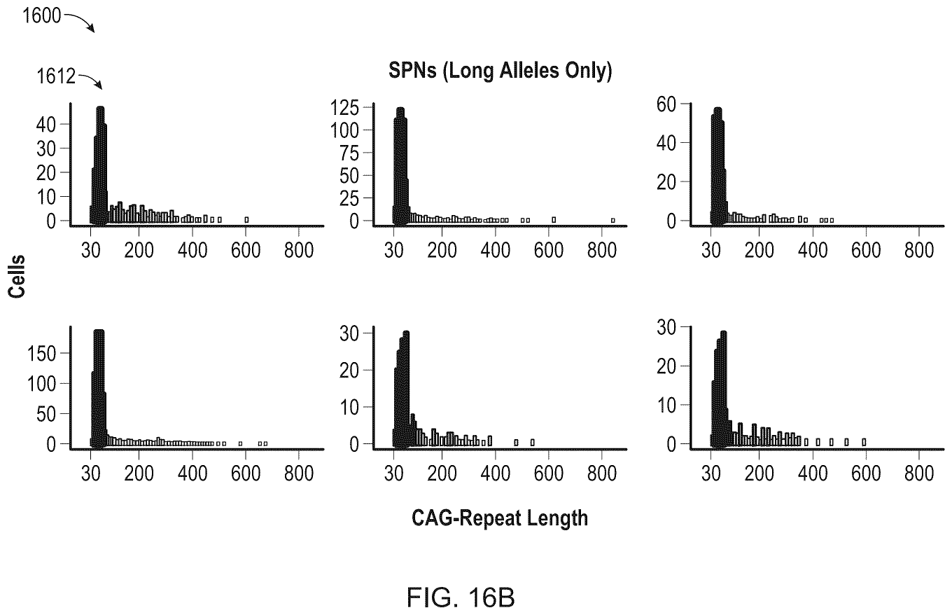

FIGS. 16A and 16B show an example 1600 of cell-type specificity of the CAG repeat length in HD. The example 1600 includes, in FIG. 16A, a plurality of plots, with each plot relating CAG repeat length (horizontal axis) to a number of cells (vertical axis). Each row of the plurality of plots of FIG. 16A represents data collect for a different donor, and each column of the plurality of plots represents a different cell type. For example, astrocyte CAG repeat length plots 1602 are shown in a first column, oligodendrocyte CAG repeat length plots 1604 are shown in a second column, polydendrocyte CAG repeat length plots 1606 are shown in a third column, interneuron CAG repeat length plots 1608 are shown in a fourth column, and SPN CAG repeat length plots 1610 are shown in a fifth column.

Referring to FIG. 16A, as shown the example 1600, the HTT CAG repeat exhibits profoundly different length distributions in different cell types. Astrocytes (e.g., the astrocyte CAG repeat length plots 1602), oligodendrocytes (e.g., the oligodendrocyte CAG repeat length plots 1604), microglia, endothelial cells, and interneurons (e.g., the interneuron CAG repeat length plots 1608) exhibit modest CAG repeat instability, with almost all cells exhibiting a distribution of CAG lengths within a few units of the inherited length. However, SPNs exhibit extensive somatic expansion of the HD-causing allele (e.g., the SPN CAG repeat length plots 1610). Somatic expansion appears to be allele-specific, as the somatic expansion is exhibited by the HD-causing allele but not the other inherited allele in each pwHD.

The distinction between SPNs and striatal neurons is particularly notable because they are inhibitory (GABAergic) neurons that arise from a shared developmental lineage. Among interneurons, cholinergic interneurons exhibit more expansion than other interneurons, though far less than SPNs.

FIG. 16B shows a plurality of plots 1612 of distributions of CAG repeat length measurements in SPNs, specifically showing the long (HD-causing) allele and the much-wider range of CAG repeat lengths the SPNs attain. As shown in the plurality of plots 1612, the distributions of SPN CAG repeat lengths in persons with clinically apparent HD exhibit a characteristic shape that visually resembles the profile of an armadillo, with a large body and a long, slowly tapering tail. The bulk of the distribution (the armadillo's “body” on the left of the distribution) reflect substantial expansion in almost all SPNs, with 95-98% of each donor's SPNs expanding beyond the inherited (germline) length and reaching a median CAG repeat length of 60-73 CAGs (20-31 CAGs longer than the same donors' germline HTT alleles of 40-43 CAGs). Thus, almost all SPNs appeared to have experienced very many expansion events across each donor's lifespan.

The second feature (the armadillo's “tail” extending toward the right of the distribution) includes a prominent minority of SPNs with far longer expansions (e.g., 100 to 500 or more CAGs). This long, prominent, right tail that commences at about 100 CAGs and tapers slowly across a wide range (e.g., 100 to 500 or more CAGs). It is contemplated that these two parts of the distribution—the “body” (e.g., 36-100 repeat units) and the “tail” (e.g., 100-500 or more repeat units)—may reflect two distinct phases of somatic expansion (phase A and phase B), with the rate of expansion greatly increasing as the repeat expands beyond about 100 CAGs.

Thus, the cell-type-specific vulnerability of SPNs, and the relative quantitative vulnerabilities of SPN subtypes, appear to correspond to rates of somatic CAG expansion in these cell types and subtypes.

The detection of many SPNs with long repeats (100-1000 CAGs) contrasted with earlier human HD studies, many of which detect repeat expansion only in the 35-100 range. The use of the UMIs 130 according to the techniques described herein enables these long CAG repeat expansions to be detected. For example, the UMIs 130 enable analytical correction for the tendency of PCR to amplify smaller molecules exponentially more efficiently than larger ones.

Expansion to 150 CAGs without Cell-Autonomous Consequence

To recognize how HTT CAG repeat length directly affects gene expression in the SPN in which it has expanded, “allelic series” of SPNs were identified, each including 467 to 2,337 SPNs from within the caudate of an individual person with HD, collectively spanning 35 to 842 CAGs. By performing each analytical comparison within-person rather than across people, the profound non-cell-autonomous effects of each donor's disease state was controlled for.

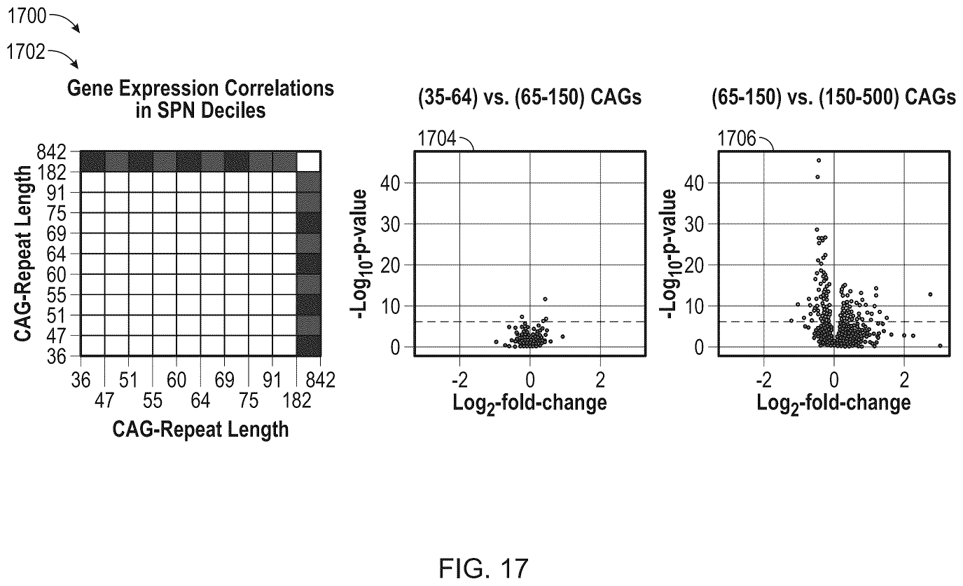

FIG. 17 shows an example 1700 of comparing HTT CAG repeat length and gene expression in SPNs. The example 1700 includes a plot 1702 depicting a magnitude of gene expression differences (one minus the correlation coefficient) when comparing sets of SPNs (from the same tissue sample) grouped into deciles based on the CAG repeat length of the HD-causing HTT allele. Black indicates maximal difference observed in a comparison, while unfilled boxes indicate no difference. As such, darker pixels (e.g., closer to black) indicate more difference than lighter pixels.

The example 1700 further includes a first expression plot 1704 comparing gene expression in SPNs with 35-64 CAG repeat lengths with those having 56-150 CAG repeat lengths and a second expression plot 1706 comparing gene expression in SPNs with 65-150 CAG repeat lengths with those having greater than 150 CAG repeat lengths. Surprisingly, the first expression plot 1704 shows that these SPN populations (when sampled from the same donor) exhibited no apparent differences in RNA expression to 150 CAGs. In contrast, the second expression plot 1706 shows that SPNs with extremely long expansions (e.g., 150 or more CAG repeat units) differed profoundly in gene expression in comparison to nearby SPNs with more modest CAG repeat lengths (65-150 CAGs).

All of the probands with clinically apparent HD exhibited a similar pattern, with no significant CAG length-associated changes among groups of SPNs with up to 150 CAG repeat lengths, but profound differences in comparisons that involved SPNs with repeat expansions longer than 150 CAGs.

Gene Expression Changes with CAG Repeat Expansion Beyond 150 CAGs

The specific gene expression changes associated with long somatic CAG repeat expansions (e.g., greater than 150 CAG repeat units) are almost identical from person to person, indicating that HTT-CAG repeat expansion beyond 150 CAG repeat lengths changes SPN gene expression in a way that is both consistent across individual SPNs in the same person and consistent across different persons with HD. This contrasts strongly with the “differentially expressed genes” previously identified in comparing HD cases to controls; these genes' expression levels tended only to differ on average (and not in each individual case, and to an extent that was predicted by the donor's earlier SPN loss.) These specific changes may be the direct, cell-autonomous effects of repeat expansion (controlling for conditions that may be more specific to a donor's disease stage, age, and health history).

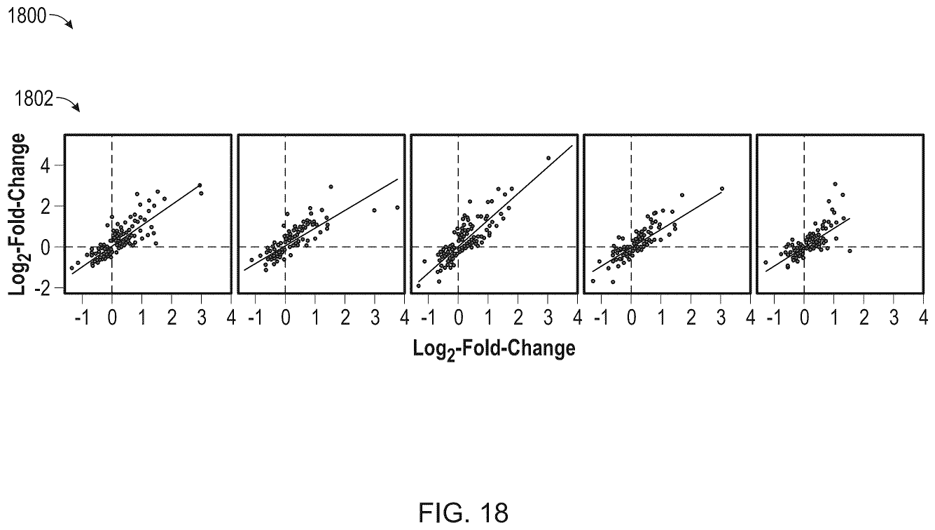

FIG. 18 shows an example 1800 of a plurality of plots 1802 demonstrating consistency of long repeat expansion-associated gene expression changes across individual persons with HD. Each panel of the plurality of plots 1802 is a pairwise comparison of SPN data from two persons with HD (e.g., a first donor on the horizontal axis and a second donor on the vertical axis), in which the values plotted are the log 2-fold-changes in gene expression when comparing (within-tissue) SPNs with greater than 150 CAG repeat lengths to SPNs with less than 150 CAG repeat lengths. Genes whose expression levels change significantly with repeat expansion in at least one of the donors are shown.

Further evidence that CAG length-driven gene expression changes arise at long CAG repeat lengths may be presented by regression analysis (negative binomial regression), in which the expression level of each gene may be fit to a combination of donor effects, SPN-subtype effects, and CAG repeat-length effects. Some genes exhibit expression levels that correlate with CAG repeat length by systematically showing stronger relationships to a “hinge function” (e.g., in which CAG repeat length has no effect until reaching 150 units) than to a naive “linear function” (e.g., in which CAG repeat length affects gene expression across its full range). For example, an analysis may identify no substantial set of “dissenting” genes that associate more strongly with the naive model. The model with a hinge at 150 may also out-perform models with hinges at 120, 135, 165, or 180 repeats.

The nearly identical nature of these long expansion-associated gene expression changes from patient to patient may enable the use of the donors' data together to identify genes whose expression levels are affected by CAG repeat length. Notably, these genes may exhibit two kinds of relationships to CAG repeat length. A first set of genes may exhibit continuous change in expression levels as the CAG repeat further expands beyond 150 CAGs. A second set of genes may exhibit discrete and dramatic changes in a specific subset of these SPNs with still longer CAG repeat expansions (e.g., greater than 250 CAGs), as further elaborated below.

The measurements of HTT expression may exhibit no correlation with CAG repeat length, although this does not preclude the possibility that post-transcriptional processing of HTT transcripts changes with CAG repeat expansion.

Continuous Changes with Expansion Beyond 150 Units

It was found that more than two hundred genes exhibit incipient and escalating gene expression distortion to the extent the CAG repeat had beyond 150 units. This may be referred to as phase C (continuous change), and to the affected genes as C− (downregulated) and C+ (upregulated) genes.

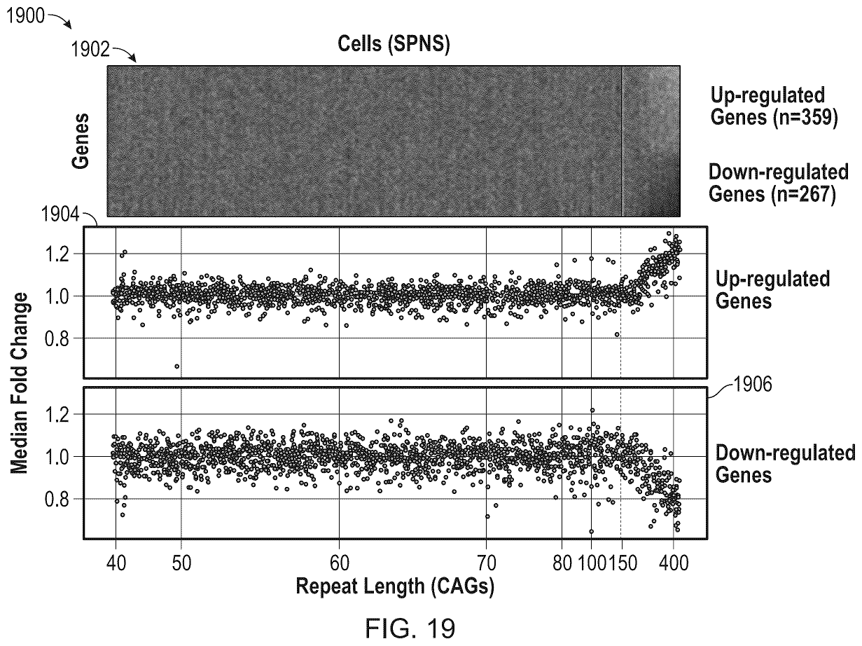

FIG. 19 shows an example 1900 of continuously escalating gene expression distortion beyond 150 CAG repeat lengths. The example 1900 includes a heat map 1902 showing upregulated genes (relative to the average SPN in that donor) as lighter pixels and downregulated genes (relative to the average SPN in that donor) as darker pixels. A specific donor's individual SPNs are ordered from left to right by their CAG repeat length (thus corresponding to the columns of the heat map 1902). Each row shows expression data for a specific gene in each of these SPNs. The genes shown are those found to change in expression concurrently with further repeat expansion beyond 150 units. For example, the heat map 1902 shows that the upregulated genes and down regulated genes become increasingly clustered at CAG repeat lengths greater than 150. This is also demonstrated in a first median fold change plot 1904 quantifying the upregulated genes and a second median fold change plot 1906 quantifying the downregulated genes.

Repeat length-associated expression changes were observed to be almost undetectable at 150-180 repeat lengths, but analyses that drew upon all of the genes together indicated that these changes had commenced at approximately 150 repeat lengths.

This pattern indicating the absence of any clear cell-autonomous biological change before 150 CAG units followed by progressively escalating change with expansion beyond 150 was shared across each of the individual persons with HD that were analyzed. It was also shared by direct and indirect SPNs, and by patch and matrix SPNs. Although individual persons with HD varied in the fraction of their SPNs that had attained long repeats at the end of life, all appeared to share the high threshold (of about 150 CAGs) at which these same gene-expression changes had commenced in individual SPNs.

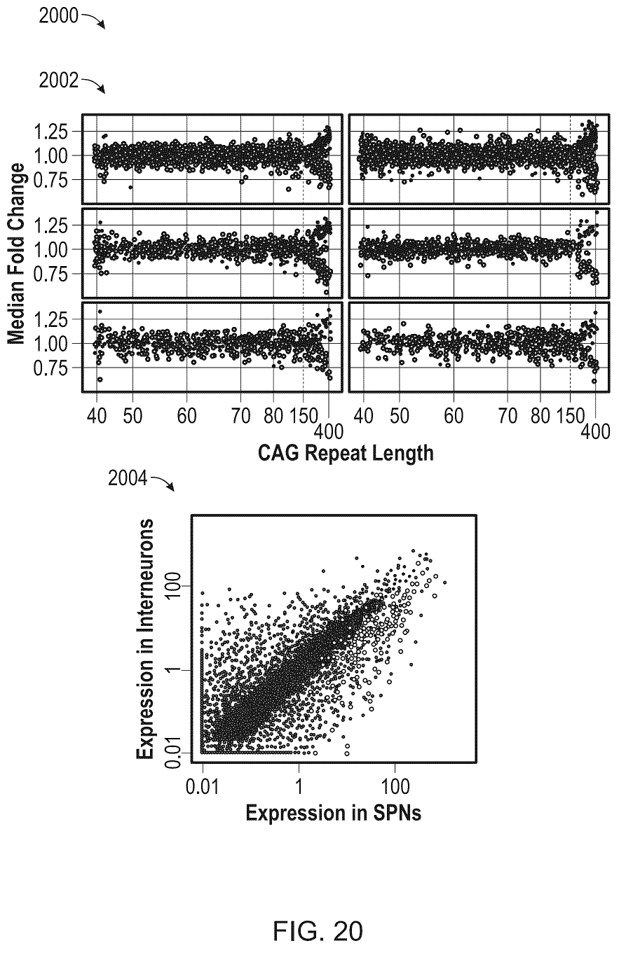

FIG. 20 shows an example 2000 of median fold change plots quantifying upregulated and downregulated genes for a plurality of individual persons with HD. The example 2000 includes a plurality of plots 2002, each plot of the plurality of plots 2002 indicating the median fold change for an individual person with HD. On each of the plurality of plots 2002, a specific person's individual SPNs are ordered from left to right by their CAG repeat length. Each of the plurality of plots 2002 shows progressively escalating change in gene expression after 150 CAG repeat lengths.

The example 2000 further includes a plot 2004 of gene expression features of SPN identity and phase-C changes. Expression in SPNs is indicated on the horizontal axis, and expression in interneurons is indicated on the vertical axis. The genes whose expression levels decline in SPNs as their HTT CAG repeat expands further beyond 150 units (C-genes) tend to be genes that are more strongly expressed in SPNs than in nearby striatal interneurons (e.g., the points are lower and further right).

To try to better understand the nature of the biological changes elicited by long (greater than 150 CAG repeat length) somatic expansions, biological patterns shared by the genes mis-regulated in SPNs with long repeat expansions were investigated. A profound pattern related to SPN identity was identified: the declining (C-) genes were among the most strongly expressed genes in SPNs and were systematically genes whose normal expression levels in SPNs distinguished SPNs from other types of inhibitory neurons. These included PDE10A, PPP2R2B, PPP3CA, PHACTR1, RYR3, and more than 100 other genes that normal SPNs express more strongly than striatal interneurons. This suggests that a core biological change in phase C involves the steady, quantitative erosion of features that distinguish normal SPNs from other kinds of inhibitory neurons.

Although the primary biological property shared by the genes that declined in expression during phase C was the way their expression distinguished normal SPNs from other cell types, many of these genes also have known physiological functions. For example, genes encoding the potassium channel subunits KCND2, KNCQ5, KCNJ10, KCNJ16, and KCNMA1 all declined in expression during phase C, a change that might affect SPN physiology.

HTT expression itself did not associate with an SPN's own CAG repeat length, although this does not preclude altered post-transcriptional processing that single nucleus RNA-seq does not measure. HTT expression was slightly lower in the donors who had passed away with the greatest caudate atrophy (>90% SPN loss), but this decline appeared to be a sequela of extreme atrophy, as it did not associate with CAG-repeat length within any donor.

Although the relationship of phase C changes to a cell's own CAG repeat length were strong and clear, such changes appear to have been hard to recognize in earlier human brain studies because they arise asynchronously in sparse individual SPNs. Earlier studies have focused on changes that analyses suggested were downstream consequences of SPN loss, as they were experienced equally by all surviving SPNs and their magnitude associated with a donor's earlier caudate atrophy.

Stronger alignment was found between the present findings and analyses of a specific HD mouse model (Q175), which begins life with a CAG repeat tract of about 175 CAGs in all cells. In such mice, SPNs, interneurons and glia all exhibit reduced expression of genes that distinguish them from one another. The presence of this long (175 CAG) repeat in all cell types (rather than just SPNs) may also explain the diverse cell-type-specific pathologies, including vascular and endothelial pathologies, exhibited by Q175 mice.

De-Repression Crisis with Still-Longer CAG Repeats

The above trajectory of continuously escalating gene-expression distortion with further repeat expansion beyond 150 units generally involved genes that are strongly expressed by normal SPNs. A distinct set of genes that are normally repressed in SPNs also exhibited repeat-length-dependent change, but with a very different pattern. These genes remained repressed even in most SPNs with long (e.g., greater than 150 CAG repeat length) expansions, but became de-repressed in a subset of these SPNs in which the phase C changes had progressed to the greatest degree. In the cells in which de-repression had occurred, it tended to involve very many genes at once. This state is referred to herein as a “de-repression crisis” (phase D).

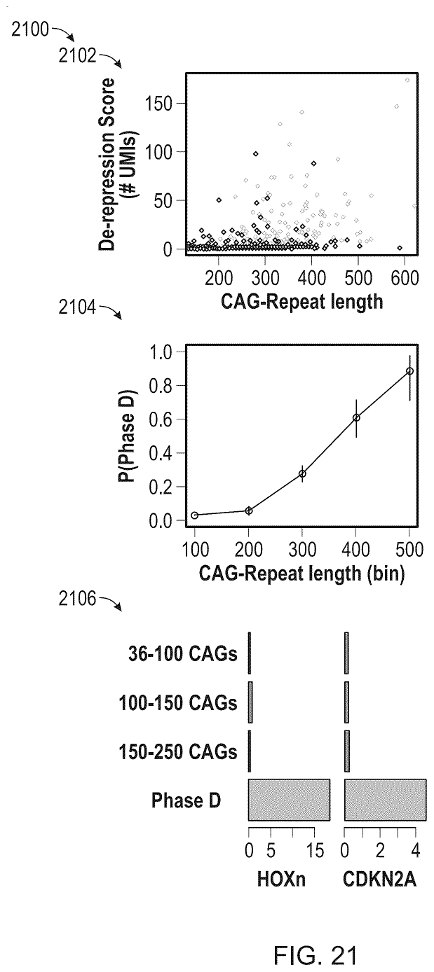

FIG. 21 shows an example 2100 of de-repression in genes having long CAG repeat expansions. The example 2100 includes a first plot 2102 comparing CAG repeat length (horizontal axis) to a de-repression score based on a number of UMIs identified and a second plot 2104 comparing the comparing CAG repeat length (horizontal axis) to a p value of the de-repression state (vertical axis). The example 2100 further includes a bar graph 2106 showing the expression of HOX cluster genes (left) and CDK2NA (right) in SPNs of person with HD, with horizontal axis units referring to UMIs per 100,000. CAG repeat length ranges are shown on the vertical axis.

In the cells in which de-repression occurred, it tended to involve all or very many of these genes at once: while 75% of SPNs with long expansions (>150) continued to show almost no expression (across these cells: 0-3 UMIs, median 0, of these 89 genes together), the other 25% exhibited widespread expression of these same genes (4-129 UMIs, median 10). The de-repression of these genes thus appeared to behave as a discrete event in which very many of the genes had become de-repressed within a short period of time.

The likelihood that an SPN was in a de-repression crisis (phase D) exhibited a strong relationship to its CAG repeat length, and generally was associated with still-longer expansions than identity-softening (phase C) was. De-repression was rare (<4%) even among SPNs with 150-250 CAG repeats, but it became very common (>50%) in SPNs with 350 or more CAGs. In nuclei in which these phase D changes were detected, the magnitude of these changes bore little relationship to CAG-repeat length, as shown in the first plot 2102. This pattern is distinct from the phase C changes, which were well-predicted by an SPN's CAG-repeat length at the time of analysis. This is interpreted to mean that, while phase C changes proceed on a time scale similar to that of fast CAG-repeat expansion (beyond 150 CAGs), phase D changes proceed with far-faster kinetics once underway.

The 173 genes found to be de-repressed in phase D had a distinct set of biological features in common. These included the large clusters of genes at the HOXA, HOXB, HOXC, and HOXD loci, as well as noncoding RNAs (HOTAIR, HOTTIP, HOTAIRMI) at these same genomic loci. These genes are involved in cell specification in the brain and other organs and are normally expressed during early embryonic development but not in adult neurons. The de-repressed genes also included transcription factor genes at dozens of loci across the genome that are normally expressed in other neural cell types but not in SPNs (including FOXD1, IRX3, LHX6, LHX9, NEUROD2, ONECUT1, POU4F2, SHOX2, SIX1, TCF4, TBX5, TLX2, ZIC1, ZIC4).

The widescale de-repression of transcription factor genes might lead to expression of genes normally expressed in other neural cell types. Indeed, phase D SPNs expressed many genes that are normally expressed in interneurons (CALB2, SST, KCNC2), in glutamatergic (excitatory) neurons (SLC17A6, SLC17A7, SLC6A5), in astrocytes (SLCIA2), in oligodendrocyte progenitor cells (VCAN), or in oligodendrocytes (MBP). These changes suggested that SPNs in phase D were losing negative as well as positive features of cell identity.

As shown in the bar graph 2106, two of the most strongly de-repressed genes were CDKN2A and CDKN2B, which encode proteins (p16(INK4a) and p15(INK4b)) that promote senescence and apoptosis in many cellular contexts. Ectopic expression of Cdkn2a is toxic to neurons. De-repression of CDKN2A and CDKN2B in phase D SPNs may be an imminent cause of their death. Inactivation of the Polycomb Repressor Complex 2 (PRC2) in adult mice also causes de-repression of Hox genes, other transcription factors, and Cdkn2a and Cdkn2b, leading within months to SPN loss, motor function decline and lethality.

Differentially expressed genes in neuron with long CAG repeat expansions are provided below in Table 10.

TABLE 10

| Genes downregulated in neurons with | | | Genes upregulated in neurons with | | | |
|---|---|---|---|---|---|---|
| long CAG repeat expansions | | | long CAG repeat expansions | | | |
| ABAT | EML1 | PDE10A | AAK1 | GRIN1 | WDR60 | HOXC4 |
| AC022387.1 | EPB41L2 | PDZD2 | AC010615.4 | HDAC8 | WNK2 | HOXC6 |
| AC022613.3 | EPHX4 | PHACTR1 | ADD3 | JAKM1P2 | XIST | HOXC8 |
| AC069437.1 | EYA1 | PLEKHA5 | AEBP2 | KCNQ2 | YWHAE | HOXC9 |
| AC073050.1 | FAM171A1 | PPP1R16B | AFDN | KIAA0586 | ZNF609 | HOXD-AS2 |
| AC078909.1 | FAT3 | PPP2R2B | AGAP1 | KIAA0825 | ZNF638 | HOXD10 |
| AC090124.2 | FOXG1 | PPP3CA | AKAP13 | KSR2 | ABCC9 | HOXD11 |
| AC093523.1 | FOXP1 | PRKCB | AL589740.1 | LSM14A | AC004080.2 | HOXD3 |
| AC109466.1 | FUT9 | PTPN5 | ANKRD11 | MAGI2 | AC008060.1 | HOXD8 |
| AC127164.1 | GABPA | RAP1GAP | ANKRD30BL | MAP1B | AC008060.4 | HOXD9 |
| ADAMTS19 | GNAL | RAPGEF5 | ARFGEF1 | MAPK10 | AC012531.1 | HSD17B11 |
| ADAMTS3 | GPR137C | RARB | ARHGAP26 | MAPT | AC016044.1 | HSPA1A |
| ADCY5 | GRIA3 | RERG | ARID4B | MED27 | AC021016.3 | HSPA1B |
| AKAP5 | GRM5 | RFX4 | ATRX | MINK1 | AC022201.2 | HTR7 |
| AL020994.3 | HRH2 | RIC8B | BDP1 | MIR100HG | AC023300.2 | IFIT3 |
| AL049830.3 | HS2ST1 | RNF144A | C9orf3 | MLLT10 | AC106733.1 | IRX5 |
| AL355472.3 | HTR2C | ROCK2 | CACNA1C | MPPED2 | AC139712.3 | LHX9 |
| ANKS1B | HTR4 | RYR2 | CACNA1D | MROH1 | ACSS1 | LINC00434 |
| ANO3 | IGSF11 | RYR3 | CAMK2A | MYCBP2 | AL162741.1 | LINC00682 |
| ARHGEF10 | INPP5A | SIPA1L1 | CAPN7 | N4BP2L2 | AL353052.1 | LINC01682 |
| ARMC9 | JPH1 | SLC24A2 | CCDC84 | NCOA1 | AL449106.1 | LINC01977 |
| ARPP19 | KCNA4 | SLC35F3 | CCDC88A | NRG1 | AP006248.3 | LINC02219 |
| ARPP21 | KCNAB1 | SLC47A1 | CCSER1 | NTRK2 | APOL6 | LINC02473 |
| ATL2 | KCND2 | SLC9A7 | CDC42BPA | NUP58 | CCDC140 | MAPKAPK2 |
| ATP2B1 | KCNQ5 | SORBS1 | CDKN2A | PCNX2 | ELAVL2 | NEUROD2 |
| B3GLCT | LAMP5 | SPOCK1 | CDKN2B | PNISR | FAM46A | NKX2-5 |
| BBS2 | LDLRAD4 | STARD4 | CHD9 | PRKCI | FGF5 | OR3A1 |
| C6orf141 | LINC01085 | STXBP5L | CHN2 | PSIP1 | FP671120.1 | OTP |
| CA12 | LINC01250 | SV2C | CSMD3 | RFX3-AS1 | HAND2 | PITX2 |
| CACNA1E | LINC01551 | TANC2 | CTIF | RTN3 | HIST1H4D | PNMA8C |
| CACNA2D3 | LINC02137 | TCAF1 | CUL3 | SGSM1 | HOTAIRM1 | POU4F2 |
| CAMK4 | LMO3 | TESC | DNM1L | SHANK2 | HOXA-AS3 | PRDM13 |
| CCDC6 | LPL | THSD4 | DYNC1I1 | SNHG14 | HOXA10 | PRKCQ-AS1 |
| CCDC88C | LRRC7 | TJP1 | EIF4G3 | SON | HOXA10-AS | RAB3B |
| CDC40 | MACROD2 | TLE1 | FAF1 | SRPK2 | HOXA11-AS | RAET1G |
| CDS1 | MALAT1 | TLE4 | FAM153B | SRRM2 | HOXA2 | SHOX2 |
| CELF2 | MAN1A1 | TMOD1 | FAM153C | SRSF11 | HOXA3 | SLC6A5 |
| CHSY3 | MAPK4 | TOMM70 | FAM222B | STOX2 | HOXA4 | SNTB1 |
| CIT | MBNL2 | UNC80 | FRMD4A | STRN3 | HOXA5 | SULT1C4 |
| CKB | MIR222HG | USP25 | FRMD4B | SYBU | HOXA6 | TBX5 |
| CNKSR2 | MOB3B | VAV3 | FTX | TAOK1 | HOXA7 | TBX5-AS1 |
| COCH | MTHFD1 | WASF1 | GABRG3 | TAPT1-AS1 | HOXB-AS1 | TP53INP1 |
| CRYBG3 | NETO2 | WFS1 | GIGYF2 | TARBP1 | HOXB-AS3 | TWIST1 |
| DAPK1 | NGEF | YPEL1 | GNB1 | TBCK | HOXB2 | ZIC4 |
| DCC | OSBPL8 | ZMAT4 | GPATCH8 | TENM4 | HOXB3 | |
| DGKB | PASK | ZNF83 | GPHN | TMEM232 | HOXB6 | |
| DGKI | PBX4 | ZSWIM6 | GPI | TMEM241 | HOXB7 | |
| DLGAP1 | PCP4 | | GRIA1 | TNRC6A | HOXC10 | |

SPN Loss Coincides with the Appearance of Long CAG Repeat SPNs

The above results suggested that the transcriptional changes in long-repeat (phase C and D) SPNs might closely precede their death. Though the same human tissue may not be observed at multiple time points using post-mortem tissue, comparisons across donors who passed away at different stages of caudate atrophy and SPN loss may enable comparisons. To do this, CAP scores were used as a measure of progression to bring donors with a variety of ages and inherited CAG repeat lengths into a common analysis. Analysis indicated that the rate of SPN loss (as a function of CAP score) tracked closely the fraction of donors' SPNs that were in phase C or D.

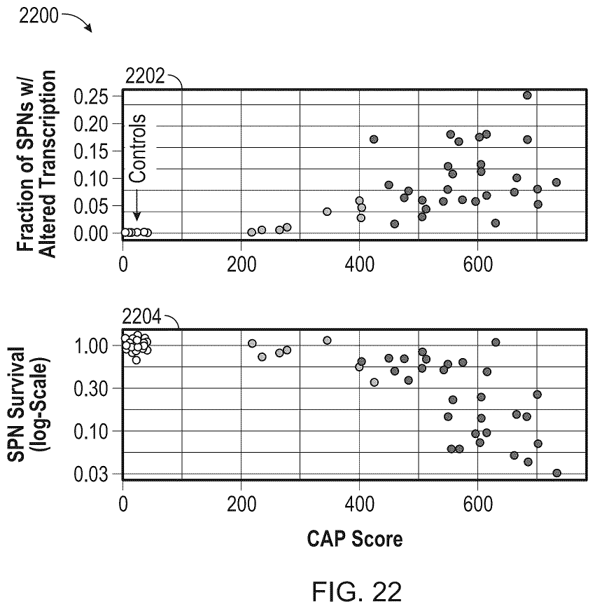

FIG. 22 shows an example 2200 analysis of transcriptional changes in relation to CAP score. The example 2200 includes a first plot 2202 comparing the CAP score (horizontal axis) to a fraction of SPNs having altered transcription (vertical axis) and a second plot 2204 comparing the CAP score (horizontal axis) to SPN survival (vertical axis). Across HD onset and progression (as indexed by CAP score), the rate of SPN loss (e.g., the slope of the decline in SPN abundance of the second plot 2204) reflected closely the fraction of donors' SPNs that were in phase C or D at these same HD stages (e.g., the first plot 2202). This is consistent with the idea that neurons experience phases C and D shortly before dying. This may also explain why phase C and D SPNs are a small fraction of all SPNs throughout HD.

### A Model for SPN Pathology in HD

Based on the results from the examples described with respect to FIGS. 12-22, a model for the pathology of SPNs in HD is proposed that involves four sequential phases, each driven by the length of the CAG repeat in a cell's own HD-causing allele.

In the first phase (phase A, when a neuron has 36 to about 80 repeat units), an SPN undergoes decades of slow repeat expansion. It is estimated that an SPN may take a first length of time to expand from 40 to 60 repeats, then a second length of time to expand from 60 to 80 repeats. This expansion appears to be biologically quiet in the sense that cell-autonomous effects of the CAG repeat upon the cell's own gene expression are not detected. The long time a cell spends in phase A helps explain the disease's late onset and the effect of inherited CAG repeat length on age at onset; it may also explain why so many of the common genetic modifiers of HD age-at-onset involve variation which plausibly affects somatic expansion. Phase A could be compared to a slowly and capriciously ticking DNA clock.

As a neuron enters the second phase (phase B, 80 to 150 repeat units), the rate of expansion greatly accelerates. Having taken decades to expand to 80 repeats, a neuron may now expand to 150 in just a few years. This acceleration accounts for the observation that a donor can simultaneously have modest expansion (36-80 repeats) in the great majority of neurons, and extremely long expansions (100-500+ repeats) in others, e.g., the long, slowly tapering tail of the armadillo-shaped distribution of SPN CAG repeat lengths shown in the plurality of plots 1612 of FIG. 16B. Still, as in phase A, the neuron's own gene expression does not appear to change under the influence of its own HTT CAG repeat. Phase B could be compared to a more rapidly, predictably ticking DNA clock.

As a neuron enters the third phase (phase C, 150+ repeat units), hundreds of genes begin to change in expression levels. These changes escalate as the repeat further expands, such as demonstrated in the example 1900 of FIG. 19 and the example 2000 of FIG. 20. This relationship could reflect an increasingly toxic HTT entity; alternatively, since the repeat at this stage is expanding quickly and predictably, it may reflect the number of weeks that a neuron has had a CAG repeat longer than the toxicity threshold.

In the fourth phase (phase D, generally observed in association with still-longer repeats, though with a less predictable relationship to repeat length than in phase C), SPNs appear to undergo a kind of de-repression crisis, expressing scores of genes that are normally silenced in adult neurons. Neurons in phase D also begin to express CDKN2A and CDKN2B, which encode well-established drivers of senescence and apoptosis. Such neurons are rare (<0.1% of nuclei) at early HD stages, but they become more abundant (0.5-2%) as HD progresses into periods of rapid SPN loss and caudate atrophy.

Finally, in phase E, a cell is eliminated. Such cells disappear from the CAG length and gene expression data, but the effects of their loss upon remaining cells are likely strong, for example, in the de-neuralization and atrophy of the caudate, and in a person's changed life circumstances, all of which seem likely to affect gene expression. The gene-expression changes in all cell types in HD (including those changes in SPNs that were uncorrelated to a cell's own CAG repeat length) were systematically correlated with a donor's earlier SPN loss.

Individual SPNs appear to progress through these phases at different times, an asynchrony which modeling suggests can be explained largely by the variable amounts of time that individual neurons take to traverse phase A. Phase A introduces this asynchrony because each neuron's expansion results from low-frequency stochastic length-change mutations (initially occurring less than once per year) and because each expansion event increases the likelihood of subsequent expansion events. As a result, individual SPNs progress from phase A to phases B-D at different times.

Though human HD patients with typical mid-life onset has been described with respect to FIGS. 12-22, this model could in principle also help understand the faster and more-synchronous pathology observed in widely used mouse models of HD (such as Q111, Q175, R6/1, and R6/2, all of which start life with more than 110 repeats), as well as the fast disease progression observed in rare, childhood-onset HD patients who have inherited alleles with more than 70 repeats (i.e., close to or beyond the end of phase A).

### Example 2: Computational Modeling of Repeat Expansion Dynamics

The above-described findings differ from conventional biological models of HD in which most or all SPNs endure a toxic mutant HTT simultaneously. Therefore, it was investigated whether a model of sequential SPN toxicity in which, at any one time, most SPNs have a biologically innocuous HTT whose repeat length is far below a high toxicity threshold could explain the loss of SPNs in HD.

To better appreciate the dynamic processes that might give rise to clinical observations and end-of-life biological measurements, repeat expansion dynamics over the human lifespan were computationally modeled, e.g., using the expansion dynamics modeling module 154 of FIG. 1. A goal of the computational modeling is to understand whether simple models based on an emerging understanding of DNA repeat expansion mechanisms would generate repeat length distributions and cell loss trajectories consistent with experimental results.

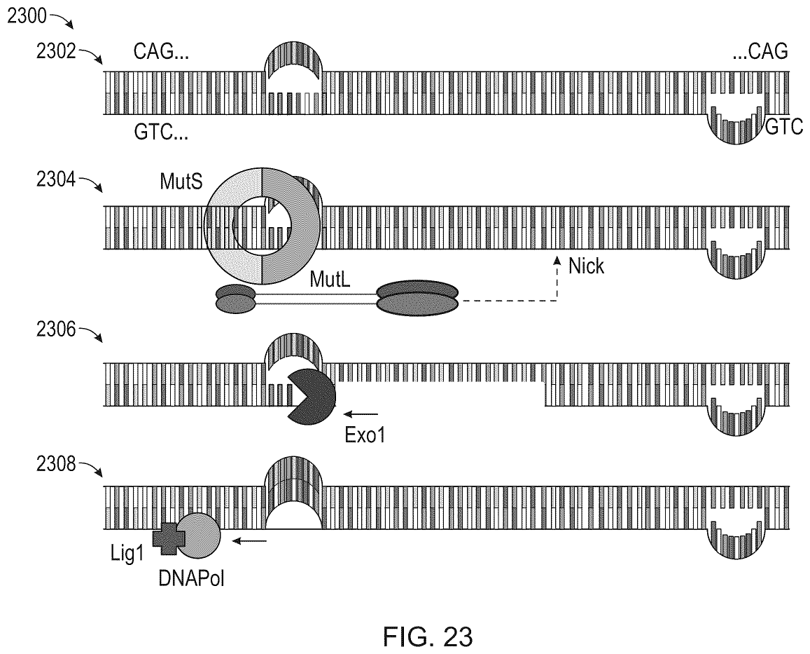

FIG. 23 shows an example schematic 2300 of a hypothesized model for post-mitotic repeat expansion. In post-mitotic cells, DNA repeat length-change mutations are thought to result from occasional strand misalignment (e.g., mispaired repeats) after transcription or transient helix destabilization. Mispaired repeats create extrahelical extrusions (“slip-out” structures), as shown in a first diagram 2302 of the example schematic 2300.

Small extrahelical extrusions are recognized by mismatch repair (MMR) complexes, which initiate repair pathways that involve nicking (shown in a second diagram 2304 of the example schematic 2300), excision (shown in a third diagram 2306 of the example schematic 2300), and resynthesis (shown in a fourth diagram 2308 of the example schematic 2300) of one of the two strands. If the two slip-out structures are farther apart than this excision distance, then resynthesis results in a length change mutation—an expansion or contraction, depending on which strand has been nicked and excised. Moreover, MMR complexes have a strand bias that leads to expansions more frequently than contractions. Experimental observations indicate that repeat expansion tends to occur in small increments.

Simulations (e.g., produced via the expansion dynamics modeling module 154 of FIG. 1) adhere to this emerging understanding. In at least one implementation, the modeling assumes that all SPNs initially had the same (germline) HTT allele, that length change mutations were stochastic expansions or contractions that generally changed the repeat length by a small number of CAG units, that the likelihood of mutation increased with repeat length, and that SPN loss occurred among SPNs with more than 150 repeats. The modeling found mutation rate and expansion-contraction-bias parameters that optimized the likelihood of the observed data from each person with HD, including the distribution of SPN CAG repeat lengths and SPN loss at the age of death and brain donation.

The distributions of observed CAG repeat lengths in caudate SPNs of donors with HD exhibited an unusual yet consistent shape, with a pronounced right tail consistently commencing at about 80 CAGs across many donors, such as shown in the plurality of plots 1612 of FIG. 16B. To better understand how these distributions might arise and evolve from the simple stepwise expansion-and-contraction process that biological studies have suggested is their primary mode of somatic mutation, stochastic models and simulations for the dynamics of the somatic expansion process were developed. An aim was to see whether such models could explain these observed distributions and the typical trajectories of SPN loss in HD particularly if, as the experiments suggested, the HTT CAG repeat becomes pathogenic when it is quite long, a length attained by few SPNs at any moment in time.

The following sections describe non-limiting examples of models that may be used by the expansion dynamics modeling module 154 of FIG. 1.

### Continuous-Time Markov Chains

Stochastic models were developed in which the mutation process in the HD-causing CAG repeat is a biased random walk. In each SPN, the probability of the CAG-repeat either expanding or contracting, within a given time interval t, is a function of its own current CAG-repeat length. Such memoryless models can be expressed as continuous-time Markov chains (CTMCs) in which the state space corresponds to the range of modeled repeat lengths and the transitions correspond to mutations that change the repeat length.

In these models, a state space ranging from CAG=0 to CAG=max_cag was used, where in practice max_cag was set to 500. In these models, the state corresponding to CAG=max_cag is treated as a “sink” state where once a cell reaches this repeat length, it will stay in this state indefinitely. In practice, this state is chosen to be beyond the range of repeat lengths where it is desired to model the dynamics of the somatic expansion process. This sink state is used to represent all cells that have reached very long CAG lengths. As discussed below, in most of these models, it is assumed that the majority of such cells have been lost.

Every CTMC process can be described by a rate matrix Q[i,j] that describes the probability of transitioning from state i to state j in unit time. In these models, the time unit is years. For each somatic expansion model, M, Q[i,j] is defined for Min terms of a smaller set of model parameters v for model M(v) and then fit these model parameters as described below. For any CTMC with rate matrix Q, there exists a unique matrix P(t) where the entries p[i,j]specify the probability that a cell with repeat length i at time zero has a repeat length j at time t. P(t) can be expressed as the matrix exponential

$$P ( t ) = e \hat{} tQ$$

which can be efficiently computed using the expm package in R. For a specific donor, if t is set equal to the age of brain donation and it is assumed that all cells started with a repeat length equal to the donor's inherited repeat length (inh_cag), then the function

$$f ( x ) = P ( t ) [ inh\_cag , x ]$$

gives the probability distribution of repeat lengths for this donor expected to be observed under the model M(v). Then, f(v), which is a function of the model parameters for M, is fit to the observed repeat length distribution for each donor to determine the best-fit estimate the model parameters for that donor.

### Generating a Rate Matrix Q for a Specific Model

As an illustrative example, consider a simple model with three parameters (p, r and T) where the rate of mutation is a linear function of the repeat length above a threshold T and each mutation results in either a one-repeat-unit CAG expansion with probability p or a one-repeat-unit CAG contraction with probability i-p. The mutation rate per unit time is modeled as:

$$μ ( x ) = r * max ( x - T , 0 )$$

where x is the current repeat length and T is the threshold below which no expansion or contraction occurs. The rate matrix Q[i,j] where 0<i,j<max_cag can be formed as:

$$\begin{matrix}Q [ i , i + 1 ] = p * μ ( i ) & for 0 <= max\_cag \\ Q [ i , i - 1 ] = ( 1 - p ) * μ ( i ) & for 0 < i <= max\_cag \\ Q [ i , i ] = - sum ( j ! = i , Q [ i , j ] ) & for 0 <= i <= max\_cag \\ Q [ i , j ] = 0 & otherwise\end{matrix} .$$

The rate matrix Q is a function of the model parameters (p, r and T). Each of these model parameters can either be fixed or be fitted from the data.

### Estimating the Amount of SPN Loss for Each Donor

Caudate SPNs are lost during the course of Huntington's disease (see FIG. 13). From the transcriptome data, the proportion of different cell types in the tissue samples from the anterior caudate in both HD donors and controls was estimated. It was found that in unaffected control donors, 46% (+/−0.06%) of the nuclei sampled were derived from SPNs, whereas the fraction in donors with HD ranged from less than 1% to 51%, with an observed dependence on disease progression as measured by CAP score or Vonsattel grade.

It was observed, based on the transcriptome data, that across persons with HD, the abundance of observed polydendrocytes (oligodendrocyte progenitor cells) tended to decline alongside that of SPNs, but that other cell types had stable proportions relative to one another, mainly expanding (as a fraction of the total) to the extent that SPNs and polydendrocytes were lost. As a result, both the SPNs and polydendrocytes were treated as cell types undergoing cell loss associated with disease progression.

To estimate the degree of SPN loss in each donor, the ratio of SPNs to cells of unaffected cell types (all cell types excluding SPNs and polydendrocytes) was compared. This ratio was further compared to the median ratio observed in 52 control donors. This yielded estimates of SPN loss ranging from 0% to almost 100%.

Uncertainty (noise) in these cell-loss estimates was introduced by sampling of the inherently non-uniform tissue in the caudate (e.g., sampling of different amounts of white matter vs. gray matter). For many donors there were two estimates of SPN fraction: one from the many-donor (“cell village”) experiments and another from deep resampling of individual donors alongside single-cell CAG-repeat-length measurement. The cell-loss estimates from the village experiments were used, whenever available, as these measurements were from a consistent anatomical site within the anterior caudate and they exhibited stronger relationships with each donor's CAP score and with neuropathologist determination of disease stage (Vonsattel grade).

### Incorporating Cell Loss Estimates in Repeat Expansion Models

As HD progresses, loss of SPNs is profound. Experimental measurements of CAG-repeat length distributions are measurements of the SPNs that are still present at the end of a brain donor's life, which will generally be a small fraction of the SPNs with which a donor began life. Any model should take into account this profound effect of attrition on the observed CAG-repeat length distributions.

A simple assumption may be made, rooted in the single-cell gene-expression data, that cell loss occurs specifically in the right tail of the CAG-length distribution, beyond the threshold (˜150 CAGs) at which gene-expression changes begin to become detectable. The details of this cell-death process and its precise probabilistic relationship to CAG-repeat length are not modeled, as the details of this relationship have little impact on the results. As described in more detail below, somatic expansion beyond 150 CAGs is extremely fast compared to somatic expansion at earlier stages, e.g., 40-50 CAGs.

### Fitting a Repeat Expansion Model for an Individual Donor

For each donor and each model M(v), it was desired to find the values of the model parameters v that best predicted the observed repeat length distribution and cell loss estimate for that donor. These model parameters defined the dynamics of the repeat mutation process in that donor.

Model fitting was implemented as an optimization problem using R. For each individual donor, the inherited repeat length (inh_cag), the donor's age at brain donation (d), and a vector of observed repeat lengths (x) from N SPN cells were used. Given a model M with a vector of fixed parameters and a vector of parameters to be fitted (theta), the optim package in R was used to find optimal values for theta. Both the Nelder-Mead algorithm and the L-BFGS-B methods implemented in the optim package in R were used, and both achieved comparable results. In the final reported analyses, the L-BFGS-B method in the optim package along with empirically derived parameter ranges were used to aid with rapid convergence of the model fitting.

The objective function optimized over was the log-likelihood of the observed repeat length distribution under the parameterized model, but with two modifications. First, all observed cells with a repeat length greater than max_cag were assigned to the last repeat-length bin (corresponding to max_cag). Second, the likelihood was adjusted to account for cell loss based on the donor-specific estimate of the fraction of dead (and thus unobserved) cells, estimated as described above. This cell-loss estimate was incorporated in the following way: from the number of observed cells and the loss estimate, the number of missing/unobserved cells was calculated, and these were added as pseudo-counts to the last bin (corresponding to max_cag). To prevent large estimates of cell loss from dominating the model fit, any estimate for cell loss greater than 90% was capped at 90%.

### Single-Jump Vs Multiple-Jump Models

Within the space of CTMC models, a variety of different models for somatic expansion were explored and evaluated. The analyses focused on a set of models with a specific form based on two quantities:
μ(x) a function giving the mutation rate as a function of the current repeat length; and
pₑₓₚ the probability of an expansion (vs a contraction), when a mutation occurs.

This approach formally models processes where the repeat length mutates by exactly one CAG unit at a time (either an expansion or contraction) with the probability of an expansion being pₑₓₚ and of a contraction being 1−pₑₓₚ. These are so-called “single-jump” models, in contrast to multiple-jump models that incorporate larger mutation events by assuming some probability distribution over the change in repeat length, conditional on the occurrence of a mutation.

Several multiple-jump models were evaluated, such as models where the change in the repeat length was drawn from a geometric distribution with a maximum jump size ranging from two to five CAG units. Compared to otherwise equivalent models with single-repeat jumps, these models generated distributions with higher variance. It was found that although the multiple-jump models fit the observed data well, the fits were not substantially better than the equivalent single-jump models. As a result, it was concluded that there was insufficient power to reject single-jump models in favor of multiple-jump models with the currently available data. Therefore, single-jump models were focused on instead.

It was reasoned that as long as most mutation events are short, single-jump models should have good predictive power, even if some mutations are longer than one unit. The results indicate that single-jump models are sufficient to explain the observed data, which is consistent with a model where most jumps are, in fact, short. There are also other lines of evidence suggesting that most repeat length mutations are likely to be short. In vitro studies of the mismatch repair mechanism suggest that mismatch repair is more efficient at recognizing small extrusions compared to larger ones, including in systems that are specifically based on the genes that are modifiers of HD onset. In addition, the smooth, unimodal shape of the distributions observed in the data, as well as other studies in humans and mice, are consistent with large jumps being rare, at least in the early phases of repeat expansion.

In the later, more rapid phases of somatic expansion, the increased overall rate could be driven either by a higher rate of mutation events, a larger average jump size per mutation, or some combination of these effects. The available data do not strongly distinguish among these possibilities, as their effects upon the repeat length distributions and dynamics are so similar.

### Sensitivity of Model Fitting to the Net Expansion Rate

Model fitting for both the single-jump and multiple-jump models was strongly driven by the goodness-of-fit to the net expansion rate, which is the product of the mutation rate and the mean jump length. For multiple-jump models, it was found that an increase in mutation rate could be offset by a reduction in the expected jump size, and vice versa, to create roughly equivalent model fits.

For single-jump models, the net expansion rate is:

$${exp}_{net} ( x ) = μ ( x ) * ( 2 * {p}_{exp} - 1 ) .$$

A formal assumption in these models is that although the expansion probability pₑₓₚ can vary between donors, it is fixed in each donor and is independent of the current repeat length.

Because the model fit is driven by the net expansion rate, it is difficult to distinguish between models where the mutation rate varies as a function of the repeat length, models where the expansion bias (pₑₓₚ) varies as a function of repeat length, or some combination. For simplicity, it was chosen to model pₑₓₚ as a constant in each donor and allow the mutation rate μ(x) to vary as a function of repeat length.

Whether the expansion bias (or jump size) changes with repeat length will likely need the benefit of a more complete mechanistic understanding of the somatic expansion process.

Single-Phase Vs. Two-Phase Models of Somatic Expansion

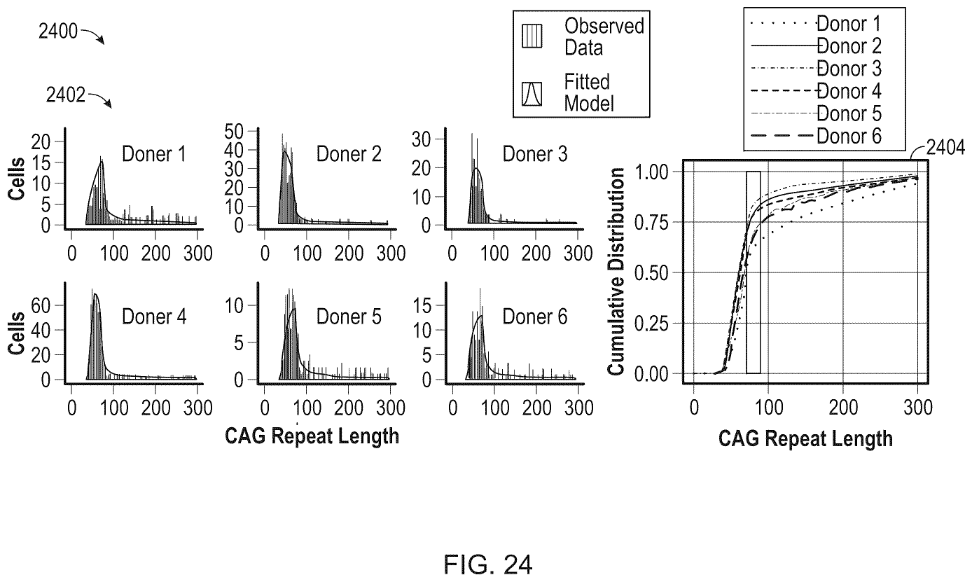

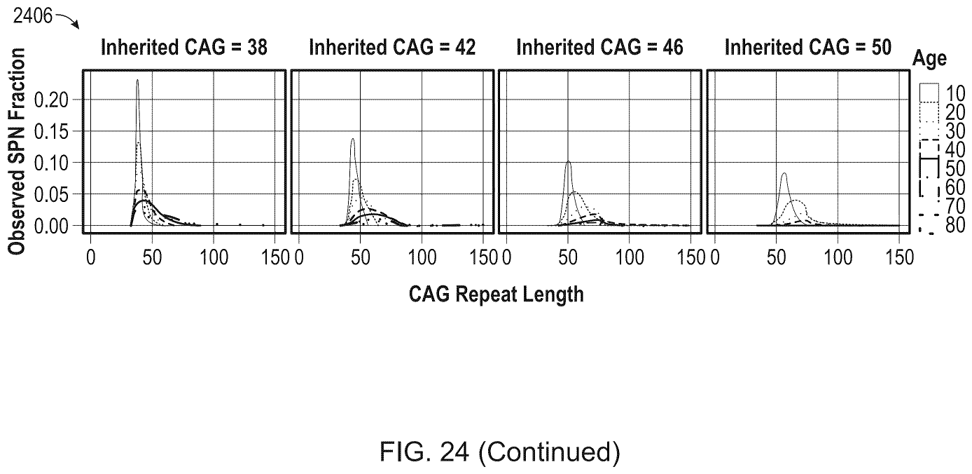

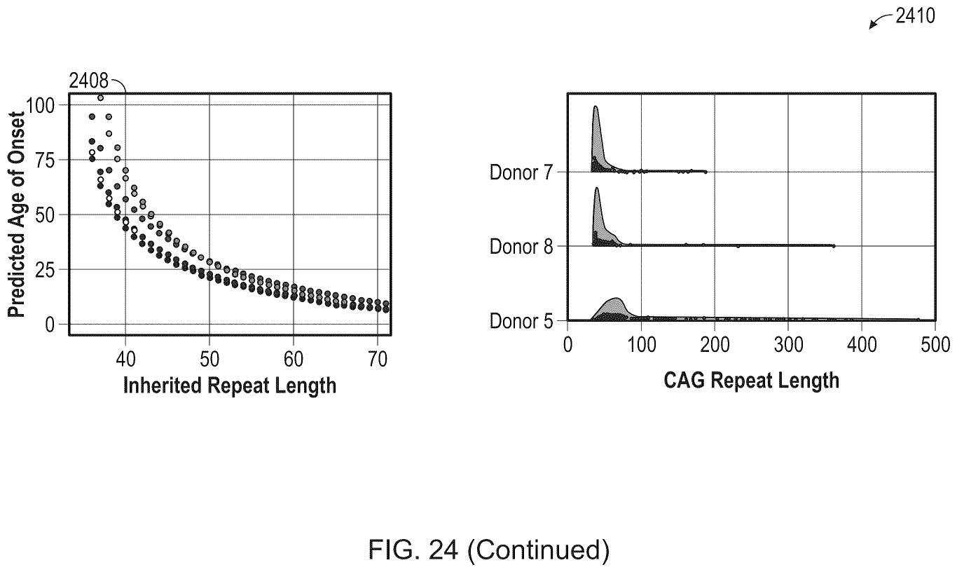

A number of different models for somatic expansion were implemented and investigated. Broadly, these models were divided into two categories: single-phase and two-phase. The investigation of the two-phase models was driven by the observation that the empirical cumulative distributions of the repeat lengths, across donors, tended to have a sharp bend (or “kink”) around 70-90 repeats (see the cumulative distribution plot 2404 of FIG. 24), suggestive of a rapid change in the dynamic expansion behavior in this range.

Though such a kink could be generated by a complex model assuming substantial SPN death at 70-90 CAGs (and further assuming that a subset of SPNs were immune to this for unknown reasons), the lack of apparent gene-expression changes across the 40-150 CAG range focused efforts on simpler models in which this kink was generated by repeat expansion dynamics. Moreover, even if many SPNs were lost at 70-90 CAGs, the surviving SPNs would also have repeats that continue to expand rapidly to reach the long repeat lengths (up to 800+ CAGs) observed in the data.

It has been well established that CAG repeats below approximately 35 CAGs exhibit very little instability and rarely lead to Huntington's disease. Indeed, no significant instability in the short alleles of vulnerable cell types in HD donors was observed. Accordingly, the models explored assumed that somatic expansion begins at a threshold (T1) of around 35 CAGs.

In practice, the models used set the mutation rate to zero below a fixed threshold. Since such models create a “sink” state at the instability threshold, it was found that it was practical to set the lower threshold, T1, to 33.5 to reduce the number of cells that would become trapped in this lower sink state.

Models that used a single threshold for somatic instability (T1), whether this threshold was fixed or fitted, were categorized as single-phase models. Models were also explored in which there was a second threshold (T2) at which the dynamic properties of the model were allowed to change, in particular to allow faster acceleration of the expansion rate beyond what could be achieved with common smooth functions and a single instability threshold. Models with this type of second threshold were categorized as two-phase models. Minimal smoothing was applied at the transition between the two phases, using instead the continuity of the mutation rate function (and in some models, its first derivative), as this was sufficient to explain the observed data. The underlying biological process quite likely does not exhibit as abrupt a transition as represented in these models.

### Models of Repeat Expansion

The main analyses presented here are based on two models, which are referred to as the TwoPhaseLinearModel and the TwoPhasePowerModel.

In the TwoPhaseLinearModel, the mutation rate varies as a piecewise linear function of the repeat length with three regimes. There is a threshold T1 below which the mutation rate is zero and a second threshold T2 separating the other two regimes. The mutation rate function for the TwoPhaseLinearModel is given by:

$$μ ( x ) = r 1 * max ( x - T 1 , 0 ) + r 2 * max ( x - T 2 , 0 )$$

where the parameters r1 and r2 are rate constants for the two non-zero regimes. The effective rate (slope) of the second of these regimes is r1+r2. In practice, T1 was fixed at 33.5, and T2 was fit from the data for each donor, as well as r1 and r2.

In the TwoPhasePowerModel, the mutation rate varies as a power function of the repeat length over three regimes, similar to the TwoPhaseLinearModel. The mutation rate function for the TwoPhasePowerModel is given by:

$$μ ( x ) = r 1 * {max ( x - T 1 , 0 )}^{a 1} + r 2 * {max ( x - T 2 , 0 )}^{a 2}$$

where r1 and r2 are rate constants similar to the previous model and a1 and a2 are (fitted) exponents for the two non-zero regimes. In practice, T1 was fixed at 33.5, and T2 was fit from the data for each donor (along with r1, r2, a1 and a2).

These two models were utilized in different ways. The TwoPhaseLinearModel was the simplest model that gave a good fit to the observed data. This model was used to estimate and compare parameter values between donors. The TwoPhasePowerModel was potentially over-parameterized but had the property of fitting the observed data well (better than the TwoPhaseLinearModel) at the cost of some over-fitting. It was found that using these over-fitted models allowed the computation of more reliable stochastic trajectories of the cells, which were useful for further analysis. Since there is a small degree of over-fitting in the TwoPhasePowerModel, comparison of the specific parameter values that generate the best fits for this model was avoided. Instead, the predicted trajectories were compared, as described further below.

As a point of comparison, a number of “one-phase” models were also evaluated. The one-phase models described here include the following, listed with their corresponding mutation rate functions:

$$\begin{matrix}OnePhaseLinearModel & μ ( x ) = r 1 * max ( x - T 1 , 0 ) \\ OnePhaseQuadraticModel & μ ( x ) = r 1 * max ( x - T 1 , 0 ) \hat{} 2 \\ OnePhasePowerModel & μ ( x ) = r 1 * max ( x - T 1 , 0 ) \hat{} a 1 \\ OnePhaseExponentialModel & μ ( x ) = r 1 * b 1 \hat{} k 1 * max ( x - T 1 , 0 )\end{matrix} .$$

In each of these models, T1 was fixed to 33.5, and the other parameters were fit from the data.

When a specific model is referred to by name herein, the repeat length threshold used for modeling cell loss is generally included after the name of the model, separated by slash. For example, TwoPhasePowerModel/150 would refer to the two-phase power model fitted using a cell loss threshold of 150 CAGs. The cell loss threshold is the minimum repeat length at which the cell loss is assumed to begin to occur, as described previously.

### Comparison of Different Models of Somatic Expansion

Across all the donors whose SPNs were deeply sampled with CAG-repeat length measurements, the two-phase model provided better fits than the one-phase models.

Two qualities of the models that were able to best fit the observed data were observed: first, that the models allowed the rate of somatic expansion (in CAGs/year) to increase as a super-linear function of the current repeat length; and second, that the best models were two-phase models that allowed the dynamics of the expansion process to further accelerate around 70-90 CAGs. It was found that the exact functional forms used in the two-phase models were not as impactful (given the available data) as allowing the dynamics to accelerate at this repeat length threshold. For simplicity, two-phased models were utilized that used the same functional form for both phases.

It was found useful to evaluate these models based on their predicted net mutation rate curves (in CAGs/year) as a function of repeat length. It was observed that in this framework, both the one-phase and two-phase models appeared to converge to a common net mutation rate curve, best represented by the two-phase power models, which adhere most closely to the shape of the observed data. As a consequence, most of the analyses were based on two models: the two-phase power model, which had the best overall fit of the models tried, and the two-phase linear model. Both of these models generated good fits across donors. The two-phase power model, which provided the best fits overall, was used when comparing the expansion trajectories across donors. The two-phase linear model was used in contexts where it was desired to directly compare the fitted parameter values between donors to avoid potential over-fitting of the two-phase power model.

The use of two-phase models was not intended to suggest that there is some additional, previously unknown biological process contributing to somatic expansion. The introduction of two phases appeared to better model the apparent shift in the observed net rate of expansion observed in the data. In principle, many different phenomena could account for this increase in expansion rate, including interactions with local chromatin structure or changes in the jump size per mutation event as the repeat length increases. Understanding the mechanism that underlies this aspect of the somatic expansion dynamics will be an area of future research.

### Characterizing the Changes in Somatic Expansion Between Phase a and Phase B

In the model for HD neuropathology at the single-cell level, the initial stages prior to the emergence of gene dysregulation are partitioned into two phases: phase A, which corresponds to the slow expansion phase predicted by the two-phase models of somatic expansion, and phase B, which corresponds to the faster expansion phase prior to the beginning of transcriptomic dysregulation around 150 CAGs.

To quantify the transition between and the properties of these two phases, the two-phase linear model was relied on. While the two-phase power model provides a better fit overall and appears to better capture the overall trajectory, the two-phase linear model produces fits to the data that are similar and has some advantages for parameter estimation. First, the parameters are easier to compare between the phases using the two-phase linear model, as each phase is a simple linear function representing a kind of average behavior across the phase. Second, because the two-phase linear model has fewer parameters, it is easier to interpret and less vulnerable to over-fitting.

The parameters of the two-phase linear model were fitted to each donor. The parameters for the rate of expansion (pₑₓₚ=0.676+/−0.011) and for the threshold to transition between phase A and phase B (threshold2=72.2+/−2.53) were quite stable across donors. In different models, the value for threshold2 across donors tended to be similar, but the actual value inferred by different models varied depending on the functional form used for μ(x) in each model and whether a given model allows a greater or lesser degree of smoothing around the transition. As a result, although the models use a discrete value to fit the transition, it is recommended that the transition between phase A and phase B should be interpreted as a range. A consensus threshold of 80 CAGs was used to represent the midpoint of this transition range in downstream analyses.

The effective rate of expansion in each phase is rate1*(2*pₑₓₚ−1) for phase A and (rate1+rate2)*(2*pₑₓₚ−1) for phase B. The phase A expansion rate in the six deeply sequenced donors was 3.51% (+/−0.83%) CAGs/year, and the phase B expansion rate was 57.6% (+/−12.0%). The ratio of these two mean expansion rates (phase B/phase A=16.4) was used as the consensus estimate of the change in expansion rates above and below the transition between phases A and B.

Results have Limited Sensitivity to the CAG Repeat Threshold for SPN Death

As described above, these donor-specific estimates of SPN cell loss were incorporated in these models of somatic expansion dynamics by assuming a minimum repeat length threshold for cell loss. Assumptions regarding relationships between repeat length and cell loss above this threshold were avoided. Instead, it was assumed that cell loss would be at (or near) zero below this threshold.

To evaluate the sensitivity of these models to mis-estimation of this cell loss threshold, the models were run assuming a cell loss repeat-length threshold of 150 CAGs and a higher repeat-length threshold of 300 CAGs. The models were relatively insensitive to the exact repeat-length threshold for cell loss. Since the models predict that the rate of somatic expansion is fast when the repeat length exceeds 100 CAGs, changing the assumed minimum repeat-length threshold for cell loss had a minimal effect on the overall trajectory and rate of cell loss.

### Predicting the Trajectory of Somatic Expansion Across Time

The somatic expansion models fit to the data from each donor allowed the distribution of repeat lengths in each donor to be projected both backwards in time and forwards in time from the date of brain donation.

### Cell Loss Trajectories for Caudate SPNs in Individual Donors

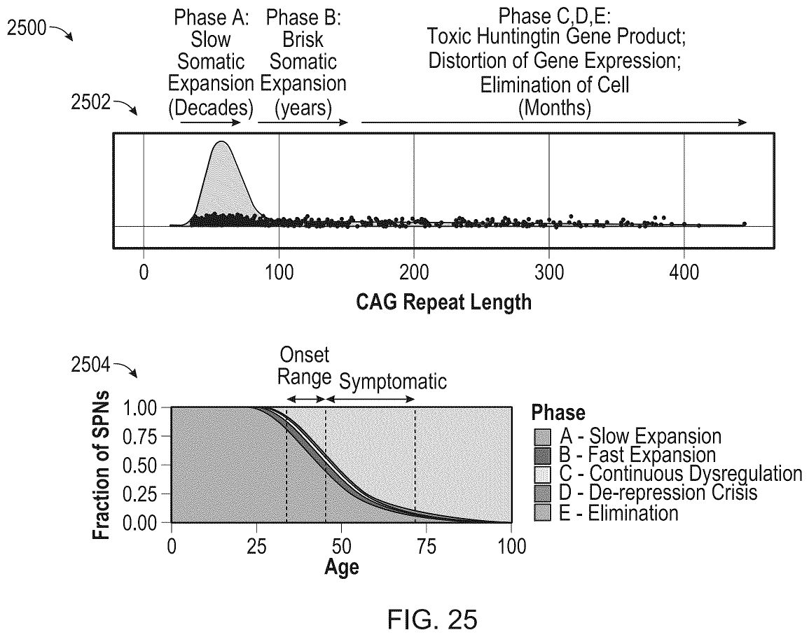

The model for neuronal pathology consists of five phases, as elaborated below with respect to FIG. 25. Although the repeat-length thresholds at which an individual SPN transitions between these phases are not precise (for example, the transition from slow expansion to fast expansion happens within a range from about 70-90 CAGs), the fraction of SPNs in each of these phases of pathology over time were estimated based on the following repeat-length thresholds: a transition from phase A to B (80 CAGs), a transition from phase B to C (150 CAGs), a transition from phase C to D (250 CAGs), and a transition to phase E (500 CAGs). Because the rate of expansion is rapid when the repeat is highly expanded (greater than 100 CAGs), these visualizations are not sensitive to the precise thresholds used; different thresholds would produce qualitatively similar trajectories.

### Estimating Somatic Expansion Prior to Future Toxicity at Age of Onset

A question of great interest for patients and for the design of clinical trials is to estimate, at any point in disease, how many of a patient's SPNs have not yet reached the toxic phase and thus will undergo further somatic expansion to reach this phase. To estimate this from the somatic expansion models, the following methodology was used.

Since actual age-at-onset data was not available for many of the donors investigated, an approximate age-at-onset was first estimated as the predicted age at which 30% of the donor's SPNs would have reached a repeat length of at least 250 CAGs (using somatic expansion model TwoPhasePowerModel/150). For this age, the fraction of the donor's SPNs predicted to have a repeat length below 150 CAGs (not yet exhibiting toxicity) in comparison to the fraction predicted to have a repeat length under 500 CAGs (a conservative threshold for SPNs that would be alive/observable) was estimated. Across the six donors with sufficient data for modeling, the mean of this quantity was 91.5% (+/−3.8%).

This estimate was largely insensitive to the threshold used for estimated age-at-onset, with nearly identical results when using 20% or 40% of SPNs with repeats longer than 150 CAGs. The estimate was also largely insensitive to the age at which the estimate is made. This quantity (fraction of a donor's SPNs predicted to have repeat length below 150 CAGs compared to the fraction predicted to have repeat length under 500 CAGs) was estimated across all ages (up to 100 years old), covering effectively all disease stages, and the minimum for each donor was computed. The mean across donors was 86.5% (+/−6.2%).

### Estimating Mean Somatic Expansion Rates

To estimate the mean number of years for a typical cell to expand from 40 to 60 to 80 CAGs, the TwoPhasePowerModel/150 was used with the following analysis. First, adjusted models were computed for each donor as if they had inherited a repeat of length 40. Then, the age at which the median CAG in each donor would reach 60 or 80 CAGs was estimated. The mean age of reaching 60 CAGs was 50.7 (+/−13.5) years. The additional time to reach 80 CAGs was 11.7 (+/−1.5) years. Reaching 150 CAGs was an additional 3.4 (+/−0.5) years. These results highlight that in these models, donors vary greatly in their rate of progression, but there is less variability in the projected trajectory of progression. Inter-donor variability is larger under these models.

For comparison, the same analysis was performed, but with modeling each donor as starting with an inherited repeat length of 42 CAGs. In this case, the mean age of reaching 60 CAGs was 37.8.

FIG. 24 shows an example 2400 of modeling data for repeat expansion dynamics. The example 2400 includes a first set of plots 2402 depicting distributions of CAG repeat length measurements in SPNs from six representative donors overlaid with stochastic models for which parameters such as mutation rate have been fitted to each donor's repeat-length and SPN-loss data.

The example 2400 further includes a cumulative distribution plot 2404 of the experimentally measured CAG repeat lengths for SPNs from these same donors. The shaded region highlights the range (70-90 CAGs) over which somatic expansion appears to greatly accelerate. The example 2400 further includes a second set of plots 2406 showing the effect of changing a single variable (germline CAG repeat length) in the model for a typical donor (with a true inherited CAG of 43), keeping the other fitted parameters fixed. Each curve indicates the predicted CAG repeat length distribution for surviving SPNs at each decade (ages 10 to 80).

The example 2400 further includes a modeling plot 2408 that estimates the relationship between inherited germline CAG repeat length and age at clinical motor onset. As a proxy for age of onset, the predicted time at which 25% of a donor's SPNs have reached a repeat length of 300 or more CAGs was used. Each donor's age of onset proxy was estimated at different hypothetical inherited repeat lengths. The shapes of the resulting curves in the modeling plot 2408 closely approximate the known relationship between inherited repeat length and age of HD onset.

The example 2400 further includes a third set of plots 2410 depicting observed CAG repeat length distributions for two donors who passed away prior to clinical motor onset of HD (Donor 7 and Donor 8) and a symptomatic donor (Donor 5). Donor 7 was recorded as “at risk,” and Donor 8 had not manifested motor symptoms based on a review of their medical records. The data for Donor 7 and Donor 8 depicted in the third set of plots 2410 reveals that their SPNs had undergone substantial somatic expansion prior to clinical motor onset, but few of each patient's cells had expanded beyond 150 CAGs.

The most challenging aspect to explain of the repeat length data to explain is its armadillo shape—the simultaneous presence of a large majority of SPNs with 40-100 CAGs, and a small minority of SPNs with far more (100-800+) CAGs. All the donors analyzed exhibited this transition across the same CAG-repeat length range of about 70-90 CAGs, as shown in the cumulative distribution plot 2404. Models in which the increase in the mutation rate was a linear, quadratic, higher-order polynomial, or log-normal function of repeat length did not generate this shape. However, models with two phases of expansion—a slow phase (phase A) that transitioned into a much faster phase (phase B)—generated data that closely matched the experimental data (e.g., the first set of plots 2402). The models estimated this transition as occurring over a similar repeat length interval (70-90 CAGs) in each donor, with the mutation rate increasing at least ten-fold over this range. At this nucleotide length scale (200+bp), otherwise mobile slip-out structures (see FIG. 23) may be separated, with increasing likelihood, by an intervening nucleosome, greatly increasing the likelihood that they are surveilled by MMR complexes before they resolve on their own.

Explaining the experimental data using these models did not utilize assumptions of single-cell heterogeneity in mutation rates. It was found that asynchronous SPN toxicity could be explained by the asynchronous passage of SPNs from phase A to the subsequent, faster phase B. This asynchronicity arose simply from the observations that length change mutations were initially rare events (occurring less than once per year per cell across 36-55 CAGs), and upon occurring, such mutations increased the probability of subsequent mutations.

A fundamental relationship in HD is the association between longer inherited alleles and earlier HD onset, which is steep for inherited alleles of 36-50 repeats and has long been thought to reflect increasing mHTT toxicity in this range. The simulations also produced this relationship, but for a different reason: slightly longer inherited alleles bypassed the CAG-repeat lengths at which somatic expansion is most slow, as indicated in the second set of plots 2406. Moreover, simulations suggested that the earlier loss of iSPNs relative to dSPNs could be explained by a modestly higher (˜15%) rate of somatic expansion.

A long-standing mystery about HD involves the long latent period (generally decades) in which persons have no apparent symptoms (ISS Stage 0). The simulations predicted that persons in this stage might in fact have substantial somatic expansion, but with only a small fraction of their SPNs having completed the slow expansion phase (phase A) and entered subsequent phases. To evaluate this, caudate tissue from two persons with HD who had passed away and contributed their brains for research prior to motor symptom onset and/or without apparent neuropathology upon autopsy were examined (e.g., Donor 7 and Donor 8 described above). Distributions of CAG-repeat lengths in their SPNs indeed exhibited substantial somatic expansion but included very few cells with long (greater than 100) expansions, as shown in the third set of plots 2410.

It was also found that explaining a long-puzzling feature of HD the rapidly escalating rate of caudate atrophy after symptom onset-did not utilize common assumptions of a non-cell-autonomous disease escalating process (such as inflammation or spreading prions). Rather, the escalating period corresponded to the period in which the bulk of a person's SPNs reached the end of phase A and more quickly traversed the subsequent, pathological phases.

The simulations suggest that the average SPN in a person with the most common HD-causing inherited allele (42 repeats) spends approximately 96% of its life below the threshold of 150 CAG repeats at which distorted gene expression appears to commence, e.g., with what the experiments suggest is an innocuous mutant huntingtin gene.

### A Model for HD Pathogenesis

The results described above indicate that an SPN's own CAG repeat is sufficient for its pathology and is consequential only upon becoming quite long (greater than 150 CAGs). A somatic long expansion, asynchronous toxicity (SLEAT) model is therefore proposed for the pathology of SPNs in HD. The SLEAT model includes a series of phases, each driven cell-autonomously by each neuron's own expanding HTT allele.

FIG. 25 shows an overview 2500 of a model for neuropathology in HD. The overview 2500 includes a graphical representation 2502 of the CAG repeat length (horizontal axis) with respect to annotated phases. Individual neurons pass asynchronously through five pathological phases, spending more than 95% of their lives in a long period of DNA repeat expansion (a “ticking DNA clock,” phases A and B) with a biologically harmless (but unstable) HTT gene. Individual neurons asynchronously exit phase A and proceed through the subsequent, faster phases (phases C, D, and E).

The overview 2500 further includes a modeled prediction 2504 of the fraction of SPNs in each of the five phases of HD. The estimated trajectories are based on the data from a representative donor. The indicated ranges for clinical motor onset and escalating symptoms are approximate. The illustrated onset range, representing between 20% to 50% SPN loss, is inferred from available medical records of the patients analyzed.

The proposed phases in HD pathology at the single-neuron level are also summarized in Table 11 below. Time estimates are for persons who inherit the more common HD-causing alleles (40 to 45 CAG repeats).

TABLE 11

| | | | | Gene expression changes associated | | |
|---|---|---|---|---|---|---|
| | | | | with neuron's own HTT CAG repeat | | |
| | | | | length (relative to nearby neurons in | | |
| | CAG | Somatic | Time in | the same tissue) | | |
| | repeat | expansion | phase | Genes | | |
| Phase | length | dynamic | (est.) | affected | Dynamic | Functional theme |
| A slow repeat | 36 to 80 | Slow (initially <1/ | Decades | — | — | — |
| expansion | | yr) but | | | | |
| | | increasing | | | | |
| B brisk repeat | 80 to 150 | Fast | Years | — | — | — |
| expansion | | | | | | |
| C continuous | >150 | Fast | Months | Hundreds | Gradual, | Erosion of cell |
| change | | | | | escalating | identity |
| D de-repression | >>150 | Fast | Weeks | Hundreds | Discrete, | De-repression of |
| | | | | more | sudden | genes normally |
| | | | | | | expressed by other |
| | | | | | | cell types |
| E eliminated | | | | | | |

In the first phase (phase A, when a neuron has 36 to 80 CAGs), an SPN undergoes decades of slow-but-accelerating repeat expansion. For example, it may take a first number of years to expand from 40 to 60 CAGs, and then a second number of years to expand from 60 to 80. Phase A could be compared to a slowly and capriciously ticking DNA clock.

As a neuron enters the second phase (phase B, 80 to 150 CAGs), the rate of expansion greatly accelerates, and the tract may now expand to 150 CAGs in just a few years. Still, as in phase A, the neuron's HTT CAG repeat does not appear to affect its own gene expression. Phase B could be compared to a more rapidly, predictably ticking DNA clock.

As a neuron enters the third phase (phase C, 150+ repeat units), hundreds of genes begin to change in expression levels. These changes are initially tiny, but they escalate alongside further repeat expansion (see FIGS. 17-20), eroding gene-expression features of SPN identity (e.g., as shown in the plot 2004 of FIG. 20).

In its fourth phase (phase D), an SPN de-represses scores of genes that are typically expressed in other neural cell types or in embryonic development. Phase-D neurons also express CDKNV2A and CDKNV2B, which encode proteins that promote senescence and apoptosis.

In the final phase, an SPN is eliminated (phase E). Such cells do not appear in CAG length and gene expression data, although their earlier loss is apparent in the declining numbers of SPNs (see FIG. 13) and in gene expression changes in remaining cells of all types (including SPNs), which correlated with earlier SPN loss.

Individual SPNs appear to enter the fast phases (B, C, D, and E) at different times, an asynchrony which the modeling suggests can be explained largely by the variable amounts of time that individual neurons take to traverse phase A. Phase A introduces this asynchrony because each neuron's expansion results from low-frequency stochastic length change mutations (initially occurring less than once per year), with each expansion event increasing the likelihood of subsequent such events.

### Example 3: Methods of Treating Huntington's Disease

In example implementations, via the techniques described herein, it has been established for the first time that Huntington's Disease (HD) is not caused by expression of HTT, but a sequence of events by which inherited repeat-expanded alleles in the Huntingtin gene lead to loss of striatal projection neurons (SPNs). First, alleles that are 35 CAGs or longer (the threshold for being disease-causing) expand selectively in SPNs, but not other caudate cell types. The rate of expansion is initially slow but increases as repeat expansions grow. Repeats may expand for many decades with little if any cell-autonomous biological consequence (though neurons and other cells may be beginning to experience secondary effects from the loss of neurons that have expanded precociously). Finally, as the repeat length passes 180, neuron somatic repeat expansion occurs selectively in post-mitotic neurons, suggesting that the process is not related to DNA replication, and instead arises from cells' efforts to repair mutations that arise during a neuron's long post-mitotic life. In example implementations, treatment comprises administering one or more agents that inhibit somatic expansion in SPNs. In example implementations, the one or more agents restore or enhance DNA repair in SPNs.

The focus of almost all therapies in advanced clinical development for HD is on lowering HTT expression; approaches including antisense oligonucleotides, small interfering RNAs, splicing modulation, or gene editing have been used. Under conventional models for HD pathology, HTT lowering has a compelling rationale: if inherited HD-causing alleles encode a toxic protein (or become toxic after just modest somatic expansion), and if the cell-biological process by which such alleles lead to neuronal death is decades-long, then even a partial reduction in HTT production might greatly postpone HD onset or progression. However, HTT-lowering treatments have so far been unsuccessful in HD clinical trials.

The SLEAT model (see FIG. 25) suggests a challenge for HTT-lowering as an approach: at any time, very few SPNs may actually have a toxic HTT protein from whose lowering they could benefit. Moreover, at the same time, most neurons may be deriving positive biological function from HTT. Even once an SPN arrives at cell-biological toxicity (phases C and D described above) and may benefit from HTT lowering, its expected lifetime (if untreated) may be months rather than decades. In short, HTT-directed therapeutic efforts should address the possibility that HTT toxicity is brief, asynchronous, and intense, rather than long, synchronous and indolent.

The conclusion that HD pathogenesis is a DNA process for >95% of a neuron's life might encourage greater focus on slowing somatic expansion. Experimental reduction in the function of MMR genes (includingMSH3, MSH2, MSH6 and PMS1) can stabilize DNA repeats in mice and/or cultured cells and thus might pre-empt the somatic-genetic cause of HD pathology. However, much uncertainty has surrounded the therapeutic window that such an approach could have. The present results suggest that the therapeutic window for a therapy that slows somatic expansion might widen: if a cell spends 95% of its life in phase A, then even modestly slowing somatic expansion might greatly postpone HD symptom onset.

The results described herein predict that even upon symptom onset, most (more than 90% of) future neuronal toxicity will occur after future somatic expansion (see FIGS. 24 and 25). A future somatic expansion-directed therapy thus might be able to slow or stop HD progression even in persons who already have early HD symptoms. This would allow the efficacy of such therapy to be evaluated in patients with HD symptoms, which is a faster and more straightforward path to clinical evaluation than a long-term prevention trial.

The expansion dynamic described herein might also apply in other repeat expansion disorders. More than 40 human diseases are caused by inherited expansions of DNA repeats in protein-coding sequences, introns, UTRs, or promoters. Several of these diseases involve age-associated mosaicism and mid-life onset. Many, including Myotonic dystrophy 1, X-linked dystonia Parkinsonism, Friedrich ataxia, and six forms of spino-cerebellar ataxia (SCA1, SCA2, SCA3, SCA6, SCA7, SCA11), are also (like HD) delayed or hastened by common genetic variation at genes that regulate somatic instability. If these disorders share a dynamic in which pathological changes are initiated by long somatic repeat expansions, then a therapy that slows somatic expansion might prevent many human repeat expansion disorders.

In example implementations, unique gene expression in SPNs that have CAG somatic expansion greater than 180 CAGs proceeds death of the neurons. In example implementations, treating HD comprises administering one or more agents that modulate one or more genes differentially expressed in SPNs that have CAG somatic expansion greater than 180 CAGs. Example agents that modulate these genes can be any drug already approved for the treatment of a disease that is not an expansion gene (e.g., FDA approved drugs), including the example agents described below.

### Antibodies

In example implementations, one or more overexpressed genes in striatal projection neurons (SPNs) that have CAG somatic expansion greater than 180 CAGs are targeted using antibodies. In example implementations, surface proteins are targeted. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the disclosure, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgAQ1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by R pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds include, without limitation, affibodies based on the Z-domain of staphylococcal protein A, a three-helix bundle of 58 residues providing an interface on two of its alpha-helices; engineered Kunitz domains based on a small (ca. 58 residues) and robust, disulfide-crosslinked serine protease inhibitor, typically of human origin (e.g. LACI-D1), which can be engineered for different protease specificities; monobodies or adnectins based on the 10th extracellular domain of human fibronectin III (10 Fn3), which adopts an Ig-like beta-sandwich fold (94 residues) with 2-3 exposed loops, but lacks the central disulfide bridge; anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms; DARPins, designed ankyrin repeat domains (166 residues), which provide a rigid interface arising from typically three repeated beta-turns; avimers (multimerized LDLR-A module); and cysteine-rich knottin peptides.

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present disclosure and antibodies of the disclosure bind with a range of affinities, for example, 100 nM or less, 75 nM or less, 50 nM or less, 25 nM or less, for example 10 nM or less, 5 nM or less, 1 nM or less, or in implementations, 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}i domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment, which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulfide bridge at the hinge region; (ix) single chain antibody molecules (e.g., single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (V_{H}-Cₕ1-V_{H}-Cₕ1) which, together with complementary light chain polypeptides, form a pair of antigen binding regions.

As used herein, a “blocking” antibody or an antibody “antagonist” is one which inhibits or reduces biological activity of the antigen(s) it binds. In some implementations, the blocking antibodies or antagonist antibodies or portions thereof described herein completely inhibit the biological activity of the antigen(s).

Antibodies may act as agonists or antagonists of the recognized polypeptides. For example, the present disclosure includes antibodies which disrupt receptor/ligand interactions either partially or fully. The disclosure features both receptor-specific antibodies and ligand-specific antibodies. The disclosure also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific implementations, antibodies are provided that inhibit ligand activity or receptor activity by at least 95%, at least 90%, at least 85%, at least 80%, at least 75%, at least 70%, at least 60%, or at least 50% of the activity in absence of the antibody.

In some implementations, receptors are targeted with antibodies that block ligand binding. The disclosure also features receptor-specific antibodies which both prevent ligand binding and receptor activation as well as antibodies that recognize the receptor-ligand complex. Likewise, encompassed by the disclosure are neutralizing antibodies which bind the ligand and prevent binding of the ligand to the receptor, as well as antibodies which bind the ligand, thereby preventing receptor activation, but do not prevent the ligand from binding the receptor. Further included in the disclosure are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art.

The antibodies as defined for the present disclosure include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present disclosure are transmembrane proteins, assays that use the soluble forms of these proteins rather than full-length protein can be used, in some implementations. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In example implementations, one or more overexpressed genes in striatal projection neurons (SPNs) that have CAG somatic expansion greater than 180 CAGs are targeted using aptamers designed to bind to one of the ligand-receptor proteins. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In some implementations, RNA aptamers may be expressed from a DNA construct. In other implementations, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 year) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are may include oligonucleotides containing nucleotide derivatives chemically modified at the 2′ position of ribose, 5 position of pyrimidines, and 8 position of purines; various 2′-modified pyrimidines; or highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further implementations, the oligonucleotides comprise modified sugar groups, for example, one or more of the hydroxyl groups is replaced with halogen, aliphatic groups, or functionalized as ethers or amines. In one implementation, the 2′-position of the furanose residue is substituted by any of an O-methyl, O-alkyl, O-allyl, S-alkyl, S-allyl, or halo group. Other modifications are known to one of ordinary skill in the art. In some implementations, aptamers include aptamers with improved off-rates. In some implementations aptamers are chosen from a library of aptamers. Aptamers are also commercially available. In some implementations, the present disclosure may utilize any aptamer containing any modification as described herein.

### Genetic Modifying Agents

In some implementations, one or more overexpressed genes in striatal projection neurons (SPNs) that have CAG somatic expansion greater than 180 CAGs are targeted with a genetic modifying agent configured to modify the one or more of the target genes. The genetic modifying agent may comprise a programmable nuclease, such as, a CRISPR system, a zinc finger nuclease system, a TALEN, or a meganuclease. In some implementations, a polynucleotide of the present disclosure described elsewhere herein can be modified using a genetic modifying agent. In example implementations, the genetic modifying agent is administered using a vector, such as a viral vector or liposome. In example implementations, the genetic modifying agent is targeted to neurons. In example implementations, the genetic modifying agent is administered directly to the brain.

### CRISPR-Cas

In one example implementation, the genetic modifying agent is a CRISPR-Cas system. CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence in one or more of the target genes. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to a desired target site at or near the one or more target genes. Additionally, CRISPR systems can be used in vivo.

In general, a CRISPR-Cas or CRISPR system as used in herein refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some implementations, the CRISPR-Cas system that can be used to modify a polynucleotide of the present disclosure described herein can be a Class 1 CRISPR-Cas system. In some implementations, the CRISPR-Cas system that can be used to modify a polynucleotide of the present disclosure described herein can be a Class 2 CRISPR-Cas system.

### Class 1 CRISPR-Cas Systems

In some implementations, the CRISPR-Cas system that can be used to modify a polynucleotide of the present disclosure described herein can be a Class 1 CRISPR-Cas system. Class 1 CRISPR-Cas systems are divided into types I, II, and IV. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Type IV CRISPR-Cas systems are divided into 3 subtypes (IV-A, IV-B, and IV-C). Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems.

The Class 1 systems typically comprise a multi-protein effector complex, which can, in some implementations, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some implementations, multiple copies of RAMPs can be present. In some implementations, the Class I CRISPR-Cas system can include 1, 2, 3, 4, 5, 6, 7, 8, 9, 10, 11, 12 or more Cas5, Cas6, and/or Cas 7 proteins. In some implementations, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some implementations, also include a large subunit. The large subunit can be composed of or include a Cas8 and/or Cas10 protein. Class 1 CRISPR-Cas system effector complexes can, in some implementations, include a small subunit (for example, Cas11).

In some implementations, the Class 1 CRISPR-Cas system can be a Type I CRISPR-Cas system. In some implementations, the Type I CRISPR-Cas system can be a subtype I-A CRISPR-Cas system. In some implementations, the Type I CRISPR-Cas system can be a subtype I-B CRISPR-Cas system. In some implementations, the Type I CRISPR-Cas system can be a subtype I-C CRISPR-Cas system. In some implementations, the Type I CRISPR-Cas system can be a subtype I-D CRISPR-Cas system. In some implementations, the Type I CRISPR-Cas system can be a subtype I-E CRISPR-Cas system. In some implementations, the Type I CRISPR-Cas system can be a subtype I-F1 CRISPR-Cas system. In some implementations, the Type I CRISPR-Cas system can be a subtype I-F2 CRISPR-Cas system. In some implementations, the Type I CRISPR-Cas system can be a subtype I-F3 CRISPR-Cas system. In some implementations, the Type I CRISPR-Cas system can be a subtype I-G CRISPR-Cas system. In some implementations, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some implementations, the Class 1 CRISPR-Cas system can be a Type III CRISPR-Cas system. In some implementations, the Type III CRISPR-Cas system can be a subtype III-A CRISPR-Cas system. In some implementations, the Type III CRISPR-Cas system can be a subtype III-B CRISPR-Cas system. In some implementations, the Type III CRISPR-Cas system can be a subtype III-C CRISPR-Cas system. In some implementations, the Type III CRISPR-Cas system can be a subtype III-D CRISPR-Cas system. In some implementations, the Type III CRISPR-Cas system can be a subtype III-E CRISPR-Cas system. In some implementations, the Type III CRISPR-Cas system can be a subtype III-F CRISPR-Cas system.

In some implementations, the Class 1 CRISPR-Cas system can be a Type IV CRISPR-Cas-system. In some implementations, the Type IV CRISPR-Cas system can be a subtype IV-A CRISPR-Cas system. In some implementations, the Type IV CRISPR-Cas system can be a subtype IV-B CRISPR-Cas system. In some implementations, the Type IV CRISPR-Cas system can be a subtype IV-C CRISPR-Cas system.

The effector complex of a Class 1 CRISPR-Cas system can, in some implementations, include a Cas3 protein that is optionally fused to a Cas2 protein, a Cas4, a Cas5, a Cas6, a Cas7, a Cas8, a Cas10, a Cas11, or a combination thereof. In some implementations, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some implementations, the CRISPR-Cas system is a Class 2 CRISPR-Cas system. Class 2 systems are distinguished from Class 1 systems in that they have a single, large, multi-domain effector protein. In certain example implementations, the Class 2 system can be a Type II, Type V, or Type VI system. Each type of Class 2 system is further divided into subtypes. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the RuvC-like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some implementations, the Class 2 system is a Type II system. In some implementations, the Type II CRISPR-Cas system is a II-A CRISPR-Cas system. In some implementations, the Type II CRISPR-Cas system is a II-B CRISPR-Cas system. In some implementations, the Type II CRISPR-Cas system is a II-C1 CRISPR-Cas system. In some implementations, the Type II CRISPR-Cas system is a II-C2 CRISPR-Cas system. In some implementations, the Type II system is a Cas9 system. In some implementations, the Type II system includes a Cas9.

In some implementations, the Class 2 system is a Type V system. In some implementations, the Type V CRISPR-Cas system is a V-A CRISPR-Cas system. In some implementations, the Type V CRISPR-Cas system is a V-B1 CRISPR-Cas system. In some implementations, the Type V CRISPR-Cas system is a V-B2 CRISPR-Cas system. In some implementations, the Type V CRISPR-Cas system is a V-C CRISPR-Cas system. In some implementations, the Type V CRISPR-Cas system is a V-D CRISPR-Cas system. In some implementations, the Type V CRISPR-Cas system is a V-E CRISPR-Cas system. In some implementations, the Type V CRISPR-Cas system is a V-F1 CRISPR-Cas system. In some implementations, the Type V CRISPR-Cas system is a V-F1 (V-U3) CRISPR-Cas system. In some implementations, the Type V CRISPR-Cas system is a V-F2 CRISPR-Cas system. In some implementations, the Type V CRISPR-Cas system is a V-F3 CRISPR-Cas system. In some implementations, the Type V CRISPR-Cas system is a V-G CRISPR-Cas system. In some implementations, the Type V CRISPR-Cas system is a V-H CRISPR-Cas system. In some implementations, the Type V CRISPR-Cas system is a V-I CRISPR-Cas system. In some implementations, the Type V CRISPR-Cas system is a V-K (V-U5) CRISPR-Cas system. In some implementations, the Type V CRISPR-Cas system is a V-U1 CRISPR-Cas system. In some implementations, the Type V CRISPR-Cas system is a V-U2 CRISPR-Cas system. In some implementations, the Type V CRISPR-Cas system is a V-U4 CRISPR-Cas system. In some implementations, the Type V CRISPR-Cas system includes a Cas12a (Cpf1), Cas12b (C2c1), Cas12c (C2c3), Cas12d (CasY), Cas12e (CasX), Cas14, and/or Cas(D.

In some implementations the Class 2 system is a Type VI system. In some implementations, the Type VI CRISPR-Cas system is a VI-A CRISPR-Cas system. In some implementations, the Type VI CRISPR-Cas system is a VI-B1 CRISPR-Cas system. In some implementations, the Type VI CRISPR-Cas system is a VI-B2 CRISPR-Cas system. In some implementations, the Type VI CRISPR-Cas system is a VI-C CRISPR-Cas system. In some implementations, the Type VI CRISPR-Cas system is a VI-D CRISPR-Cas system. In some implementations, the Type VI CRISPR-Cas system includes a Cas13a (C2c2), Cas13b (Group 29/30), Cas13c, and/or Cas13d.

### Guide Molecules

The following include general design principles that may be applied to the guide molecule. The terms guide molecule, guide sequence and guide polynucleotide refer to polynucleotides capable of guiding Cas to a target genomic locus and are used interchangeably. In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some implementations, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some implementations, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap. genomics. org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some implementations, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some example implementations, the target sequence may be a sequence within an RNA molecule selected from the group consisting of mRNA, pre-mRNA, and rRNA. In some example implementations, the target sequence may be a sequence within an RNA molecule selected from the group consisting of ncRNA, and lncRNA. In some more example implementations, the target sequence may be a sequence within an mRNA molecule or a pre-mRNA molecule.

In some implementations, a nucleic acid-targeting guide is selected to reduce the degree secondary structure within the nucleic acid-targeting guide. In some implementations, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold. Another example folding algorithm is the online webserver RNAfold, developed at Institute for Theoretical Chemistry at the University of Vienna, using the centroid structure prediction algorithm.

In one example implementation, a guide RNA or crRNA may comprise, consist essentially of, or consist of a direct repeat (DR) sequence and a guide sequence or spacer sequence. In another example implementation, the guide RNA or crRNA may comprise, consist essentially of, or consist of a direct repeat sequence fused or linked to a guide sequence or spacer sequence. In another example implementation, the direct repeat sequence may be located upstream (i.e., 5′) from the guide sequence or spacer sequence. In other implementations, the direct repeat sequence may be located downstream (i.e., 3′) from the guide sequence or spacer sequence.

In one example implementation, the crRNA comprises a stem loop, preferably a single stem loop. In one example implementation, the direct repeat sequence forms a stem loop, preferably a single stem loop.

In one example implementation, the spacer length of the guide RNA is from 15 to 35 nt. In another example implementation, the spacer length of the guide RNA is at least 15 nucleotides. In another example implementation, the spacer length is from 15 to 17 nucleotides (nt), e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some implementations, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5% 99%, or higher. In some implementations, the tracr sequence is about or more than about 5, 6, 7, 8, 9, 10, 11, 12, 13, 14, 15, 16, 17, 18, 19, 20, 25, 30, 40, 50, or more nucleotides in length. In some implementations, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some implementations, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some implementations, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some implementations, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some implementations according to the disclosure, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this disclosure. Various modifications may be used to increase the specificity of binding to the target sequence and/or increase the activity of the Cas protein and/or reduce off-target effects.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some implementations, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences. Instead, many rely on PFSs, which are discussed elsewhere herein. In one example implementation, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the implementations, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system.

In an example implementation, the CRISPR effector protein may recognize a 3′ PAM. In one example implementation, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein. As further detailed herein, the skilled person will understand that Cas13 proteins may be modified analogously. A pool of sgRNAs may be created, tiling across all possible target sites of a panel of six endogenous mouse and three endogenous human genes and quantitatively assessed their ability to produce null alleles of their target gene by antibody staining and flow cytometry. Optimization of the PAM may improve activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Experimental approaches to PAM identification can include, but are not limited to, plasmid depletion assays, screened by a high-throughput in vivo model called PAM-SCNAR, and negative screening.

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference.

Some Type VI proteins, such as subtype B, have 5′-recognition of D (G, T, A) and a 3′-motif requirement of NAN or NNA. One example is the Cas13b protein identified in Bergeyella zoohelcum (BzCas13b).

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some implementations, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example implementation, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:13) or PKKKRKVEAS (SEQ ID NO:14); the NLS from nucleoplasmin (e.g., the nucleoplasmin bipartite NLS with the sequence KRPAATKKAGQAKKKK (SEQ ID NO:15)); the c-myc NLS having the amino acid sequence PAAKRVKLD (SEQ ID NO:16) or RQRRNELKRSP (SEQ ID NO:17); the hRNPA1 M9 NLS having the sequence NQSSNFGPMKGGNFGGRSSGPYGGGGQYFAKPRNQGGY (SEQ ID NO:18); the sequence RMRIZFKNKGKDTAELRRRRVEVSVELRKAKKDEQILKRRN V (SEQ ID NO:19) of the IBB domain from importin-alpha; the sequences VSRKRPRP (SEQ ID NO:20) and PPKKARED (SEQ ID NO:21) of the myoma T protein; the sequence PQPKKKPL (SEQ ID NO:22) of human p53; the sequence SALIKKKKKMAP (SEQ ID NO:23) of mouse c-abl IV; the sequences DRLRR (SEQ ID NO:24) and PKQKKRK (SEQ ID NO:25) of the influenza virus NS1; the sequence RKLKKKIKKL (SEQ ID NO:26) of the Hepatitis virus delta antigen; the sequence REKKKFLKRR (SEQ ID NO:27) of the mouse Mxl protein; the sequence KRKGDEVDGVDEVAKKKSKK (SEQ ID NO:28) of the human poly(ADP-ribose) polymerase; and the sequence RKCLQAGMNLEARKTKK (SEQ ID NO:29) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some implementations, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some implementations, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In example implementations of the Cas proteins, an NLS attached to the C-terminal of the protein.

In some implementations, the CRISPR-Cas protein and a functional domain protein (described further herein) are delivered to the cell or expressed within the cell as separate proteins. In these implementations, each of the CRISPR-Cas and functional domain protein can be provided with one or more NLSs as described herein. In some implementations, the CRISPR-Cas and functional domain protein are delivered to the cell or expressed with the cell as a fusion protein. In these implementations one or both of the CRISPR-Cas and functional domain protein is provided with one or more NLSs. Where the functional domain protein is fused to an adaptor protein (such as MS2) as described above, the one or more NLS can be provided on the adaptor protein, provided that this does not interfere with aptamer binding. In particular implementations, the one or more NLS sequences may also function as linker sequences between the functional domain protein and the CRISPR-Cas protein.

In some implementations, guides of the disclosure comprise specific binding sites (e.g. aptamers) for adapter proteins, which may be linked to or fused to a functional domain protein or catalytic domain thereof. When such a guide forms a CRISPR complex (e.g., CRISPR-Cas protein binding to guide and target), the adapter proteins bind, and the functional domain protein or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some implementations, a component (e.g., the dead Cas protein, the functional domain protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said functional domain protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### CRISPR-Cas Cleavage

In one example implementation, the CRISPR-Cas system may induce a double- or single-stranded break at a designated site in the target sequence. The CRISPR-Cas system may introduce an indel, which, as used herein, refers to insertions or deletions of the DNA at particular locations on the chromosome. The site of CRISPR-Cas cleavage, for most CRISPR-Cas systems, is dictated by distance from a protospacer-adjacent motif (PAM). Accordingly, a guide sequence may be selected to direct the CRISPR-Cas system to induce cleavage at a desired target site at or near the one or more variants.

### NHEJ-Based Editing

In one example implementation, the CRISPR-Cas system is used to introduce one or more insertions or deletions to a target sequence on the gene or enhancer associated with the gene such that one or more indels or insertions reduce expression or activity of the one or more polypeptides. More than one guide sequence may be selected to insert multiple insertion, deletions, or combination thereof. Likewise, more than one Cas protein type may be used, for example, to maximize targets sites adjacent to different PAMs. In one example implementation, a guide sequence is selected that directs the CRISPR-Cas system to make one or more insertions or deletions within the enhancer region. In one example implementation, a guide is selected that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs upstream of an enhancer controlling expression of a target gene. In one example implementation, a guide sequence is selected to that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene. In one example implementation, a guide sequence is selected that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene. In one example implementation, a guide sequence is selected that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene.

### HDR Template Based Editing

In one example implementation, a donor template is provided to replace a genomic sequence in a target gene or sequence controlling expression of the target gene. A donor template may comprise an insertion sequence flanked by two homology regions. The insertion sequence comprises an edited sequence to be inserted in place of the target sequence (e.g., a portion of genomic DNA to be edited). The homology regions comprise sequences that are homologous to the genomic DNA strands at the site of the CRISPR-Cas induced double-strand break. Cellular HDR mechanisms then facilitate insertion of the insertion sequence at the site of the DSB.

Accordingly, in certain example implementations, a donor template and guide sequence are selected to direct excision and replacement of a section of genome DNA comprising an enhancer controlling expression of a target gene or a section of genome DNA within the gene that is required for activity of the target gene. In one example implementation, the insertion sequence comprises a transcription factor binding site that recruits a repressor to the gene.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an implementation, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an implementation, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an implementation, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g. about or more than about 1, 5, 10, 15, 20, 25, 30, 35, 40, 45, 50, 60, 70, 80, 90, 100 or more nucleotides). In some implementations, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example implementation, one or both homology arms may be shortened to avoid including certain sequence repeat elements. For example, a 5′ homology arm may be shortened to avoid a sequence repeat element. In other implementations, a 3′3′ homology arm may be shortened to avoid a sequence repeat element. In some implementations, both the 5′ and the 3′3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques.

In one example implementation, a donor template is a single-stranded oligonucleotide. When using a single-stranded oligonucleotide, 5′ and 3′3′ homology arms may range up to about 2200 base pairs (bp) in length, e.g., at least 25, 50, 75, 100, 125, 150, 175, or 200 bp in length.

### Templates

In some implementations, a composition for engineering cells comprises a template, e.g., a recombination template. A template may be a component of another vector as described herein, contained in a separate vector, or provided as a separate polynucleotide. In some implementations, a recombination template is designed to serve as a template in homologous recombination, such as within or near a target sequence nicked or cleaved by a nucleic acid-targeting effector protein as a part of a nucleic acid-targeting complex.

In an implementation, the template nucleic acid alters the sequence of the target position. In an implementation, the template nucleic acid results in the incorporation of a modified, or non-naturally occurring base into the target nucleic acid.

The template sequence may undergo a breakage mediated or catalyzed recombination with the target sequence. In an implementation, the template nucleic acid may include sequence that corresponds to a site on the target sequence that is cleaved by a Cas protein mediated cleavage event. In an implementation, the template nucleic acid may include a sequence that corresponds to both, a first site on the target sequence that is cleaved in a first Cas protein mediated event, and a second site on the target sequence that is cleaved in a second Cas protein mediated event.

In some implementations, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In some implementations, the template nucleic acid can include a sequence which results in an alteration in a non-coding sequence, e.g., an alteration in an exon or in a 5′ or 3′3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 1 80+/−10, 190+/−10, 200+/−10, 210+/−10, or 220+/−10 nucleotides in length. In an implementation, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an implementation, the template nucleic acid is 10 to 1000, 20 to 900, 30 to 800, 40 to 700, 50 to 600, 50 to 500, 50 to 400, 50 to 300, 50 to 200, or 50 to 100 nucleotides in length.

In some implementations, the template polynucleotide is complementary to a portion of a polynucleotide comprising the target sequence. When optimally aligned, a template polynucleotide might overlap with one or more nucleotides of a target sequences (e.g., about, or more than about, 1, 5, 10, 15, 20, 25, 30, 35, 40, 45, 50, 60, 70, 80, 90, 100 or more nucleotides). In some implementations, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In some implementations, one or both homology arms may be shortened to avoid including certain sequence repeat elements. For example, a 5′ homology arm may be shortened to avoid a sequence repeat element. In other implementations, a 3′3′ homology arm may be shortened to avoid a sequence repeat element. In some implementations, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques.

In some implementations, a template nucleic acid for correcting a mutation may designed for use as a single-stranded oligonucleotide. When using a single-stranded oligonucleotide, 5′ and 3′ homology arms may range up to about 2200 base pairs (bp) in length, e.g., at least 25, 50, 75, 100, 125, 150, 175, or 200 bp in length.

### Specialized Cas-Based Systems

In some implementations, the system is a Cas-based system that is capable of performing a specialized function or activity. For example, the Cas protein may be fused, operably coupled to, or otherwise associated with one or more functionals domains. In certain example implementations, the Cas protein may be a catalytically dead Cas protein (“dCas”) and/or have nickase activity. A nickase is a Cas protein that cuts only one strand of a double stranded target. In such implementations, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4× domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9, Cas12, and Cas13 are known in the art.

In some implementations, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some implementations, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In implementations having two or more functional domains, each of the two can be positioned at or near or in proximity to a terminus of the effector protein (e.g., a Cas protein). In some implementations, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some implementations, all the functional domains are the same. In some implementations, all of the functional domains are different from each other. In some implementations, at least two of the functional domains are different from each other. In some implementations, at least two of the functional domains are the same as each other.

### Split CRISPR-Cas Systems

In one example implementation, the CRISPR-Cas system is a split CRISPR-Cas system. Split CRISPR-Cas proteins are set forth herein and in documents incorporated herein by reference in further detail herein. In some implementations, each part of a split CRISPR protein is attached to a member of a specific binding pair, and when bound with each other, the members of the specific binding pair maintain the parts of the CRISPR protein in proximity. In some implementations, each part of a split CRISPR protein is associated with an inducible binding pair. An inducible binding pair is one which is capable of being switched “on” or “off” by a protein or small molecule that binds to both members of the inducible binding pair. In some implementations, CRISPR proteins may preferably split between domains, leaving domains intact. In particular implementations, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In one example implementation, the gene editing system configured to modify the one or more target genes disclosed herein is a base editing system. In one example implementation, a Cas protein is connected or fused to a nucleotide deaminase. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems. Accordingly, in one example implementation, the base editing system edits the target gene to reduce or eliminate its expression.

In one example implementation, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair and ABEs convert an A·T base pair to a G·C base pair. Collectively, CBEs and ABEs can mediate all four possible transition mutations (C to T, A to G, T to C, and G to A). In some implementations, the base editing system includes a CBE and/or an ABE. In some implementations, a polynucleotide of the present disclosure described elsewhere herein can be modified using a base editing system. Base editors also generally do not need a DNA donor template and/or rely on homology-directed repair. Upon binding to a target locus in the DNA, base pairing between the guide RNA of the system and the target DNA strand leads to displacement of a small segment of ssDNA in an “R-loop.” DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template.

In one example implementation, the base editing system may be an RNA base editing system. As with DNA base editors, a nucleotide deaminase capable of converting nucleotide bases may be fused to a Cas protein. However, in these implementations, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example implementations, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response.

### Prime Editors

In one example implementation, the gene editing system configured to modify the target genes is a prime editing system. Prime editing advantageously provides lower off-target editing than a Cas9 nuclease system. In example implementations, the target gene is edited to introduce a stop codon, mutate an essential residue (e.g., an active site residue in a target enzyme, a residue essential for protein-protein binding, or a residue required for modification), or introduce a frameshift that inactivates the gene. In example a regulatory sequence, such as an enhancer sequence is edited to reduce or eliminate binding of a transcription factor.

In one example implementation, a genomic sequence in a target gene or sequence controlling expression of the target gene is replaced or deleted using a prime editing system. Like base editing systems, prime editing systems can be capable of targeted modification of a polynucleotide without generating double stranded breaks. Further prime editing systems are capable of all 12 possible combination swaps. Prime editing may operate via a “search-and-replace” methodology and can mediate targeted insertions, deletions, of all 12 possible base-to-base conversion and combinations thereof. Generally, a prime editing system, as exemplified by PE1, PE2, and PE3, can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Implementations that can be used with the present disclosure include these and variants thereof. Prime editing can have the advantage of lower off-target activity.

In some implementations, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide.

In some implementations, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some implementations, the Cas polypeptide is a Class 2, Type V Cas polypeptide. In some implementations, the Cas polypeptide is a Cas9 polypeptide (e.g., is a Cas9 nickase). In some implementations, the Cas polypeptide is fused to the reverse transcriptase. In some implementations, the Cas polypeptide is linked to the reverse transcriptase.

In some implementations, the prime editing system can be a PE1 system or variant thereof, a PE2 system or variant thereof, or a PE3 (e.g., PE3, PE3b) system.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length.

Prime editing can also include a system that uses a prime editor (PE) protein and two prime editing guide RNAs (pegRNAs), such that, the two pegRNAs template the synthesis of complementary DNA flaps on opposing strands of genomic DNA, which replace the endogenous DNA sequence between the PE-induced nick sites. Thus, use of two pegRNAs allows for larger insertions because of the two overlapping 3′ flaps created by the two nicked sites. The system can be combined with a site-specific serine recombinase to allow targeted integration of gene-sized DNA plasmids (greater than 5,000 bp) and targeted sequence inversions of 40 kb in human cells. In one example implementation, the system can be used to insert or replace a sequence into one or more target genes. In example implementations, the insertion or replacement results in an inactive target gene or less active form of the target gene. In one example implementation, the system is used to replace all or a portion of the entire target gene. In one example implementation, the system is used to replace all or a portion of an enhancer controlling the target gene expression.

### CRISPR-Directed Integrase

In example implementations, the prime editing system inserts a serine integrase attachment site for large, multiplexed gene insertion without reliance on DNA repair pathways. This system is a variation of prime editing that includes all of the components of prime editing, but with an integrase. Serine integrases typically insert sequences containing an attP attachment site into a target containing the related attB attachment site. By using programmable genome editing to place integrase landing sites at desired locations in the genome, this system directly guides the activity of the associated integrase to the specific genomic site. In one implementation, pegRNAs including attB sequences are used to insert the sites at desired locations in the genome. In one implementation, the system uses a Cas enzyme-reverse transcriptase-integrase fusion protein to directly recruit the integrase to the target site.

“Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP′ and POB′, respectively. In some representations herein, the “O” is omitted and attB and attP, for example, are designated as BB′ and PP′, respectively.

In example implementations, the recombinase of the present disclosure is a serine integrase. In example implementations, serine integrases specifically recombine when recognizing the two attachment sites specific for the integrase. In example implementations, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors integrase is unidirectional. In addition, DNA substrates identified by serine integrases (attP and attB) are relatively short (30-50 bp) and have a minimal length of approximately 34-40 base pairs (bp). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present disclosure include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable to the present disclosure include C31 integrase, Bxbl, φBT1 integrase, A118, TP901-1, and R4 and the corresponding recombination sites for each. In some implementations, a functional domain of the serine integrase is used.

In one example implementation, the system can be used to insert or replace a sequence into one or more target genes. In example implementations, the insertion or replacement results in an inactive target gene or less active form of the target gene. In one example implementation, the system is used to replace all or a portion of the entire target gene. In one example implementation, the system is used to replace all or a portion of an enhancer controlling the target gene expression.

### CRISPR Associated Transposase (CAST) Systems

In one example implementation, the gene editing system configured to modify the one or more target genes is a CRISPR associated transposase system (CAST). In one example implementation, the CAST system can be used to insert or replace a sequence into one or more target genes. In example implementations, the insertion or replacement results in an inactive target gene or less active form of the target gene. In one example implementation, a CAST system is used to replace all or a portion of an enhancer controlling the target gene expression. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems.

### OMEGA Systems

In one example implementation, the gene editing system configured to modify the one or more target genes is a transposon-encoded RNA-guided nuclease system, referred to herein as OMEGA (obligate mobile element-guided activity). OMEGA systems include, but are not limited to IscB, IsrB, TnpB systems.

In some implementations, the nucleic acid-guided nucleases herein may be an IscB protein. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins.

In some implementations, the nucleic acid-guided nucleases herein may be an IsrB (Insertion sequence RuvC-like OrfB) protein. IsrB refers to a group of shorter, ˜350 aa IscB homologs that are also encoded in IS200/2305 superfamily transposons. These proteins contain a PLMP domain and split RuvC but lack the HNH domain.

In some implementations, the nucleic acid-guided nucleases herein may be a TnpB protein. TnpB is a putative endonuclease distantly related to iscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or W systems or complexes for short. TnpB systems are a distinct type of W system, which further include IscB, IsrB, and IshB systems. The nucleic acid component of W sytems is structurally distinct from other RNA-guided nucleases, such as CRISPR-Cas systems, and may also be referred to as a wRNA. In certain example implementations, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

### Epigenetic Editing

In one example implementation, the one or more agents is an epigenetic modification polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides disclosed herein. As used herein, “linked to or otherwise capable of associating with” refers to a fusion protein or a recruitment domain or the adaptor protein, such as an aptamer (e.g., MS2) or an epitope tag. The recruitment domain or the adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example implementations, the DNA binding domain is a programmable DNA binding protein linked to or otherwise capable of associating with an epigenetic modification domain. Programmable DNA binding proteins for modifying the epigenome include, but are not limited to CRISPR systems, transcription activator-like effectors (TALEs), Zn finger proteins and meganucleases. In example implementations, the DNA binding domain is a nuclease-deficient RNA-guided DNA endonuclease enzyme or a nuclease-deficient endonuclease enzyme. In example implementations, a CRISPR system having an inactivated nuclease activity (e.g., dCas) is used as the DNA binding domain.

In example implementations, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain. Example epigenetic modification domains can be obtained from, but are not limited to chromatin modifying enzymes, such as, DNA methyltransferases (e.g., DNMT1, DNMT3a and DNMT3b), TET1, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SET8), MMSET, EZH2, and MLL family members), DOT1L, LSD1, Jumonji demethylases (e.g., KDM5A (JARID1A), KDM5C (JARID1C), and KDM6A (UTX)), kinases (e.g., Haspin, VRK1, PKCa, PKCP, PIM1, IKKa, Rsk2, PKB/Akt, Aurora B, MSK1/2, JNK1, MLTKa, PRK1, Chkl, Dlk/ZIP, PKC6, MST1, AMPK, JAK2, Abl, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1).

In example implementations, histone acetylation is targeted to a target sequence using a CRISPR system (see, e.g., Hilton I B, et al. Epigenome editing by a CRISPR-Cas9-based acetyltransferase activates genes from promoters and enhancers. In example implementations, histone deacetylation is targeted to a target sequence. In example implementations, histone methylation is targeted to a target sequence. In example implementations, histone demethylation is targeted to a target sequence. In example implementations, histone phosphorylation is targeted to a target sequence. In example implementations, DNA methylation is targeted to a target sequence. In example implementations, DNA demethylation is targeted to a target sequence using a CRISPR system. In example implementations, DNA demethylation is targeted to a target sequence.

Example epigenetic modification domains can be obtained from, but are not limited to transcription activators, such as, VP64, p65, HSF1, and RTA. Example epigenetic modification domains can be obtained from, but are not limited to transcription repressors, such as, e.g., KRAB.

In example implementations, the epigenetic modification domain linked to a DNA binding domain recruits an epigenetic modification protein to a target sequence. In example implementations, a transcriptional activator recruits an epigenetic modification protein to a target sequence. For example, VP64 can recruit DNA demethylation, increased H3K27ac and H3K4me. In example implementations, a transcriptional repressor protein recruits an epigenetic modification protein to a target sequence. For example, KRAB can recruit increased H3K9me3. In an example implementation, methyl-binding proteins linked to a DNA binding domain, such as MBD1, MBD2, MBD3, and MeCP2 recruits an epigenetic modification protein to a target sequence. In an example implementation, Mi2/NuRD, Sin3A, or Co-REST recruit HDACs to a target sequence.

In example implementations, the epigenetic modification domain can be a eukaryotic or prokaryotic (e.g., bacteria or Archaea) protein. In example implementations, the eukaryotic protein can be a mammalian, insect, plant, or yeast protein and is not limited to human proteins (e.g., a yeast, insect, plant chromatin modifying protein, such as yeast HATs, HDACs, methyltransferases, etc.).

In one aspect of the disclosure, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In implementations, the epigenetic modification polypeptide comprises the nuclease-deficient RNA-guided DNA endonuclease enzyme. In implementations, the fusion protein comprises the nuclease-deficient DNA endonuclease enzyme.

In some implementations, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9.

In some implementations, the present disclosure provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In implementations, the fusion protein comprises the nuclease-deficient RNA-guided DNA endonuclease enzyme. In implementations, the fusion protein comprises the nuclease-deficient DNA endonuclease enzyme.

In some implementations, the present disclosure provides a method of activating a target nucleic acid sequence in a cell, the method comprising: (i) delivering a first polynucleotide encoding a epigenetic modification polypeptide described herein including implementations thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Zinc Finger Nucleases

In some implementations, the target gene is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. Increased cleavage specificity can be attained with decreased off target activity by use of paired ZFN heterodimers, each targeting different nucleotide sequences separated by a short spacer. ZFPs can also be designed as transcription activators and repressors and have been used to target many genes in a wide variety of organisms.

### TALE Nucleases

In some implementations, a TALE nuclease or TALE nuclease system can be used to modify a target gene. In some implementations, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous implementations the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous implementation, z is at least 5 to 40. In a further advantageous implementation, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some implementations, monomers with an RVD of IG can preferentially bind to T. Thus, the number and order of the polypeptide monomer repeats in the nucleic acid binding domain of a TALE determines its nucleic acid target specificity. In some implementations, monomers with an RVD of NS can recognize all four base pairs and can bind to A, T, G or C.

The polypeptides used in methods of the disclosure can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some implementations, polypeptide monomers having RVDs RN, NN, NK, SN, NH, KN, HN, NQ, HH, RG, KH, RH and SS can preferentially bind to guanine. In some implementations, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some implementations, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some implementations, the RVDs that have high binding specificity for guanine are RN, NH RH and KH. Furthermore, polypeptide monomers having an RVD of NV can preferentially bind to adenine and guanine. In some implementations, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the disclosure will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in some implementations, the TALE polypeptides described herein further comprise an N-terminal capping region and/or a C-terminal capping region.

An exemplary amino acid sequence of a N-
terminal capping region is:
(SEQ ID NO: 30)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N
An exemplary amino acid sequence of a C-terminal
capping region is:
(SEQ ID NO: 31)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in some implementations, fragments of the N-terminal and/or C-terminal capping regions are included in the TALE polypeptides described herein.

In some implementations, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some implementations, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. C-terminal capping region fragments that include the C-terminal 68 amino acids enhance binding activity equal to the full-length capping region, while fragments that include the C-terminal 20 amino acids retain greater than 50% of the efficacy of the full-length capping region.

In some implementations, the capping regions of the TALE polypeptides described herein do not need to have identical sequences to the capping region sequences provided herein. Thus, in some implementations, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some example implementations, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some implementations described herein, the TALE polypeptides of the disclosure include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the disclosure may be used to target the one or more functions or activities mediated by the effector domain to a particular target DNA sequence to which the nucleic acid binding domain specifically binds.

In some implementations of the TALE polypeptides described herein, the activity mediated by the effector domain is a biological activity. For example, in some implementations the effector domain is a transcriptional inhibitor (i.e., a repressor domain), such as an mSin interaction domain (SID). SID4X domain or a Krüppel-associated box (KRAB) or fragments of the KRAB domain. In some implementations, the effector domain is an enhancer of transcription (i.e., an activation domain), such as the VP16, VP64 or p65 activation domain. In some implementations, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some implementations, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other example implementations of the disclosure may include any combination of the activities described herein.

### Meganucleases

In some implementations, a meganuclease or system thereof can be used to modify a target gene. Meganucleases, which are endodeoxyribonucleases characterized by a large recognition site (double-stranded DNA sequences of 12 to 40 base pairs).

### ARCUS Based Editing

In one example implementation, a target gene is modified with an ARCUS base editing system.

### RNAi and Antisense Oligonucleotides (ASO)

In some implementations, one or more overexpressed genes in striatal projection neurons (SPNs) that have CAG somatic expansion greater than 180 CAGs are targeted with RNAi or antisense oligonucleotides (ASO). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one example implementation, the mRNA levels are decreased by at least about 70%, about 80%, about 90%, about 95%, about 99%, about 100%. Additionally, inhibitory nucleic acid molecules such as RNAi and ASOs can be used in vivo.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one implementation, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one implementation, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA, comprises a dsRNA molecule.

Antisense therapy is a form of treatment that uses antisense oligonucleotides (ASOs) to target messenger RNA (mRNA). ASOs are capable of altering mRNA expression through a variety of mechanisms, including ribonuclease H mediated decay of the pre-mRNA, direct steric blockage, and exon content modulation through splicing site binding on pre-mRNA. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Commonly used antisense mechanisms to degrade target RNAs include RNase H1-dependent and RISC-dependent mechanisms. Example ASOs include Locked Nucleic Acid (LNA), Peptide Nucleic Acid (PNA), and morpholinos.

### Small Molecules

In example implementations, one or more overexpressed genes in striatal projection neurons (SPNs) that have CAG somatic expansion greater than 180 CAGs are targeted using a small molecule. In example implementations, receptors are targeted with small molecules that block ligand binding. In example implementations, a target protein is targeted with a degrader molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Example small organic molecules range in size up to about 5000 Da, e.g., up to about 4000, up to about 3000 Da, up to about 2000 Da, up to about 1000 Da, or less (e.g., up to about 900, 800, 2400, 2300 or up to about 2100 Da). In some implementations, the small molecule may act as an antagonist or agonist (e.g., blocking an enzyme active site or activating a receptor by binding to a ligand binding site).

### Small Molecule Degraders

One type of small molecule applicable to the present disclosure is a degrader molecule. The terms “degrader” and “degrader molecule” refer to all compounds capable of specifically targeting a protein for degradation (e.g., ATTEC, AUTAC, LYTAC, or PROTAC). Examples include proteolysis pargeting chimera (PROTAC) technology, which is a rapidly emerging alternative therapeutic strategy with the potential to address many of the challenges currently faced in modern drug development programs. PROTAC technology employs small molecules that recruit target proteins for ubiquitination and removal by the proteasome. In some implementations, LYTACs are particularly advantageous for cell surface proteins. PROTACs can be synthesized for any target of interest, as evidenced by the hundreds of PROTACS available. PROTACs have been demonstrated to be safe, efficacious, and to have clinical efficacy with meaningful benefits for patients. PROTACs can be designed using fully synthetic, rationally designed small molecules. In example implementations, any druggable gene described herein can be targeted by rationale design starting with the drugs that bind to the gene products. In other example implementations, the targeting molecule does not need to inhibit the gene product and small molecule libraries can easily be screened for molecules that bind to the target.

### PHICS

In example implementations, one or more overexpressed genes in striatal projection neurons (SPNs) that have CAG somatic expansion greater than 180 CAGs are targeted with chimeric molecules that recruit enzymes to the target protein by a similar mechanism as PROTACs. In implementations, the enzyme is a kinase, a phosphatase, transferase, glycosyltransferase, ligase, a histone acetylase (HAT) or histone deacetylase (HDAC), a hydroxylase, a glutamine synthetase adenyl transferase (GSATase), an enzyme catalyzing hydroxylation of protein residues, an oxygenase, or a sulfotransferase. Phosphorylation-inducing chimeric small molecules (PHICS) can enable a kinase to act at a new cellular location or phosphorylate non-native substrates (neo-substrates) and/or sites (neo-phosphorylations). PHICS are formed by linking small-molecule binders of the kinase or the phosphatase and the target protein. The molecule that binds the target protein is the same as for PROTACs described herein and can be rationally designed in the same way. In example implementations, modulating modifications at sites that regulate the target protein or at neo-sites inactivates or reduces the function of the target protein.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A method comprising:
generating labeled amplicons of a variable repeat region of a gene, said generating using primers that introduce at least one molecular label to respective nucleic acid molecules of origin of a biological sample, the at least one molecular label including a unique molecular identifier having a nucleotide sequence that varies between the respective nucleic acid molecules of origin;
sequencing the labeled amplicons to generate sequencing reads having the at least one molecular label incorporated; and
generating a sequence repeat length distribution of the variable repeat region in at least a portion of the biological sample based on the sequencing reads.

2. The method of claim 1, wherein generating the sequence repeat length distribution of the variable repeat region in at least the portion of the biological sample based on the sequencing reads comprises:
identifying read families based on the at least one molecular label, each read family comprising a subset of the sequencing reads having a matched sequence for the at least one molecular label;
generating molecule-specific consensus sequences based on the identified read families, each molecule-specific consensus sequence corresponding to a sequence of a single nucleic acid molecule of origin of the biological sample;
determining consensus repeat lengths for respective molecule-specific consensus sequences; and
generating the sequence repeat length distribution based on the consensus repeat lengths.

3. The method of claim 1, wherein the labeled amplicons comprise cDNA of the variable repeat region, and wherein the respective nucleic acid molecules of origin are RNA transcripts of the biological sample.

4. The method of claim 1, wherein the at least one molecular label further comprises a cell barcode that varies based on a cell of origin of the labeled amplicons in the biological sample.

5. The method of claim 1, wherein the at least one molecular label further comprises at least one index sequence that is specific to the biological sample.

6. The method of claim 1, wherein the primers that introduce the at least one molecular label to the respective nucleic acid molecules of origin are used during a reverse transcription reaction, and the method further comprises amplifying the labeled amplicons during a transcriptome amplification reaction.

7. The method of claim 6, wherein the transcriptome amplification reaction uses spike-in primers targeting the variable repeat region of the gene.

8. The method of claim 6, wherein the method further comprises enriching the amplified labeled amplicons for the variable repeat region of the gene during a targeted amplification reaction.

9. The method of claim 8, wherein the targeted amplification reaction uses gene-specific primers for the variable repeat region of the gene, and at least one of a forward primer of the gene-specific primers or a reverse primer of the gene-specific primers includes an affinity purification tag at a 5′ end.

10. The method of claim 1, wherein the respective nucleic acid molecules are molecules of genomic DNA, and the primers that introduce the at least one molecular label to the respective nucleic acid molecules of origin are used during a first amplification reaction having a first number of reaction cycles, and the method further comprises:
amplifying the labeled amplicons during a second amplification reaction having a second number of reaction cycles that is greater than the first number of reaction cycles.

11. The method of claim 1, wherein the gene is associated with a repeat expansion disorder, and wherein the portion of the biological sample is defined by a type of cell.

12. A method comprising:
generating labeled amplicons of a variable length sequence repeat region of a gene, the labeled amplicons having molecular labels uniquely identifying an individual nucleic acid molecule of origin from which a given labeled amplicon is derived;
sequencing the labeled amplicons to generate sequencing reads of the variable length sequence repeat region, the sequencing reads having the molecular labels incorporated;
identifying read families of the sequencing reads based on the molecular labels, each read family comprising a subset of the sequencing reads corresponding to respective individual nucleic acid molecules of origin; and
determining a repeat length of the variable length sequence repeat region for the respective individual nucleic acid molecules of origin based on the read families.

13. The method of claim 12, wherein the respective individual nucleic acid molecules of origin are RNA transcripts, and wherein generating the labeled amplicons of the variable length sequence repeat region of the gene comprises introducing the molecular labels via primers targeting the RNA transcripts in a reverse transcription reaction.

14. The method of claim 12, wherein the respective individual nucleic acid molecules of origin are molecules of genomic DNA, and wherein generating the labeled amplicons of the variable length sequence repeat region of the gene comprises:
introducing the molecular labels via primers targeting the variable length sequence repeat region of the gene during a first amplification reaction having a first number of reaction cycles; and
amplifying the labeled amplicons during a second amplification reaction having a second number of reaction cycles that is greater than the first number of reaction cycles.

15. The method of claim 12, wherein the molecular labels include a cell-specific barcode that uniquely identifies a cell of origin of the labeled amplicons.

16. The method of claim 12, wherein the molecular labels include an index sequence that uniquely identify a biological sample of origin of the labeled amplicons.

17. A method comprising:
generating, via a reverse transcription reaction, labeled amplicons of a targeted variable length sequence repeat region of RNA transcripts from a biological sample, the reverse transcription reaction introducing molecular labels that uniquely label the labeled amplicons derived from individual RNA transcripts of individual nuclei of the biological sample;
preparing, via at least one amplification reaction and at least one purification process, the labeled amplicons for sequencing; and
determining, via the sequencing of the labeled amplicons, sequence repeat lengths of the targeted variable length sequence repeat region of the individual RNA transcripts.

18. The method of claim 17, further comprising generating, based on the determined sequence repeat lengths, a sequence repeat length distribution of the targeted variable length sequence repeat region of the individual RNA transcripts in a per-cell basis.

19. The method of claim 17, wherein the molecular labels include a unique molecular label sequence that distinguishes the labeled amplicons derived from the individual RNA transcripts of a single nucleus from each other and a cell barcode sequence that distinguishes the labeled amplicons derived from different nuclei.

20. The method of claim 19, wherein, determining, via the sequencing of the labeled amplicons, the sequence repeat lengths of the targeted variable length sequence repeat region of the individual RNA transcripts from the biological sample comprises:
generating sequencing data via the sequencing, the sequencing data comprising sequencing reads of the labeled amplicons;
identifying read families based on the cell barcode sequence and the unique molecular label sequence in the sequencing reads, each read family comprising a matched sequence for the cell barcode sequence and the unique molecular label sequence; and
determining the sequence repeat lengths of the targeted variable length sequence repeat region of the individual RNA transcripts based on respective sequence repeat length distributions of the read families.


SEQUENCE LISTING (31 sequences)

SEQ ID NO: 1 - DNA, 21 bp, synthetic construct
  source 1..21: mol_type = other DNA
cccagagccccattcattgcc

SEQ ID NO: 2 - DNA, 22 bp, synthetic construct
  source 1..22: mol_type = other DNA
ggcgaccctggaaaagctgatg

SEQ ID NO: 3 - DNA, 113 bp, synthetic construct
  source 1..113: mol_type = other DNA
  misc_feature 1: note = The nucleotide is connected to a biotin molecule
gtgactggagttcagacgtgtgctcttccgatctccttcgagtccctcaagtccttcgtctcgtgggctcggagatgtgataagagacagccttcgagtccctcaagtccttc

SEQ ID NO: 4 - DNA, 33 bp, synthetic construct
  source 1..33: mol_type = other DNA
acactctttccctacacgacgctcttccgatct

SEQ ID NO: 5 - DNA, 64 bp, synthetic construct
  source 1..64: mol_type = other DNA
tcgtcggcagcgtcagatgtgtataagagacagnnnnnnnnnnnnggctgaggaagctgaggag

SEQ ID NO: 6 - DNA, 68 bp, synthetic construct
  source 1..68: mol_type = other DNA
gtctcgtgggctcggagatgtgtataagagacgnnnnnnnnnnnnccttcgagtccctcaagtccttc

SEQ ID NO: 7 - DNA, 51 bp, synthetic construct
  source 1..51: mol_type = other DNA
  variation 30..37: note = Sequence ranges from eight to twelve nucleotides in length
aatgatacggcgaccaccgagatctacacnnnnnnnntcgtcggcagcgtc

SEQ ID NO: 8 - DNA, 47 bp, synthetic construct
  source 1..47: mol_type = other DNA
  variation 25..32: note = sequence ranges from eight to twelve nucleotides in length
caagcagaagacggcatacgagatnnnnnnnngtctcgtgggctcgg

SEQ ID NO: 9 - DNA, 12 bp, synthetic construct
  source 1..12: mol_type = other DNA
gactccccagca

SEQ ID NO: 10 - DNA, 12 bp, synthetic construct
  source 1..12: mol_type = other DNA
atagttggcgac

SEQ ID NO: 11 - DNA, 12 bp, synthetic construct
  source 1..12: mol_type = other DNA
ctgtaagtgcgg

SEQ ID NO: 12 - DNA, 12 bp, synthetic construct
  source 1..12: mol_type = other DNA
gtacccagacag

SEQ ID NO: 13 - protein, 7 aa, simian vacuolating virus 40
PKKKRKV

SEQ ID NO: 14 - protein, 10 aa, simian vacuolating virus 40
PKKKRKVEAS

SEQ ID NO: 15 - protein, 16 aa, unidentified
KRPAATKKAGQAKKKK

SEQ ID NO: 16 - protein, 9 aa, unidentified
PAAKRVKLD

SEQ ID NO: 17 - protein, 11 aa, unidentified
RQRRNELKRSP

SEQ ID NO: 18 - protein, 38 aa, Homo sapiens
NQSSNFGPMKGGNFGGRSSGPYGGGGQYFAKPRNQGGY

SEQ ID NO: 19 - protein, 42 aa, unidentified
RMRIZFKNKGKDTAELRRRRVEVSVELRKAKKDEQILKRRNV

SEQ ID NO: 20 - protein, 8 aa, unidentified
VSRKRPRP

SEQ ID NO: 21 - protein, 8 aa, unidentified
PPKKARED

SEQ ID NO: 22 - protein, 8 aa, Homo sapiens
PQPKKKPL

SEQ ID NO: 23 - protein, 12 aa, Mus musculus
SALIKKKKKMAP

SEQ ID NO: 24 - protein, 5 aa, influenza virus
  source 1..5: note = NS1
DRLRR

SEQ ID NO: 25 - protein, 7 aa, influenza virus
  source 1..7: note = NS1
PKQKKRK

SEQ ID NO: 26 - protein, 10 aa, hepatitis virus
  source 1..10: note = delta antigen
RKLKKKIKKL

SEQ ID NO: 27 - protein, 10 aa, Mus musculus
REKKKFLKRR

SEQ ID NO: 28 - protein, 20 aa, Homo sapiens
KRKGDEVDGVDEVAKKKSKK

SEQ ID NO: 29 - protein, 17 aa, Homo sapiens
RKCLQAGMNLEARKTKK

SEQ ID NO: 30 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 31 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS